SEQUENCE LISTING (12 sequences)

SEQ ID NO: 1 - protein, 5 aa, synthetic construct
TYWLG

SEQ ID NO: 2 - protein, 17 aa, synthetic construct
IMSPVDSDIRYSPSFQG

SEQ ID NO: 3 - protein, 10 aa, synthetic construct
RRPGQGYFDF

SEQ ID NO: 4 - protein, 11 aa, synthetic construct
RASQGISSWLA

SEQ ID NO: 5 - protein, 7 aa, synthetic construct
AASSLQS

SEQ ID NO: 6 - protein, 9 aa, synthetic construct
QQYNIYPYT

SEQ ID NO: 7 - protein, 119 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 8 - protein, 108 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 9 - protein, 330 aa, synthetic construct
SSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0228864 A1
(43) Pub. Date: Jul. 17, 2025

(54) THERAPIES FOR THE TREATMENT OF INFLAMMATORY BOWEL DISEASE

(71) Applicant: Gilead Sciences, Inc., Foster City, CA (US)

(72) Inventors: Astrid S. Clarke, San Marcos, CA (US); Dahtsing Kuo, San Francisco, CA (US); David A. Lopez, Sherman Oaks, CA (US); Silke Roedder, Menlo Park, CA (US); Tangsheng Yi, Belmont, CA (US); Jin Zhou, Newton, CT (US); Mark C. Genovese, Sunnyvale, CA (US)

(21) Appl. No.: 18/960,068

(22) Filed: Nov. 26, 2024

Related U.S. Application Data
(60) Provisional application No. 63/604,090, filed on Nov. 29, 2023.

(51) Int. Cl. A61K 31/5377; A61K 39/00; A61K 39/395; A61P 1/04

(52) U.S. Cl. CPC A61K 31/5377; A61K 39/3955; A61P 1/04; A61K 2039/505

(57) ABSTRACT

The present disclosure relates to methods of preventing and/or treating Ulcerative colitis (UC) comprising administering an α4β7 small molecule inhibitor alone and in combination with an anti-IL-12/IL-23 inhibitor.

### CROSS-REFERENCE TO RELATED APPLICATIONS

This application claims the benefit under 35 U.S.C. § 119 (e) of U.S. Provisional Application No. 63/604,090, filed on Nov. 29, 2023, which is hereby incorporated herein by reference in its entirety for all purposes.

### SEQUENCE LISTING

The instant application contains a Sequence Listing which has been submitted electronically in .XML format and is hereby incorporated by reference in its entirety. Said copy, created on Dec. 4, 2024, is named 1463-US—NP_SL.xml and is 12,706 bytes in size.

### FIELD

The present disclosure generally relates to methods for preventing and/or treating gastrointestinal diseases, in particular, inflammatory bowel disease, Crohn's disease, and ulcerative colitis.

### BACKGROUND

Inflammatory bowel disease (IBD) is a chronic, relapsing and remitting inflammatory disorder that substantially impairs the quality of life in patients. IBD includes Crohn's disease (CD) and ulcerative colitis (UC). The pathophysiology of IBD is often described as though it was a single entity; however, important differences exist between UC and CD phenotypes. For example, UC is classified by the extent of colonic involvement, moving from isolated proctitis through proctosigmoiditis to left-sided colitis and further spreading to extensive or pan-colitis. In contrast, CD may affect the entire GI tract from mouth to anus, although the most common segments affected are the terminal ileum and colon.

Treatment of IBD, including UC and/or CD, is dependent on the severity and the location of the disease. Mildly to moderately active distal colitis may be treated with oral aminosalicylates, topical mesalamine, or topical steroids. For moderately active disease, oral corticosteroids, and immunomodulators such as azathioprine and 6-mercaptopurind (6-MP) have been utilized (Danese S, Fiocchi C. Ulcerative colitis. N Engl J Med 2011; 365 (18): 1713-25). For more moderately to severely active disease, patients have been treated with a more advanced therapy, e.g., a tumor necrosis factor-alpha (TNFα) antagonist such as infliximab or an α4β7 integrin antagonist such as vedolizumab.

Biologic treatments for IBD have also become available including, for example, adalimumab (an antibody against TNF«), golimumab (an antibody against TNF&), and ustekinumab (an antibody against interleukin (IL)-12 and/or IL-23). These therapies have various mechanisms of action.

While on the one hand, the introduction of these biologic therapies has significantly improved response rates in patients, many patients receiving these therapies develop neutralizing antibodies to the biologic agents with resulting loss of efficacy over time. In addition, traditional therapeutics for IBD have also been associated with safety issues, including but not limited to anaphylaxis, malignance, increased risks of infection, progressive multifocal leukoencephalopathy, and liver injury. Accordingly, there remains a significant unmet need for safe and effective treatments for IBD, including CD and UC.

### SUMMARY

Disclosed herein is a method of treating and/or preventing inflammatory bowel disease (IBD) in a patient in need thereof, comprising administering to the patient a therapeutically effective amount of an alpha-4 beta-7 (α4β7) integrin small molecule inhibitor, or a pharmaceutically acceptable salt thereof. In some embodiments, provided herein, the inflammatory bowel disease can be any inflammatory bowel disease, but not limited to, UC and CD. Also disclosed herein is a method of treating and/or preventing IBD in a patient in need thereof, comprising administering to the patient a therapeutically effective amount of α4β7 integrin small molecule inhibitor, or a pharmaceutically acceptable salt thereof, and a therapeutically effective amount of an anti-interleukin-12/interleukin-23 (IL-12/IL-23) antibody. In some embodiments, provided herein, the inflammatory bowel disease can be any inflammatory bowel disease, but not limited to, UC and CD.

In certain embodiments, provided herein is a method for treating and/or preventing IBD in a subject, comprising administering to the subject a therapeutically effective amount of an α4β7 integrin small molecule inhibitor in combination with a therapeutically effective amount of an anti-IL-12/IL-23 antibody.

In some embodiments provided herein, the method comprises administering the α4β7 integrin small molecule inhibitor at a dose of about 25-200 mg once daily.

In certain embodiments, the method comprises administering an induction dose from 260-520 mg of the anti-IL-12/IL-23 antibody on Day 1. In some embodiments provided herein, the method further comprising, administering a dose of 90 mg of the anti-IL-12/IL-23 antibody at week 8, at week 16, at week 24, at week 32 at week 40 and at week 48. In some embodiments, the anti-IL-12/IL-23 antibody is ustekinumab or a biosimilar thereof.

### BRIEF DESCRIPTION OF THE DRAWINGS

FIG. 1 shows a graph illustrating the mean plasma concentration-time profiles (0-24 hours) by treatment following once-daily dosing of a compound disclosed herein under nonfasting and fasting conditions for 1 day.

FIG. 2 shows a graph illustrating the mean plasma concentration-time profiles (0-96 hours) by treatment following once-daily dosing of a compound disclosed herein under nonfasting and fasting conditions for 14 days.

FIG. 3 shows a graph illustrating the α4β7% RO at Day 1 for the Single Ascending Dose (SAD) Cohorts 1-5 under fasting conditions.

FIG. 4 shows a graph illustrating the α4β7% RO at Day 1 and at Day 14 for the Multiple Ascending Dose (MAD) Cohorts 6-10 under non-fasting (Cohorts 6-9) and fasting conditions (Cohort 10).

FIG. 5 shows a graph illustrating the simulated steady state α4β7% RO following 25, 75, and 200 mg dosing of a compound disclosed herein under fasting dosing conditions.

FIG. 6 shows a graph illustrating the simulated steady-state AUCₜₐᵤ for the metabolite of a compound disclosed herein after once daily dosing at 25, 75, and 200 mg under fasting dosing conditions.

FIG. 7 shows a graph illustrating the change in body weight after treatment with an α4β7 small molecule inhibitor and a mouse anti p40 antibody in the IL10−/− knock-out mouse colitis model as measured during 21-day treatment.

FIG. 8 shows a graph illustrating the reduction in disease activity in the IL10−/− knock-out mouse colitis model as measured during 21-day treatment with an α4β7 small molecule inhibitor and a mouse anti p40 antibody, as measured by a composite score of stool weight loss, stool frequency, and stool consistency.

FIG. 9 shows a graph illustrating the colonoscopy scores in the IL10−/− knock-out mouse colitis model as measured at the end of the 21-day treatment with an α4β7 small molecule inhibitor and a mouse anti-p40 antibody.

FIG. 10 shows a graph illustrating the histology scores in the IL10−/− knock-out mouse colitis model as measured at the end of the 21-day treatment with an α4β7 small molecule inhibitor and a mouse anti-p40 antibody.

FIG. 11 shows a graph illustrating the percent % of T-helper (TH) 1 cells in colon tissues of mice at 21-days of treatment with an α4β7 small molecule inhibitor and a mouse anti p40 antibody, as measured by flow cytometry.

FIG. 12 shows a graph illustrating the percent % of T-helper (TH) 17 cells in colon tissues of mice the end of the 21-day treatment with an α4β7 small molecule inhibitor and a mouse anti p40 antibody, as measured by flow cytometry.

FIG. 13 shows a graph illustrating the percent % of neutrophil cells in colon tissues of mice at the end of the 21-day treatment with an α4β7 small molecule inhibitor and a mouse anti p40 antibody, as measured by flow cytometry.

FIG. 14 shows a graph illustrating the percent % of T-regulatory (T_{reg}) cells in colon tissues of mice at the end of the 21-day treatment with an α4β7 small molecule inhibitor and a mouse anti p40 antibody as measured by flow cytometry.

FIG. 15 shows a graph illustrating the amount (pg/mL) of IL-1β present in blood of mice at the end of the 21-day treatment with an α4β7 small molecule inhibitor and a mouse anti p40 antibody.

FIG. 16 shows a graph illustrating the amount (pg/mL) of TNF-α present in blood of mice at the end of the 21-day treatment with an α4β7 small molecule inhibitor and a mouse anti p40 antibody.

FIG. 17 shows a graph illustrating the amount (pg/mL) of INF-γ present in blood of mice at the end of the 21-day treatment with an α4β7 small molecule inhibitor and a mouse anti p40 antibody.

FIG. 18 shows a graph illustrating the amount (pg/mL) of IL-6 present in blood of mice at the end of the 21-day treatment with an 487 small molecule inhibitor and a mouse anti p40 antibody.

FIG. 19 shows a graph illustrating the % change in body weight of IL10−/− knock out mouse colitis model as measured during 21-day treatment with an α4β7 small molecule inhibitor and a mouse anti p40 antibody.

FIG. 20 shows a graph illustrating the histology score of IL10−/− knock out mouse colitis model as measured at the end of the 21-day treatment with an α4β7 small molecule inhibitor and a mouse anti p40 antibody.

FIG. 21 shows a graph illustrating the amount (pg/mL) of IL-6 present in blood of mice at the end of the 21-day treatment with an α4β7 small molecule inhibitor and a mouse anti p40 antibody.

FIG. 22 shows a graph illustrating the amount (pg/mL) of IL-1β present in blood of mice at the end of the 21-day treatment with an α4β7 small molecule inhibitor and a mouse anti p40 antibody.

FIG. 23 shows a graph illustrating the percent % of T-helper (TH) 17 cells in colon tissues of mice at the end of the 21-day treatment with an α4β7 small molecule inhibitor and a mouse anti p40 antibody.

FIG. 24 shows a graph illustrating the percent % of T-regulatory (T_{reg}) cells in colon tissues of mice at the end of the 21-day treatment with an α4β7 small molecule inhibitor and a mouse anti p40 antibody.

FIG. 25 shows an illustration of the schematics of the study design for Part 1 of Experiment 4 described herein.

FIG. 26 shows an illustration of the schematics of the study design for Part 2 of Experiment 4 described herein.

FIG. 27 shows an illustration of the schematics of study design for Experiment 5 described herein.

### DETAILED DESCRIPTION

As used in the present specification, the following terms and phrases are generally intended to have the meanings as set forth below, except to the extent that the context in which they are used indicates otherwise.

Unless defined otherwise “a” and “an” mean “one or more.”

As used herein, the term “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Also as used herein, “and/or” refers to and encompasses any and all possible combinations of one or more of the associated listed items, as well as the lack of combinations when interpreted in the alternative (“or”).

As used herein the term “anti-Interleukin-12/Interleukin 23 antibody,” or “anti-IL-12/IL-23 antibody” includes any protein or peptide containing molecule that comprises at least a portion of the immune-globulin molecule, such as but not limited to, at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (Clq) of the classical complement system. At least one complementary determining region (CDR) of a heavy or light chain or a ligand binding portion thereof, a heavy chain constant region, a framework region, or any portion thereof, or at least one portion of an IL-12 and/or IL-23 receptor binding protein, which can be incorporated into an antibody. Such antibodies optionally affect a specific ligand, such as but not limited to where such antibody modulate, decreases, increases antagonizes, agonizes, mitigates, alleviates, clocks, inhibits, abrogates, and/or interferes with at least one IL-12 and/or IL-23 activity or binding, or with IL-12 and/or IL-23 receptor activity or binding, in vitro, in situ and/or in vivo.

The term “antibody,” as used herein, is used in the broadest sense and encompasses various antibody and antibody-like structures that specifically bind to a single antigen or to multiple antigens (e.g., monospecific antibodies, multispecific antibodies, polyepitopic antibodies, etc.), including but not limited to full-length antibodies, antigen-binding fragments, heavy chain antibodies, single-chain antibodies, and higher order variants of single-chain antibodies. Thus, any reference to an antibody should be understood to refer to the antibody in intact form or an antigen-binding fragment unless the context requires otherwise. Preferably, but not necessarily, antibodies useful herein are isolated and can be produced recombinantly.

As used herein, the phrase “biosimilar” with reference to an IL-12 and/or IL-23 inhibitor means a biologic that is highly similar to and has no clinically meaningful differences from an existing biologic medicine (known as a reference product, such as ustekinumab) that is already licensed by the United States Food and Drug Administration (FDA).

The term “pharmaceutical composition,” as used herein, refers t a formulation of a compound and medium generally accepted in the art for the delivery of the biologically active compound to mammals, e.g., humans. Such a medium may include and any pharmaceutically acceptable carriers, diluents, or excipients.

A “mammal” for purposes of treating an infection, refers to any mammal, including humans, domestic and farm animals, research animals, such as mice, rats, and primates, and zoo, sports, or pet animals, such as dogs, cats, cattle, horses, sheep, pigs, goats, rabbits, etc. In particular embodiments, the mammal is human.

As used here in the term “pharmaceutically acceptable carrier, diluent, or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals. Often, the pharmaceutically acceptable carrier is an aqueous pH-buffered solution. Some examples of materials which can serve as pharmaceutically-acceptable carriers, diluents or excipients include: sterile water; buffers, e.g., phosphate-buffered saline; sugars, such as lactose, glucose, trehalose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; amino acids (e.g., charged amino acids, including without limitation, aspartate, asparagine, glutamate, glutamine, histidine, lysine); and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in Remington: The Science and Practice of Pharmacy 20th Ed. (Lippincott, Williams & Wilkins 2003); Loyd V. Allen Jr (Editor), “Remington: The Science and Practice of Pharmacy,” 22nd Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery, 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic, 2012, Woodhead Publishing; and Shire, “Monoclonal Antibodies: Meeting the Challenges in Manufacturing, Formulation, Delivery and Stability of Final Drug Product, 2015, Woodhead Publishing.

The terms “antagonist” and/or “inhibitor,” is used in the broadest sense, and includes any antibody or compound used as an active pharmaceutical ingredient that partially or fully blocks, inhibits, or neutralizes a biological activity of an epitope, polypeptide, or cell that is specifically binds.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In some embodiments, the compounds of the present disclosure can be in the form of a “prodrug.” The term “prodrug” is defined in the pharmaceutical field as a biologically inactive derivative of a drug that upon administration to the human body is converted to the biologically active parent drug according to some chemical or enzymatic pathway. Examples of prodrugs include esterified carboxylic acids.

The terms “individual,” “subject,” and “patient” are used interchangeably herein, and refer to any individual human.

The compounds of the present disclosure can be in the form of a pharmaceutically acceptable salt. The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts, in particular their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine, amino acids, or other bases known to persons skilled in the art. The compounds of the present disclosure which contain one or more basic groups, i.e., groups which can be protonated, can be present and can be used according to the disclosure in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to persons skilled in the art. If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. Acids and bases useful for reaction with an underlying compound to form pharmaceutically acceptable salts (acid addition or base addition salts respectively) are known to one of skill in the art. Similarly, methods of preparing pharmaceutically acceptable salts from an underlying compound (upon disclosure) are known to one of skill in the art and are disclosed in for example, Berge, at al. Journal of Pharmaceutical Science, January 1977 vol. 66, No. 1, and other sources.

Furthermore, compounds disclosed herein may be subject to tautomerism. Where tautomerism, e.g., keto-enol tautomerism, of compounds or their prodrugs may occur, the individual forms, like e.g., the keto and enol form, are each within the scope of the disclosure as well as their mixtures in any ratio. The same applies for stereoisomers, like e.g., enantiomers, cis/trans isomers, diastereomers, conformers and the like.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound.

In certain embodiments, provided are optical isomers, racemates, or other mixtures thereof of the compounds described herein or a pharmaceutically acceptable salt or a mixture thereof. If desired, isomers can be separated by methods well known in the art, e.g., by liquid chromatography. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high-pressure liquid chromatography (HPLC) column.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are non-superimposable mirror images of one another. “Diastereomers” are stereoisomers that have at least two asymmetric atoms, but which are not mirror-images of each other. Compounds disclosed herein and their pharmaceutically acceptable salts may, in some embodiments, include an asymmetric center and may thus give rise to enantiomers, diastereomers, and other stereoisomeric forms that may be defined, in terms of absolute stereochemistry, as (R) or(S) or, as (D) or (L) for amino acids. Some embodiments include all such possible isomers, as well as their racemic and optically pure forms. Optically active (+) and (−), (R) and(S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Compositions provided herein that include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

Any formula or structure given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorus, fluorine and chlorine, such as, but not limited to 2H (deuterium, D), 3H (tritium), 11C, 13C, 14C, 15N, 18F, 31P, 32P, 35S, 36Cl and 125I. Various isotopically labeled compounds of the present disclosure, for example those into which radioactive isotopes such as 3H, 13C and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds disclosed herein, in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g., a human. See, for example, Foster, “Deuterium Isotope Effects in Studies of Drug Metabolism,” Trends Pharmacol. Sci. 5 (12): 524-527 (1984). Such compounds are synthesized by means well known in the art, for example by employing starting materials in which one or more hydrogens have been replaced by deuterium. Deuterium labelled or substituted therapeutic compounds of the disclosure may have beneficial DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An 18F labeled compound may be useful for PET or SPECT studies. The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Furthermore, the present disclosure provides pharmaceutical compositions comprising a compound of the present disclosure, or a prodrug compound thereof, or a pharmaceutically acceptable salt or solvate thereof as active ingredient together with a pharmaceutically acceptable carrier.

As used herein, the term “pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure can encompass any composition made by admixing at least one compound of the present disclosure and a pharmaceutically acceptable carrier.

As used herein, the term “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances are well known in the art (see, e.g., Remington's Pharmaceutical Sciences, Mace Publishing Co., Philadelphia, PA 17th Ed. (1985); and Modern Pharmaceutics, Marcel Dekker, Inc. 3rd Ed. (G. S. Banker & C. T. Rhodes, Eds.).

The terms “therapeutically effective amount” and “effective amount” are used interchangeably and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutically acceptable salt thereof for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

Disclosed herein are methods for treating and/or preventing inflammatory bowel disease (IBD) in a subject in need thereof, the method comprising administering to the subject a therapeutically effective amount of an α4β7 integrin small molecule inhibitor.

Further disclosed herein are methods for treating and/or preventing inflammatory bowel disease (IBD) in a subject in need thereof, comprising administering to the subject a therapeutically effective amount of an α4β7 integrin small molecule inhibitor, wherein the α4β7 integrin small molecule inhibitor is a compound of Formula (I):

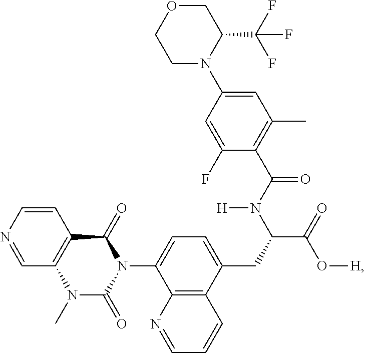

or a pharmaceutically acceptable salt thereof.

Further disclosed herein are methods for treating and/or preventing inflammatory bowel disease (IBD) in a subject in need thereof, comprising administering to the subject a therapeutically effective amount of an α4β7 integrin small molecule inhibitor, wherein the α4β7 integrin small molecule inhibitor is a compound of Formula (II):

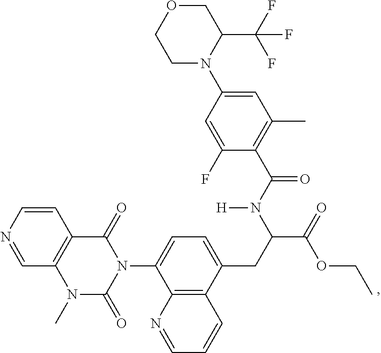 [SMILES: CCOC(=O)C(Cc1ccc(-n2c(=O)c3ccncc3n(C)c2=O)c2ncccc12)NC(=O)c1c(C)cc(N2CCOCC2C(F)(F)F)cc1F]

or a pharmaceutically acceptable salt thereof.

Further disclosed herein are methods for treating and/or preventing inflammatory bowel disease (IBD) in a subject in need thereof, comprising administering to the subject a therapeutically effective amount of an α4β7 integrin small molecule inhibitor, wherein the α4β7 integrin small molecule inhibitor is a compound of Formula (III):

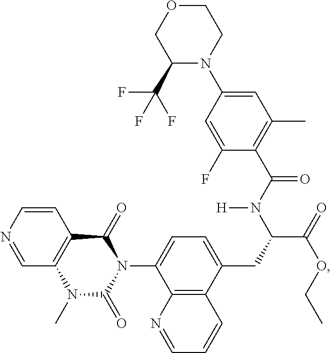

or a pharmaceutically acceptable salt thereof.

Further disclosed herein are methods for treating and/or preventing inflammatory bowel disease (IBD) in a subject in need thereof, comprising administering to the subject a therapeutically effective amount of an α4β7 integrin small molecule inhibitor, wherein the α4β7 integrin small molecule inhibitor is a compound of Formula (IV):

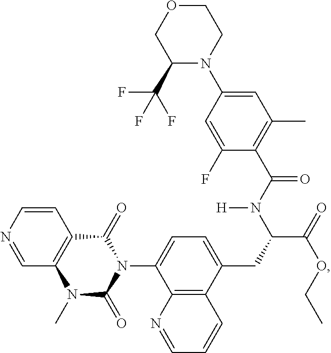

or a pharmaceutically acceptable salt thereof.

Further disclosed herein is a method of treating and/or preventing inflammatory bowel disease (IBD) in a subject in need thereof, comprising administering to the subject an α4β7 integrin small molecule inhibitor, wherein the α4β7 integrin small molecule inhibitor is administered in a dosage of about 10 mg/dose to about 500 mg/dose.

Further disclosed herein is a method of treating and/or preventing inflammatory bowel disease (IBD) in a subject in need thereof, comprising administering to the subject an α4β7 integrin small molecule inhibitor, wherein the α4β7 integrin small molecule inhibitor is administered in a dosage of about 20 mg/dose, or about 75 mg/dose, or about 200 mg/dose.

Further disclosed herein are methods for treating and/or preventing inflammatory bowel disease (IBD) in a subject, comprising administering to the subject (A) a therapeutically effective amount of an α4β7 integrin small molecule inhibitor; and (B) a therapeutically effective amount of an anti-IL-12/IL-23 antibody or an antigen-binding fragment thereof.

Further disclosed herein are methods for treating and/or preventing inflammatory bowel disease (IBD) in a subject, comprising administering to the subject (A) a therapeutically effective amount of an α4β7 integrin small molecule inhibitor, wherein the α4β7 integrin small molecule inhibitor is a compound of Formula (I):

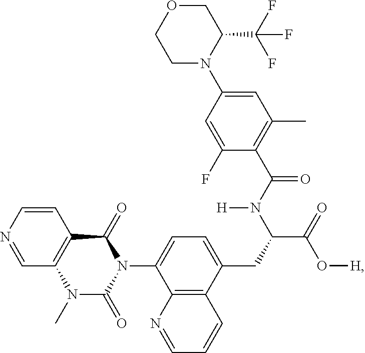

or a pharmaceutically acceptable salt thereof; and (B) a therapeutically effective amount of an anti-IL-12/IL-23 antibody or an antigen-binding fragment thereof.

Further disclosed herein are methods for treating and/or preventing inflammatory bowel disease (IBD) in a subject, comprising administering to the subject: (A) a therapeutically effective amount of an α4β7 integrin small molecule inhibitor, wherein the α4β7 integrin small molecule inhibitor is a compound of Formula (II):

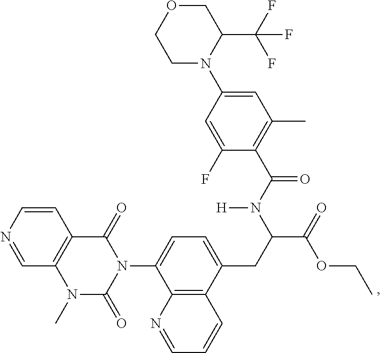 [SMILES: CCOC(=O)C(Cc1ccc(-n2c(=O)c3ccncc3n(C)c2=O)c2ncccc12)NC(=O)c1c(C)cc(N2CCOCC2C(F)(F)F)cc1F]

or a pharmaceutically acceptable salt thereof; and (B) a therapeutically effective amount of an anti-IL-12/IL-23 antibody or an antigen-binding fragment thereof.

Further disclosed herein are methods for treating and/or preventing inflammatory bowel disease (IBD) in a subject, comprising administering to the subject (A) a therapeutically effective amount of an α4β7 integrin small molecule inhibitor, wherein the α4β7 integrin small molecule inhibitor is a compound of Formula (III):

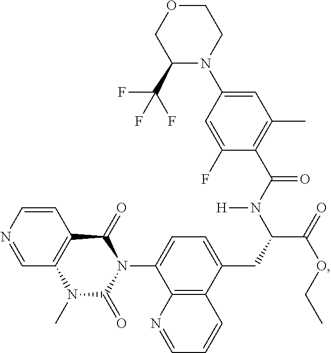

or a pharmaceutically acceptable salt thereof; and (B) a therapeutically effective amount of an anti-IL-12/IL-23 antibody or an antigen-binding fragment thereof.

Further disclosed herein are methods for treating and/or preventing inflammatory bowel disease (IBD) in a subject, comprising administering to the subject (A) a therapeutically effective amount of an α4β7 integrin small molecule inhibitor, wherein the α4β7 integrin small molecule inhibitor is a compound of Formula (IV):

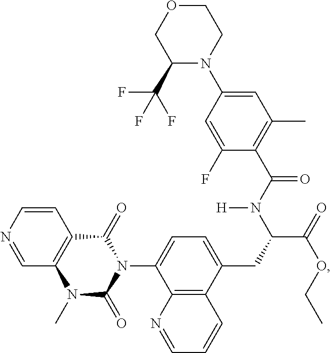

or a pharmaceutically acceptable salt thereof; and (B) a therapeutically effective amount of an anti-IL-12/IL-23 antibody or an antigen-binding fragment thereof.

Further disclosed herein are methods for treating and/or preventing inflammatory bowel disease (IBD) in a subject, comprising administering to the subject (A) a therapeutically effective amount of an α4β7 integrin inhibitor; and (B) a therapeutically effective amount of an anti-IL-12/IL-23 antibody or an antigen-binding fragment thereof, wherein the anti-IL-12/IL-23 antibody or antigen-binding fragment thereof comprises: (I) a heavy chain comprising (a) a heavy chain complementarity-determining region (HCDR) 1 comprising SEQ ID NO: 1; (b) a HCDR2 comprising SEQ ID NO: 2; and (c) a HCDR3 comprising SEQ ID NO: 3; and (II) a light chain comprising (a) a light chain complementarity-determining region (LCDR) 2 comprising SEQ ID NO: 4; (b) a LCDR2 comprising SEQ ID NO: 5; and (c) a LCDR3 comprising SEQ ID NO: 6.

Further disclosed herein are methods for treating and/or preventing inflammatory bowel disease (IBD) in a subject, comprising co-administering to the subject (A) a therapeutically effective amount of an α4β7 integrin inhibitor; and (B) a therapeutically effective amount of an anti-IL-12/IL-23 antibody or an antigen-binding fragment thereof, wherein the anti-IL-12/IL-23 antibody or antigen-binding fragment thereof comprises (I) a heavy chain comprising a heavy chain variable region (VH) sequence that is at least about 90%, 91%, 92%, 93%, 94%, 95%, 96%, 97%, 98%, 99% or 100% identical to the amino acid sequence of SEQ ID NO: 7; and (II) a light chain comprising a light chain variable region (VL) sequence that is at least about 90%, 91%, 92%, 93%, 94%, 95%, 96%, 97%, 98%, 99% or 100% identical to the amino acid sequence of SEQ ID NO: 8.

Further disclosed herein are methods for treating and/or preventing moderately to severely active ulcerative colitis in a patient in need thereof, the method comprising administering to the patient a therapeutically effective amount of an α4β7 small molecule inhibitor in combination with a therapeutically effective amount of an IL-12/IL-23 inhibitor. The presence of moderately to severely active UC can be determined by endoscopy, wherein the patient has a modified Mayo Clinic Score (mMCS) score of 5 to 9, an endoscopic score of at least 2 (determined by central reader), a rectal bleeding score of at least 1, and a stool frequency score of at least 1. As used herein, the term “central reader” means an independent, off-site, blinded reviewer or reading of and endoscopic imaging endpoints.

As used in the present specification, the following terms and phrases are generally intended to have the meanings as set forth below, except to the extent that the context in which they are used indicates otherwise.

Unless defined otherwise “a” and “an” mean “one or more.”

As used herein, the term “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

Also as used herein, “and/or” refers to and encompasses any and all possible combinations of one or more of the associated listed items, as well as the lack of combinations when interpreted in the alternative (“or”).

As used herein the term “anti-Interleukin-12/Interleukin 23 antibody,” or “anti-IL-12/IL-23 antibody” includes any protein or peptide containing molecule that comprises at least a portion of the immune-globulin molecule, such as but not limited to, at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (Clq) of the classical complement system. At least one complementary determining region (CDR) of a heavy or light chain or a ligand binding portion thereof, a heavy chain constant region, a framework region, or any portion thereof, or at least one portion of an IL-12 and/or IL-23 receptor binding protein, which can be incorporated into an antibody. Such antibodies optionally affect a specific ligand, such as but not limited to where such antibody modulate, decreases, increases antagonizes, agonizes, mitigates, alleviates, clocks, inhibits, abrogates, and/or interferes with at least one IL-12 and/or IL-23 activity or binding, or with IL-12 and/or IL-23 receptor activity or binding, in vitro, in situ and/or in vivo.

The term “antibody,” as used herein, is used in the broadest sense and encompasses various antibody and antibody-like structures that specifically bind to a single antigen or to multiple antigens (e.g., monospecific antibodies, multispecific antibodies, polyepitopic antibodies, etc.), including but not limited to full-length antibodies, antigen-binding fragments, heavy chain antibodies, single-chain antibodies, and higher order variants of single-chain antibodies. Thus, any reference to an antibody should be understood to refer to the antibody in intact form or an antigen-binding fragment unless the context requires otherwise. Preferably, but not necessarily, antibodies useful herein are isolated and can be produced recombinantly.

As used herein, the phrase “biosimilar” with reference to an Il-12 and/or IL-23 inhibitor means a biologic that is highly similar to and has no clinically meaningful differences from an existing biologic medicine (known as a reference product, such as ustekinumab) that is already licensed by the United States Food and Drug Administration (FDA).

The term “pharmaceutical composition,” as used herein, refers t a formulation of a compound and medium generally accepted in the art for the delivery of the biologically active compound to mammals, e.g., humans. Such a medium may include and any pharmaceutically acceptable carriers, diluents, or excipients.

A “mammal” for purposes of treating an infection, refers to any mammal, including humans, domestic and farm animals, research animals, such as mice, rats, and primates, and zoo, sports, or pet animals, such as dogs, cats, cattle, horses, sheep, pigs, goats, rabbits, etc. In particular embodiments, the mammal is human.

As used here in the term “pharmaceutically acceptable carrier, diluent, or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals. Often, the pharmaceutically acceptable carrier is an aqueous pH-buffered solution. Some examples of materials which can serve as pharmaceutically-acceptable carriers, diluents or excipients include: sterile water; buffers, e.g., phosphate-buffered saline; sugars, such as lactose, glucose, trehalose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; amino acids (e.g., charged amino acids, including without limitation, aspartate, asparagine, glutamate, glutamine, histidine, lysine); and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described in Remington: The Science and Practice of Pharmacy 20th Ed. (Lippincott, Williams & Wilkins 2003); Loyd V. Allen Jr (Editor), “Remington: The Science and Practice of Pharmacy,” 22nd Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery, 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic, 2012, Woodhead Publishing; and Shire, “Monoclonal Antibodies: Meeting the Challenges in Manufacturing, Formulation, Delivery and Stability of Final Drug Product, 2015, Woodhead Publishing.

The terms “antagonist” and/or “inhibitor,” is used in the broadest sense, and includes any antibody or compound used as an active pharmaceutical ingredient that partially or fully blocks, inhibits, or neutralizes a biological activity of an epitope, polypeptide, or cell that is specifically binds.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In some embodiments, the compounds of the present disclosure can be in the form of a “prodrug.” The term “prodrug” is defined in the pharmaceutical field as a biologically inactive derivative of a drug that upon administration to the human body is converted to the biologically active parent drug according to some chemical or enzymatic pathway. Examples of prodrugs include esterified carboxylic acids.

The terms “individual,” “subject,” and “patient” are used interchangeably herein, and refer to any individual human.

The compounds of the present disclosure can be in the form of a pharmaceutically acceptable salt. The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts, in particular their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine, amino acids, or other bases known to persons skilled in the art. The compounds of the present disclosure which contain one or more basic groups, i.e., groups which can be protonated, can be present and can be used according to the disclosure in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to persons skilled in the art. If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. Acids and bases useful for reaction with an underlying compound to form pharmaceutically acceptable salts (acid addition or base addition salts respectively) are known to one of skill in the art. Similarly, methods of preparing pharmaceutically acceptable salts from an underlying compound (upon disclosure) are known to one of skill in the art and are disclosed in for example, Berge, at al. Journal of Pharmaceutical Science, January 1977 vol. 66, No. 1, and other sources.

Furthermore, compounds disclosed herein may be subject to tautomerism. Where tautomerism, e.g., keto-enol tautomerism, of compounds or their prodrugs may occur, the individual forms, like e.g., the keto and enol form, are each within the scope of the disclosure as well as their mixtures in any ratio. The same applies for stereoisomers, like e.g., enantiomers, cis/trans isomers, diastereomers, conformers and the like.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound.

In certain embodiments, provided are optical isomers, racemates, or other mixtures thereof of the compounds described herein or a pharmaceutically acceptable salt or a mixture thereof. If desired, isomers can be separated by methods well known in the art, e.g., by liquid chromatography. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high-pressure liquid chromatography (HPLC) column.

A “stereoisomer” refers to a compound made up of the same atoms bonded by the same bonds but having different three-dimensional structures, which are not interchangeable. The present invention contemplates various stereoisomers and mixtures thereof and includes “enantiomers,” which refers to two stereoisomers whose molecules are non-superimposable mirror images of one another. “Diastereomers” are stereoisomers that have at least two asymmetric atoms, but which are not mirror-images of each other. Compounds disclosed herein and their pharmaceutically acceptable salts may, in some embodiments, include an asymmetric center and may thus give rise to enantiomers, diastereomers, and other stereoisomeric forms that may be defined, in terms of absolute stereochemistry, as (R) or(S) or, as (D) or (L) for amino acids. Some embodiments include all such possible isomers, as well as their racemic and optically pure forms. Optically active (+) and (−), (R) and(S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Compositions provided herein that include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

Any formula or structure given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorus, fluorine and chlorine, such as, but not limited to 2H (deuterium, D), 3H (tritium), 11C, 13C, 14C, 15N, 18F, 31P, 32P, 35S, 36Cl and 125I. Various isotopically labeled compounds of the present disclosure, for example those into which radioactive isotopes such as 3H, 13C and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds disclosed herein, in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g., a human. See, for example, Foster, “Deuterium Isotope Effects in Studies of Drug Metabolism,” Trends Pharmacol. Sci. 5 (12): 524-527 (1984). Such compounds are synthesized by means well known in the art, for example by employing starting materials in which one or more hydrogens have been replaced by deuterium. Deuterium labelled or substituted therapeutic compounds of the disclosure may have beneficial DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An 18F labeled compound may be useful for PET or SPECT studies. The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Furthermore, the present disclosure provides pharmaceutical compositions comprising a compound of the present disclosure, or a prodrug compound thereof, or a pharmaceutically acceptable salt or solvate thereof as active ingredient together with a pharmaceutically acceptable carrier.

As used herein, the term “pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure can encompass any composition made by admixing at least one compound of the present disclosure and a pharmaceutically acceptable carrier.

As used herein, the term “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances are well known in the art (see, e.g., Remington's Pharmaceutical Sciences, Mace Publishing Co., Philadelphia, PA 17th Ed. (1985); and Modern Pharmaceutics, Marcel Dekker, Inc. 3rd Ed. (G. S. Banker & C. T. Rhodes, Eds.).

The terms “therapeutically effective amount” and “effective amount” are used interchangeably and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutically acceptable salt thereof for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

### α4β7 Small Molecule Inhibitors

The methods disclosed herein comprise administering an α4β7 small molecule inhibitor to a subject. In some embodiments, the subject is a human subject with inflammatory bowel disease (IBD). In some embodiments, the methods and pharmaceutical compositions disclosed herein, comprise administering to a subject a therapeutically effective amount of an α4β7 small molecule inhibitor and another therapeutic agent.

In some embodiments of the methods and pharmaceutical compositions disclosed herein, the α4β7 small molecule inhibitor is selected from a compound of Formula (I), Formula (II), Formula (III), or Formula (IV).

In some embodiments of the methods and pharmaceutical compositions disclosed herein, the α4β7 small molecule inhibitor is a compound having the structure of Formula (I):

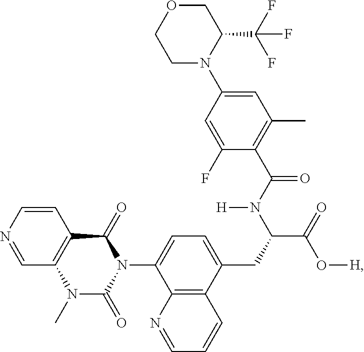

or a pharmaceutically acceptable salt thereof.

In some embodiments of the methods and pharmaceutical compositions disclosed herein, the α4β7 small molecule inhibitor is a compound having the structure of Formula (II):

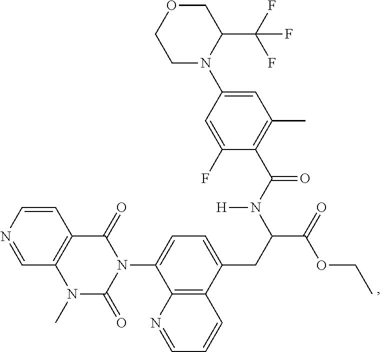 [SMILES: CCOC(=O)C(Cc1ccc(-n2c(=O)c3ccncc3n(C)c2=O)c2ncccc12)NC(=O)c1c(C)cc(N2CCOCC2C(F)(F)F)cc1F]

or a pharmaceutically acceptable salt thereof.

In some embodiments of the methods and pharmaceutical compositions disclosed herein, the α4β7 small molecule inhibitor is a compound having the structure of Formula (III):

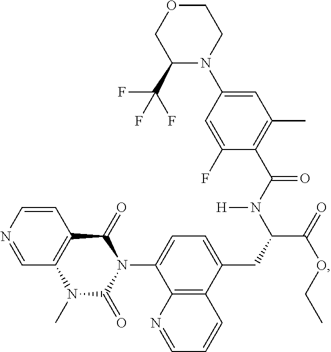

or a pharmaceutically acceptable salt thereof.

In some embodiments, of the methods and pharmaceutical compositions disclosed herein, the α4β7 small molecule inhibitor is a compound having the structure of Formula (IV):

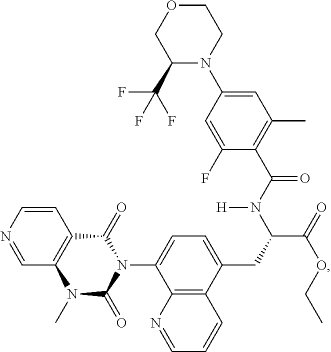

or a pharmaceutically acceptable salt thereof.

The compounds of Formula (I), (II), (III), and (IV) may be synthesized and characterized using methods known to those skilled in the art, such as those described in U.S. Pat. No. 11,116,760, which is hereby incorporated by reference. In one embodiment, the α4β7 small molecule inhibitor is the compound of Formula (I), or a pharmaceutically acceptable salt thereof. In one embodiment, the α4β7 small molecule inhibitor is the compound of Formula (II), or a pharmaceutically acceptable salt thereof. In one embodiment, the α4β7 small molecule inhibitor is the compound of Formula (III), or a pharmaceutically acceptable salt thereof. In one embodiment, the α4β7 small molecule inhibitor is the compound of Formula (IV), or a pharmaceutically acceptable salt thereof.

### IL-12/IL-23 Inhibitors

In some embodiments, the methods and pharmaceutical compositions disclosed herein, comprise administering to a subject a therapeutically effective amount of an IL-12/IL-23 inhibitor. In some embodiments, the methods and pharmaceutical compositions disclosed herein, comprise administering to a subject a therapeutically effective amount of an anti-IL-12/IL-23 antibody. As used herein the term “anti-IL-12/IL-23 antibody” includes antibodies which bind to or interact with at least a portion of IL-12, at least a portion of IL-23, or at least to a portion of both IL-12 and IL-23. For instance, an anti-IL-12/IL-23 antibody disclosed herein binds to or interacts with the p40 subunit that is present in both IL-12 and IL-23. In another instance, an anti-IL-12/IL-23 antibody disclosed herein binds to or interacts with the p19 subunit of IL-23. Examples of anti-IL-12/IL-23 antibodies include, but are not limited to, risankizumab, ustekinumab, guselkumab, brazikumab, mirikizumab, tidarakizumab, briakinumab, and antigen binding fragments thereof.

In some embodiments of the methods disclosed herein, the anti-IL-12/IL-23 antibody is risankizumab or comprises an antigen binding fragment thereof. In some embodiments of the methods disclosed herein, the anti-IL-12/IL-23 antibody is guselkumab or comprises an antigen binding fragment thereof. In some embodiments of the methods disclosed herein, the anti-IL-12/IL-23 antibody is brazikumab or comprises an antigen binding fragment thereof. In some embodiments of the methods disclosed herein, the anti-IL-12/IL-23 antibody is mirikizumab or comprises an antigen binding fragment thereof. In some embodiments of the methods disclosed herein, the anti-IL-12/IL-23 antibody is tidarakizumab or comprises an antigen binding fragment thereof. In some embodiments of the methods disclosed herein, the anti-IL-12/IL-23 antibody is briakinumab or comprises an antigen binding fragment thereof. In some embodiments the anti-IL-12/IL-23 antibody is ustekinumab or comprises an antigen binding fragment thereof.

In some embodiments, the anti-IL-12/IL-23 antibody comprises (A) a heavy chain comprising (I) a heavy chain complementarity-determining region (HCDR) 1 comprising SEQ ID NO: 1; (II) a HCDR2 comprising SEQ ID NO: 2; and (III) a HCDR3 comprising SEQ ID NO: 3; and (B) a light chain comprising (I) a light chain complementarity-determining region (LCDR) 2 comprising SEQ ID NO: 4; (II) a LCDR2 comprising SEQ ID NO: 5; and (III) a LCDR3 comprising SEQ ID NO: 6. In some embodiments, the heavy chain of the anti-IL-12/IL-23 antibody comprises a heavy chain variable region (VH) sequence comprising SEQ ID NO: 7. In some embodiments, the heavy chain of the anti-IL-12/IL-23 antibody comprises a VH sequence that is at least about 90%, 91%, 92%, 93%, 94%, 95%, 96%, 97%, 98%, 99% or 100% identical to the amino acid sequence of SEQ ID NO: 7. In some embodiments, the heavy chain of the anti-IL-12/IL-23 antibody comprises a VH sequence that is at least about 95% identical to the amino acid sequence of SEQ ID NO: 7. In some embodiments, the heavy chain of the anti-IL-12/IL-23 antibody comprises a VH sequence that is at least about 97% identical to the amino acid sequence of SEQ ID NO: 7. In some embodiments, the heavy chain of the anti-IL-12/IL-23 antibody comprises a VH sequence that is at least about 98% identical to the amino acid sequence of SEQ ID NO: 7. In some embodiments, the heavy chain of the anti-IL-12/IL-23 antibody comprises a VH sequence that is at least about 99% identical to the amino acid sequence of SEQ ID NO: 7. In some embodiments, the heavy chain of the anti-IL-12/IL-23 antibody comprises a VH sequence that is about 100% identical to the amino acid sequence of SEQ ID NO: 7. In some embodiments, the heavy chain of the anti-IL-12/IL-23 antibody comprises a full-length heavy chain sequence comprising SEQ ID NO: 9. In some embodiments, the heavy chain of the anti-IL-12/IL-23 antibody comprises a full-length heavy chain sequence that is at least about 90%, 91%, 92%, 93%, 94%, 95%, 96%, 97%, 98%, 99% or 100% identical to the amino acid sequence of SEQ ID NO: 9. In some embodiments, the heavy chain of the anti-IL-12/IL-23 antibody comprises a full-length heavy chain sequence that is at least about 95% identical to the amino acid sequence of SEQ ID NO: 9. In some embodiments, the heavy chain of the anti-IL-12/IL-23 antibody comprises a full-length heavy chain sequence that is at least about 97% identical to the amino acid sequence of SEQ ID NO: 9. In some embodiments, the heavy chain of the anti-IL-12/IL-23 antibody comprises a full-length heavy chain sequence that is at least about 98% identical to the amino acid sequence of SEQ ID NO: 9. In some embodiments, the heavy chain of the anti-IL-12/IL-23 antibody comprises a full-length heavy chain sequence that is at least about 99% identical to the amino acid sequence of SEQ ID NO: 9. In some embodiments, the heavy chain of the anti-IL-12/IL-23 antibody comprises a full-length heavy chain sequence that is 100% identical to the amino acid sequence of SEQ ID NO: 9. In some embodiments, the light chain of the anti-IL-12/IL-23 antibody comprises a light chain variable region (VL) sequence comprising SEQ ID NO: 8. In some embodiments, the light chain of the anti-IL-12/IL-23 antibody comprises a VL sequence that is at least about 90%, 91%, 92%, 93%, 94%, 95%, 96%, 97%, 98%, 99% or 100% identical to the amino acid sequence of SEQ ID NO: 8. In some embodiments, the light chain of the anti-IL-12/IL-23 antibody comprises a VL sequence that is at least about 95% identical to the amino acid sequence of SEQ ID NO: 8. In some embodiments, the light chain of the anti-IL-12/IL-23 antibody comprises a VL sequence that is at least about 97% identical to the amino acid sequence of SEQ ID NO: 8. In some embodiments, the light chain of the anti-IL-12/IL-23 antibody comprises a VL sequence that is at least about 98% identical to the amino acid sequence of SEQ ID NO: 8. In some embodiments, the light chain of the anti-IL-12/IL-23 antibody comprises a VL sequence that is at least about 99% identical to the amino acid sequence of SEQ ID NO: 8. In some embodiments, the light chain of the anti-IL-12/IL-23 antibody comprises a VL sequence that is 100% identical to the amino acid sequence of SEQ ID NO: 8. In some embodiments, the light chain of the anti-IL-12/IL-23 antibody comprises a full-length light chain sequence comprising SEQ ID NO: 10. In some embodiments, the light chain of the anti-IL-12/IL-23 antibody comprises a full-length light chain sequence that is at least about 90%, 91%, 92%, 93%, 94%, 95%, 96%, 97%, 98%, 99% or 100% identical to the amino acid sequence of SEQ ID NO: 10. In some embodiments, the light chain of the anti-IL-12/IL-23 antibody comprises a full-length light chain sequence that is at least about 95% identical to the amino acid sequence of SEQ ID NO: 10. In some embodiments, the light chain of the anti-IL-12/IL-23 antibody comprises a full-length light chain sequence that is at least about 97% identical to the amino acid sequence of SEQ ID NO: 10. In some embodiments, the light chain of the anti-IL-12/IL-23 antibody comprises a full-length light chain sequence that is at least about 98% identical to the amino acid sequence of SEQ ID NO: 10. In some embodiments, the light chain of the anti-IL-12/IL-23 antibody comprises a full-length light chain sequence that is at least about 99% identical to the amino acid sequence of SEQ ID NO: 10. In some embodiments, the light chain of the anti-IL-12/IL-23 antibody comprises a full-length light chain sequence that is 100% identical to the amino acid sequence of SEQ ID NO: 10.

In certain embodiments of the methods disclosed herein the anti-IL-12/IL-23 antibody is ustekinumab. Ustekinumab (Stelara®) may be synthesized and characterized using methods known to those skilled in the art, such as those described in U.S. Pat. No. 6,902,734. Risankizumab (Skyrizi®) may be synthesized and characterized using methods known to those skilled in the art, such as those described in U.S. Pat. No. 8,778,346. Guselkumab (Tremfya®) may be synthesized and characterized using methods known to those skilled in the art, such as those described in U.S. Pat. No. 7,935,344. Brazikumab (AMG139) may be synthesized and characterized using methods known to those skilled in the art, such as those described in U.S. Pat. No. 7,491,391. Mirikizumab (Omvoh™) may be synthesized and characterized using methods known to those skilled in the art, such as those described in U.S. Pat. No. 9,023,358. Tidarkizumab (Actemra®) may be synthesized and characterized using methods known to those skilled in the art, such as those described in U.S. Pat. No. 8,293,883.

TABLE 1
Ustekinumab CDR Sequences

| Description | SEQ ID NO: | Sequence |
|---|---|---|
| CDR-H1 | 1 | TYWLG |
| CDR-H2 | 2 | IMSPVDSDIRYSPSFQG |
| CDR-H3 | 3 | RRPGQGYFDF |
| CDR-L1 | 4 | RASQGISSWLA |
| CDR-L2 | 5 | AASSLQS |
| CDR-L3 | 6 | QQYNIYPYT |

TABLE 2
Ustekinumab Variable Region and Constant Region Sequences

| | SEQ ID | |
|---|---|---|
| Description | NO: | Sequence |
| V_{H} | 7 | EVQLVQSGAEVKKPGESLKISCKGSGYSFTTYWLGWVR |
| | | QMPGKGLDWIGIMSPVDSDIRYSPSFQGQVTMSVDKSIT |
| | | TAYLQWNSLKASDTAMYYCARRRPGQGYFDFWGQGT |
| | | LVTVSS |
| V_{L} | 8 | DIQMTQSPSSLSASVGDRVTITCRASQGISSWLAWYQQK |
| | | PEKAPKSLIYAASSLQSGVPSRFSGSGSGTDFTLTISSLQP |
| | | EDFATYYCQQYNIYPYTFGQGTKLEIKR |
| C_{H} | 9 | SSTKGPSVFPLAPSSKSTSGGTAALGCLVKDYFPEPVTVS |
| | | WNSGALTSGVHTFPAVLQSSGLYSLSSVVTVPSSSLGTQ |
| | | TYICNVNHKPSNTKVDKRVEPKSCDKTHTCPPCPAPELL |
| | | GGPSVFLFPPKPKDTLMISRTPEVTCVVVDVSHEDPEVK |
| | | FNWYVDGVEVHNAKTKPREEQYNSTYRVVSVLTVLHQ |
| | | DWLNGKEYKCKVSNKALPAPIEKTISKAKGQPREPQVY |
| | | TLPPSRDELTKNQVSLTCLVKGFYPSDIAVEWESNGQPE |
| | | NNYKTTPPVLDSDGSFFLYSKLTVDKSRWQQGNVFSCS |
| | | VMHEALHNHYTQKSLSLSPGK |
| C_{L} | 10 | TVAAPSVFIFPPSDEQLKSGTASVVCLLNNFYPREAKVQ |
| | | WKVDNALQSGNSQESVTEQDSKDSTYSLSSTLTLSKAD |
| | | YEKHKVYACEVTHQGLSSPVTKSFNRGEC |
| HC full-length | 11 | EVQLVQSGAEVKKPGESLKISCKGSGYSFTTYWLGWVR |
| | | QMPGKGLDWIGIMSPVDSDIRYSPSFQGQVTMSVDKSIT |
| | | TAYLQWNSLKASDTAMYYCARRRPGQGYFDFWGQGT |
| | | LVTVSSSSTKGPSVFPLAPSSKSTSGGTAALGCLVKDYFP |
| | | EPVTVSWNSGALTSGVHTFPAVLQSSGLYSLSSVVTVPS |
| | | SSLGTQTYICNVNHKPSNTKVDKRVEPKSCDKTHTCPPC |
| | | PAPELLGGPSVFLFPPKPKDTLMISRTPEVTCVVVDVSHE |
| | | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSRDELTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| LC full-length | 12 | DIQMTQSPSSLSASVGDRVTITCRASQGISSWLAWYQQK |
| | | PEKAPKSLIYAASSLQSGVPSRFSGSGSGTDFTLTISSLQP |
| | | EDFATYYCQQYNIYPYTFGQGTKLEIKRTVAAPSVFIFPP |
| | | SDEQLKSGTASVVCLLNNFYPREAKVQWKVDNALQSG |
| | | NSQESVTEQDSKDSTYSLSSTLTLSKADYEKHKVYACE |
| | | VTHQGLSSPVTKSFNRGEC |

In some embodiments, the anti-IL-12/IL-23 antibody is a chimeric, humanized, veneered, or human antibody. In some embodiments, the anti-IL-12/IL-23 antibody is a humanized antibody. In some embodiments, the anti-IL-12/IL-23 antibody comprises an immunoglobulin (Ig) heavy chain selected from gamma (G), alpha (A), delta (D), epsilon (E), or mu (M). In some embodiments, the Ig heavy chain comprises an immunoglobulin gamma (IgG) heavy chain. In some embodiments, the IgG heavy chain is selected from IgG1, IgG2, IgG3, or IgG4. In some embodiments, the IgG heavy chain comprises IgG1. In some embodiments, the IgG heavy chain comprises an IgG1 heavy chain that has up to 10 amino acid substitutions in the constant region. In some embodiments, the IgG heavy chain comprises IgG4. In some embodiments, the IgG heavy chain comprises an IgG4 heavy chain that has up to 10 amino acid substitutions in the constant region. In some embodiments, the heavy chain of the anti-IL-12/IL-23 antibody comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (all numbering according to EU Index). In some embodiments, the anti-IL-12/IL-23 antibody comprises a human IgG1 antibody having one or more substitutions selected from G236D, G237D, P238D, D265A, S267E, P271G, A330R, K322A, and N297A (all numbering according to EU Index). In some embodiments, the anti-IL-12/IL-23 antibody comprises a human IgG4 antibody having one or more substitutions selected from P238D, G237D, P271G, S330R, F234A and L235A (all numbering according to EU Index).

### Doses, Dosing Regimens, and Routes of Administration of the α4β7 Integrin Small Molecule Inhibitor

It is possible for the active ingredient to be administered alone, they may be administered as pharmaceutical formulations or pharmaceutical compositions as described below. As used herein, the term “active ingredient” refers to compounds of Formula (I), (II), (III), and (IV). The formulations, both for veterinary and for human use, of the disclosure comprise at least one active ingredient, together with one or more acceptable carriers, therefor and optionally other therapeutic ingredients. The carrier(s) must be “acceptable” in the sense of being compatible with the other ingredients of the formulation and physiologically innocuous to the recipient thereof.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulation's ranges from about 3 to about 11 but is ordinarily about 7 to 10.

In some embodiments, the method comprises administering the α4β7 integrin small molecule inhibitor at a dose of between about 5 mg to about 2000 mg, between about 5 mg to about 1800 mg, between about 5 mg to about 1600 mg, between about 5 mg to about 1500 mg, between about 5 mg to about 1400 mg, between about 5 mg to about 1200 mg, between about 5 mg to about 1000 mg, between about 5 mg to about 800 mg, between about 5 mg to about 700 mg, between about 5 mg to about 600 mg, between about 5 mg to about 500 mg, between about 5 mg to about 400 mg, between about 5 mg to about 300 mg, between about 5 mg to about 250 mg, between about 5 mg to about 200 mg, between 10 mg to about 2000 mg, between about 10 mg to about 1800 mg, between about 10 mg to about 1600 mg, between about 10 mg to about 1500 mg, between about 10 mg to about 1400 mg, between about 10 mg to about 1200 mg, between about 10 mg to about 1000 mg, between about 10 mg to about 800 mg, between about 10 mg to about 700 mg, between about 10 mg to about 600 mg, between about 10 mg to about 500 mg, between about 10 mg to about 400 mg, between about 10 mg to about 300 mg, between about 10 mg to about 250 mg, between about 10 mg to about 200 mg, between about 15 mg to about 2000 mg, between about 15 mg to about 1800 mg, between about 15 mg to about 1600 mg, between about 15 mg to about 1500 mg, between about 15 mg to about 1400 mg, between about 15 mg to about 1200 mg, between about 15 mg to about 1000 mg, between about 15 mg to about 800 mg, between about 15 mg to about 700 mg, between about 5 mg to about 600 mg, between about 15 mg to about 500 mg, between about 15 mg to about 400 mg, between about 15 mg to about 300 mg, between about 15 mg to about 250 mg, between about 15 mg to about 200 mg, between about 20 mg to about 2000 mg, between about 20 mg to about 1800 mg, between about 20 mg to about 1600 mg, between about 20 mg to about 1500 mg, between about 20 mg to about 1400 mg, between about 20 mg to about 1200 mg, between about 20 mg to about 1000 mg, between about 20 mg to about 800 mg, between about 20 mg to about 700 mg, between about 20 mg to about 600 mg, between about 20 mg to about 500 mg, between about 20 mg to about 400 mg, between about 20 mg to about 300 mg, between about 20 mg to about 250 mg, or between about 20 mg to about 200 mg. In some embodiments, the method comprises administering the α4β7 integrin small molecule inhibitor at a dose of between about 20 mg to about 1000 mg. In some embodiments, the method comprises administering the α4β7 integrin small molecule inhibitor at a dose of between about 20 mg to about 500 mg. In some embodiments, the method comprises administering the α4β7 integrin small molecule inhibitor at a dose of about 25 mg. In some embodiments, the method comprises administering the α4β7 integrin small molecule inhibitor at a dose of about 75 mg. In some embodiments, the method comprises administering the α4β7 integrin small molecule inhibitor at a dose of about 100 mg. In some embodiments, the method comprises administering the α4β7 integrin small molecule inhibitor at a dose of about 200 mg.

In some embodiments, the α4β7 integrin small molecule inhibitor is administered by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, the α4β7 integrin small molecule inhibitor is administered orally.

In some embodiments, the α4β7 small molecule inhibitor is dosed daily or every 2, 3, 4, 5, 6, 7, 8, 9, 10, 11, 12, 13, 14 or more days. In some embodiments, the α4β7 small molecule inhibitor is dosed 1, 2, 3, 4, 5, 6, 7, 8, 9, 10, 11, 12, 13, 14 or more times a day. In some embodiments, the α4β7 small molecule inhibitor is dosed 1 or more times a day every 1, 2, 3, 4, 5, 6, 7, 8, 9, 10, 11, 12, 13, 14 or more days. In some embodiments, the α4β7 small molecule inhibitor is dosed once daily.

In some embodiments, the α4β7 small molecule inhibitor is dosed daily for 12 weeks. In some embodiments, the α4β7 small molecule inhibitor is dosed daily for 52 weeks. In some embodiments, the α4β7 small molecule inhibitor is dosed daily for 76 weeks. In some embodiments, the α4β7 small molecule inhibitor is dosed 1, 2, 3, 4, 5, 6, 7, 8, 9, 10, 11, 12, 13, 14 or more times a day. In some embodiments, the α4β7 small molecule inhibitor is dosed 1 or more times a day every 12 weeks. In some embodiments, the α4β7 small molecule inhibitor is dosed 1 or more times a day every 52 weeks. In some embodiments, the α4β7 small molecule inhibitor is dosed once daily. In some embodiments, the α4β7 small molecule inhibitor is dosed 1 or more times a day every 76 weeks.

In some embodiments, the α4β7 integrin small molecule inhibitor is provided in a solid dosage form.

In some embodiments, the α4β7 integrin small molecule inhibitor is administered under fasting conditions. In some embodiments, the fasting conditions means the subject does not consume food within 4 hours before administration of the α4β7 integrin small molecule inhibitor and within 2 hours after administration of the α4β7 integrin small molecule inhibitor. In some embodiments, the subject does not consume iron or calcium within 3 hours after administration of the α4β7 integrin small molecule inhibitor.

### Doses, Dosing Regimens, and Routes of Administration of the IL-12/IL-23 Inhibitor

In some embodiments, the methods disclosed herein comprise administering a first dose (an induction dose) of the anti-IL-12/IL-23 antibody from between about 100 mg to about 2000 mg, between about 100 mg to about 1800 mg, between about 100 mg to about 1600 mg, between about 100 mg to about 1500 mg, between about 100 mg to about 1400 mg, between about 100 mg to about 1200 mg, between about 100 mg to about 1000 mg, between about 100 mg to about 800 mg, between about 100 mg to about 700 mg, between about 100 mg to about 600 mg, between about 150 mg to about 2000 mg, between about 150 mg to about 1800 mg, between about 150 mg to about 1600 mg, between about 150 mg to about 1500 mg, between about 150 mg to about 1400 mg, between about 150 mg to about 1200 mg, between about 150 mg to about 1000 mg, between about 150 mg to about 800 mg, between about 150 mg to about 700 mg, between about 150 mg to about 600 mg, between about 200 mg to about 2000 mg, between about 200 mg to about 1800 mg, between about 200 mg to about 1600 mg, between about 200 mg to about 1500 mg, between about 200 mg to about 1400 mg, between about 200 mg to about 1200 mg, between about 200 mg to about 1000 mg, between about 200 mg to about 800 mg, between about 200 mg to about 700 mg, between about 200 mg to about 600 mg. In some embodiments, the method comprises administering a first dose (an induction dose) from between about 200 mg to about 600 mg of the anti-IL-12/IL-23 antibody. In some embodiments, the method comprises administering a first dose (an induction dose) from between about 260 mg to about 520 mg of the anti-IL-12/IL-23 antibody. In some embodiments, the induction dose is about 260 mg of the anti-IL-12/IL-23 antibody. In some embodiments, the induction dose is about 390 mg of the anti-IL-12/IL-23 antibody. In some embodiments, the induction dose is about 520 mg of the anti-IL-12/IL-23 antibody. In some embodiments, the induction dose is determined by body weight (kg) of the subject. In some embodiments, the induction dose is (A) about 260 mg of the anti-IL-12/IL-23 antibody for subjects weighing less than or equal to 55 kg; (B) about 390 mg of the anti-IL-12/IL-23 antibody for subjects weighing greater than 55 kg, but less than or equal to 85 kg; or (C) about 520 mg of the anti-IL-12/IL-23 antibody for subjects weighing greater than 85 kg. In some embodiments, the induction dose is about 6.0 mg of the anti-IL-12/IL-23 antibody per kg of body weight. In some embodiments, the induction dose of the anti-IL-12/IL-23 antibody is administered on the same day as the first dose of the α4β7 integrin small molecule inhibitor. In some embodiments, the administration of the induction dose of the anti-IL-12/IL-23 antibody is by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, the administration of the induction dose of the anti-IL-12/IL-23 antibody is by intravenous (IV) injection.

In some embodiments, the method further comprises administering 1, 2, 3, 4, 5, 6, 7, 8, 9, 10 or more maintenance doses of the anti-IL-12/IL-23 antibody. In some embodiments, at least one maintenance dose is between about 10 mg to about 200 mg of the anti-IL-12/IL-23 antibody. In some embodiments, at least one maintenance dose is about 45 mg of the anti-IL-12/IL-23 antibody. In some embodiments, at least one maintenance dose is about 90 mg of the anti-IL-12/IL-23 antibody.

In some embodiments, at least one maintenance dose is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks after the induction dose. In some embodiments, at least one maintenance dose is administered at least 8, 16, 24, 32, 40, or 48 weeks after the induction dose.

In some embodiments, at least 2 maintenance doses are administered at least 1, 2, 3, 4, 5, 6, 7, 8, 9, 10, 11, 12, 13, 14, 15 or more weeks apart. In some embodiments, at least 2 maintenance doses are administered at least 8 or more weeks apart. In some embodiments, at least 2 maintenance doses are administered at least 12 or more weeks apart. In some embodiments, at least 2 maintenance doses are administered every 1, 2, 3, 4, 5, 6, 7, 8, 9, 10, 11, 12, 13, 14, 15 or more weeks. In some embodiments, at least 2 maintenance doses are administered at least every 8 weeks. In some embodiments, at least 2 maintenance doses are administered at least every 12 weeks.

In some embodiments, the method further comprises administering 1, 2, 3, 4, 5, 6 or more maintenance doses of about 90 mg of the anti-IL-12/IL-23 antibody at 8, 16, 24, 32, 40, and 48 weeks after the induction dose. In some embodiments, the method comprises administering a maintenance dose of about 90 mg of the anti-IL-12/IL-23 antibody at week 8. In some embodiments, the method further comprises administering a maintenance dose of about 90 mg of the anti-IL-12/IL-23 antibody at week 16. In some embodiments, the method further comprises administering a maintenance dose of 90 mg of the anti-IL-12/IL-23 antibody at week 24. In some embodiments, the method further comprises administering a maintenance dose of 90 mg of the anti-IL-12/IL-23 antibody at week 32. In some embodiments, the method further comprises administering a maintenance dose of 90 mg of the anti-IL-12/IL-23 antibody at week 40. In some embodiments, the method further comprises administering a maintenance dose of 90 mg of the anti-IL-12/IL-23 antibody at week 48. In some embodiments, the method further comprises administering a maintenance dose of 90 mg of the anti-IL-12/IL-23 antibody at week 52. In some embodiments, the method further comprises administering a maintenance dose of 90 mg of the anti-IL-12/IL-23 antibody at week 8, at week 16, at week 24, at week 32 at week 40 and at week 48. In some embodiments, the dose of 90 mg of the anti-IL-12/IL-23 antibody is administered by subcutaneous injection.

In some embodiments, the method comprises treating the subject for at least 12 weeks, at least 24 weeks, at least 32 weeks, at least 40 weeks, at least 48 weeks, at least 52 weeks, at least 76 weeks, or longer. In some embodiments, the method comprises treating the subject for at least 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or longer. In some embodiments, the method comprises treating the subject for at least 1 year, 2 years, 3 years, 4 years, 5 years, 6 years, 7 years, 8 years, 9 years, 10 years, 11 years, 12 years, or longer.

In some embodiments, the administration of the maintenance dose of the anti-IL-12/IL-23 antibody is by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, at least one maintenance dose of the anti-IL-12/IL-23 antibody is administered by subcutaneous (SC) injection.

In some embodiments, at least one maintenance dose is administered on the same day as one or more doses of the α4β7 integrin small molecule inhibitor.

In some embodiments, a method for treating and/or preventing ulcerative colitis (UC) comprises administering a first dose (e.g., an induction dose) from between about 260 to about 520 mg of the anti-IL-12/IL-23 antibody. In some embodiments, a method for treating and/or preventing UC comprises administering a first dose (e.g., an induction dose) of about 260 mg of the anti-IL-12/IL-23 antibody. In some embodiments, a method for treating and/or preventing UC comprises administering a first dose (e.g., an induction dose) of about 390 mg of the anti-IL-12/IL-23 antibody. In some embodiments, a method for treating and/or preventing UC comprises administering a first dose (e.g., an induction dose) of about 520 mg of the anti-IL-12/IL-23 antibody. In some embodiments, a method for treating and/or preventing UC comprises administering an induction dose of about 6.0 mg of the IL-12/IL-23 antibody per kg of body weight of the subject. In some embodiments, the administration of the induction dose of the anti-IL-12/IL-23 antibody is by intravenous (IV) injection.

### Subject Characteristics

Disclosed herein are methods for treating inflammatory bowel disease (IBD) or uses of the α4β7 integrin small molecule inhibitor alone or in combination with an IL-12/IL-23 inhibitor in the manufacture of a medicament for the of IBD. In some embodiments, the IBD is ulcerative colitis (UC). In some embodiments, the UC is moderately to severely active UC. In some embodiments, the subject has a received prior treatment of one or more conventional or advanced therapies for UC.

In some embodiments, the IBD is Crohn's disease.

In some embodiments, the subject is a human. In some embodiments, the subject does not suffer from chronic or active infections. In some embodiments, the subject does not have a predisposition to infections.

In some embodiments, the subject has one or more of the following baseline laboratory parameters determined by endoscopy prior to treatment (a) a total mMCS of 5 to 9 points; (b) an endoscopic sub-score of ≥2; (c) a rectal bleeding score ≥1; and (d) a stool frequency score of ≥ 1.

In some embodiments, the subject has one or more of the following laboratory parameters at a baseline timepoint prior to treatment (a) aspartate aminotransferase (AST) and alanine aminotransferase (ALT) and total bilirubin≤2× upper limit of normal (ULN); (b) total bilirubin≤2×ULN; (c) alkaline phosphatase <3×ULN; (d) estimated glomerular filtration rate (eGFR)>60 mL/min.; (e) hemoglobin ≥8 g/dL (≥80 g/L); (f) absolute neutrophil count ≥1.5×10³/uL (≥1.5 GI/L); (g) platelets ≥100×10³/uL (≥100 GI/L); (h) white blood cells ≥3×10³/uL (≥3 GI/L); and (i) absolute lymphocyte count ≥0.75×10³/uL (≥0.75 GI/L).

In some embodiments, prior to treatment means prior to administration of the first dose of the α4β7 integrin small molecule inhibitor or prior to co-administration of the first dose of the α4β7 integrin small molecule inhibitor and the first dose of the anti-IL-12/IL-23 antibody.

### Treatment and Monitoring of IBD

Disclosed herein are methods for treating IBD. In some embodiments, treating the IBD results in corticosteroid-free clinical remission of the IBD. In some embodiments, treating the IBD results in clinical remission of the IBD. In some embodiments, treating the IBD results in mucosal healing. In some embodiments, treating the IBD means reducing the severity of the IBD.

In some embodiments, the method further comprises monitoring the subject after administration of one or more doses the α4β7 integrin small molecule inhibitor. In some embodiments, monitoring the subject comprises obtaining one or more biological samples from the subject to determine if the patient has a clinical response. In some embodiments, one or more biological samples are obtained from the subject at least 6, 7, 8, 9, 10, 11, 12, 13, 14, 15, 16, 17, 18, 19, 20, 21, 22, 23, 24, 25, 26, 27, 28, 29, 30 or more weeks after administration of the first dose of the α4β7 integrin small molecule inhibitor. In some embodiments, one or more biological samples are obtained from the subject at least 12 weeks after administration of the first dose of the α4β7 integrin small molecule inhibitor. In some embodiments, one or more biological samples are obtained from the subject at least 52 weeks after administration of the first dose of the α4β7 integrin small molecule inhibitor. In some embodiments, one or more biological samples are obtained from the subject at least 76 weeks after administration of the first dose of the α4β7 integrin small molecule inhibitor. In some embodiments, a clinical response is defined as a decrease from baseline in the mMCS of ≥2 points and at least a 30% reduction from baseline and/or a decrease in rectal bleeding subscore of ≥1 from baseline or an absolute rectal bleeding subscore of 0 or 1.

Disclosed herein is a method of determining clinical responsiveness of a subject with moderate to severe UC to an α4β7 integrin small molecule inhibitor and an anti-IL-12/IL-23 antibody combination therapy, comprising (a) detecting a baseline mMCS of 5 to 9 points in a biological sample obtained from the subject and a rectal bleeding score of ≥1 prior to a first α4β7 integrin small molecule inhibitor administration; (b) administering to the subject a therapeutically effective amount of an α4β7 integrin small molecule inhibitor; c) determining one or more of a treatment mMSC score and a treatment rectal bleeding score from the subject; and (d) generating a clinical response determination based upon one or more of a comparison of the baseline mMCS score to the treatment mMCS score and a comparison of the baseline rectal bleeding subscore to the treatment rectal bleeding subscore. In some embodiments, the baseline mMCS score is 5 to 9 points. In some embodiments, the baseline rectal bleeding subscore is ≥1. In some embodiments, the clinical response determination is a positive clinical response characterized by a treatment mMCS score decrease of ≥2 points and a reduction of at least 30% from the baseline mMCS. In still further embodiments, the clinical response determination is a positive clinical response characterized by a treatment rectal bleeding subscore decrease of ≥1 compared to the baseline rectal bleeding score or an absolute rectal bleeding subscore of 0 or 1. Disclosed herein is a method of determining clinical responsiveness of a subject with moderate to severe UC to an α4β7 integrin small molecule inhibitor, comprising (a) detecting a baseline mMCS of 5 to 9 points in a biological sample obtained from the subject and a rectal bleeding score of ≥1 prior to a first α4β7 integrin small molecule inhibitor administration; (b) administering to the subject a therapeutically effective amount of an α4β7 integrin small molecule inhibitor; c) determining one or more of a treatment mMSC score and a treatment rectal bleeding score from the subject; and (d) generating a clinical response determination based upon one or more of a comparison of the baseline mMCS score to the treatment mMCS score and a comparison of the baseline rectal bleeding subscore to the treatment rectal bleeding subscore. In some embodiments, the baseline mMCS score is 5 to 9 points. In some embodiments, the baseline rectal bleeding subscore is ≥1. In some embodiments, the clinical response determination is a positive clinical response characterized by a treatment mMCS score decrease of ≥2 points and a reduction of at least 30% from the baseline mMCS. In still further embodiments, the clinical response determination is a positive clinical response characterized by a treatment rectal bleeding subscore decrease of ≥1 compared to the baseline rectal bleeding score or an absolute rectal bleeding subscore of 0 or 1.

### Pharmaceutical Compositions

The pharmaceutical compositions of the disclosure comprise an effective amount of an α4β7 small molecule inhibitor selected from the group consisting of a compound of Formula (I), (II), (III), or (IV) or a pharmaceutically acceptable salt thereof.

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring, and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

### Exemplary Methods and Uses

Disclosed herein is a method for treating and/or preventing inflammatory bowel disease (IBD) in a subject in need thereof, comprising administering to the subject a therapeutically effective amount of an α4β7 integrin small molecule inhibitor. In some embodiments, the α4β7 integrin small molecule inhibitor is a compound of Formula (I):

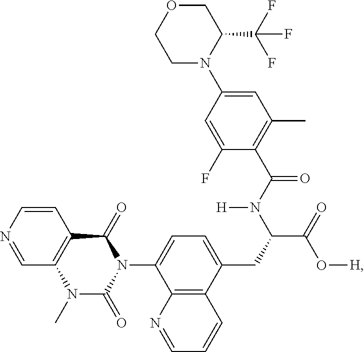

or a pharmaceutically acceptable salt thereof. In some embodiments, the α4β7 integrin small molecule inhibitor is a compound of Formula (II):

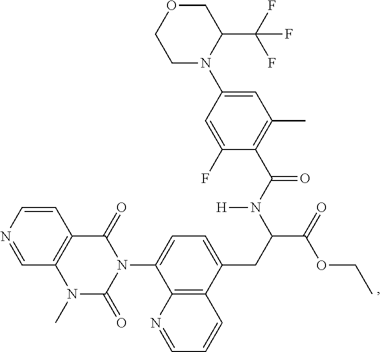 [SMILES: CCOC(=O)C(Cc1ccc(-n2c(=O)c3ccncc3n(C)c2=O)c2ncccc12)NC(=O)c1c(C)cc(N2CCOCC2C(F)(F)F)cc1F]

or a pharmaceutically acceptable salt thereof. In some embodiments, the α4β7 integrin small molecule inhibitor is a compound of Formula (III):

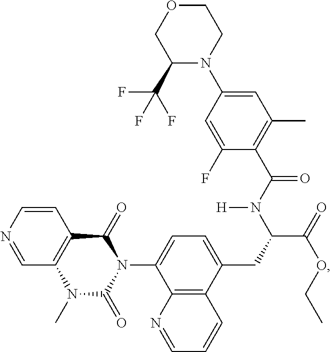

or a pharmaceutically acceptable salt thereof. In some embodiments, the α4β7 integrin small molecule inhibitor is a compound of Formula (IV):

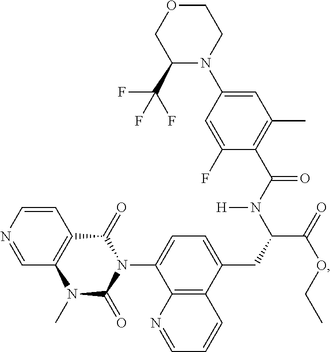

or a pharmaceutically acceptable salt thereof. In some embodiments, the method further comprises administering an anti-IL-12/IL-23 antibody. In some embodiments, the anti-IL-12/IL-23 antibody is selected from risankizumab, ustekinumab, guselkumab, brazikumab, mirikizumab, tidarakizumab, briakinumab, and an antigen binding fragment thereof. In some embodiments, the anti-IL-12/IL-23 antibody is ustekinumab or an antigen binding fragment thereof. In some embodiments, the anti-IL-12/IL-23 antibody comprises (A) a heavy chain comprising (I) a heavy chain complementarity-determining region (HCDR) 1 comprising SEQ ID NO: 1; (II) a HCDR2 comprising SEQ ID NO: 2; and (III) a HCDR3 comprising SEQ ID NO: 3; and (B) a light chain comprising (I) a light chain complementarity-determining region (LCDR) 2 comprising SEQ ID NO: 4; (II) a LCDR2 comprising SEQ ID NO: 5; and (III) a LCDR3 comprising SEQ ID NO: 6. In some embodiments, the heavy chain of the anti-IL-12/IL-23 antibody comprises a heavy chain variable region (VH) sequence comprising SEQ ID NO: 7. In some embodiments, the heavy chain of the anti-IL-12/IL-23 antibody comprises a VH sequence that is at least about 90%, 91%, 92%, 93%, 94%, 95%, 96%, 97%, 98%, 99% or 100% identical to the amino acid sequence of SEQ ID NO: 7. In some embodiments, the heavy chain of the anti-IL-12/IL-23 antibody comprises a VH sequence that is at least about 95% identical to the amino acid sequence of SEQ ID NO: 7. In some embodiments, the heavy chain of the anti-IL-12/IL-23 antibody comprises a VH sequence that is at least about 97% identical to the amino acid sequence of SEQ ID NO: 7. In some embodiments, the heavy chain of the anti-IL-12/IL-23 antibody comprises a VH sequence that is at least about 98% identical to the amino acid sequence of SEQ ID NO: 7. In some embodiments, the heavy chain of the anti-IL-12/IL-23 antibody comprises a VH sequence that is at least about 99% identical to the amino acid sequence of SEQ ID NO: 7. In some embodiments, the heavy chain of the anti-IL-12/IL-23 antibody comprises a VH sequence that is about 100% identical to the amino acid sequence of SEQ ID NO: 7. In some embodiments, the heavy chain of the anti-IL-12/IL-23 antibody comprises a full-length heavy chain sequence comprising SEQ ID NO: 9. In some embodiments, the heavy chain of the anti-IL-12/IL-23 antibody comprises a full-length heavy chain sequence that is at least about 90%, 91%, 92%, 93%, 94%, 95%, 96%, 97%, 98%, 99% or 100% identical to the amino acid sequence of SEQ ID NO: 9. In some embodiments, the heavy chain of the anti-IL-12/IL-23 antibody comprises a full-length heavy chain sequence that is at least about 95% identical to the amino acid sequence of SEQ ID NO: 9. In some embodiments, the heavy chain of the anti-IL-12/IL-23 antibody comprises a full-length heavy chain sequence that is at least about 97% identical to the amino acid sequence of SEQ ID NO: 9. In some embodiments, the heavy chain of the anti-IL-12/IL-23 antibody comprises a full-length heavy chain sequence that is at least about 98% identical to the amino acid sequence of SEQ ID NO: 9. In some embodiments, the heavy chain of the anti-IL-12/IL-23 antibody comprises a full-length heavy chain sequence that is at least about 99% identical to the amino acid sequence of SEQ ID NO: 9. In some embodiments, the heavy chain of the anti-IL-12/IL-23 antibody comprises a full-length heavy chain sequence that is 100% identical to the amino acid sequence of SEQ ID NO: 9. In some embodiments, the light chain of the anti-IL-12/IL-23 antibody comprises a light chain variable region (VL) sequence comprising SEQ ID NO: 8. In some embodiments, the light chain of the anti-IL-12/IL-23 antibody comprises a VL sequence that is at least about 90%, 91%, 92%, 93%, 94%, 95%, 96%, 97%, 98%, 99% or 100% identical to the amino acid sequence of SEQ ID NO: 8. In some embodiments, the light chain of the anti-IL-12/IL-23 antibody comprises a VL sequence that is at least about 95% identical to the amino acid sequence of SEQ ID NO: 8. In some embodiments, the light chain of the anti-IL-12/IL-23 antibody comprises a VL sequence that is at least about 97% identical to the amino acid sequence of SEQ ID NO: 8. In some embodiments, the light chain of the anti-IL-12/IL-23 antibody comprises a VL sequence that is at least about 98% identical to the amino acid sequence of SEQ ID NO: 8. In some embodiments, the light chain of the anti-IL-12/IL-23 antibody comprises a VL sequence that is at least about 99% identical to the amino acid sequence of SEQ ID NO: 8. In some embodiments, the light chain of the anti-IL-12/IL-23 antibody comprises a VL sequence that is 100% identical to the amino acid sequence of SEQ ID NO: 8. In some embodiments, the light chain of the anti-IL-12/IL-23 antibody comprises a full-length light chain sequence comprising SEQ ID NO: 10. In some embodiments, the light chain of the anti-IL-12/IL-23 antibody comprises a full-length light chain sequence that is at least about 90%, 91%, 92%, 93%, 94%, 95%, 96%, 97%, 98%, 99% or 100% identical to the amino acid sequence of SEQ ID NO: 10. In some embodiments, the light chain of the anti-IL-12/IL-23 antibody comprises a full-length light chain sequence that is at least about 95% identical to the amino acid sequence of SEQ ID NO: 10. In some embodiments, the light chain of the anti-IL-12/IL-23 antibody comprises a full-length light chain sequence that is at least about 97% identical to the amino acid sequence of SEQ ID NO: 10. In some embodiments, the light chain of the anti-IL-12/IL-23 antibody comprises a full-length light chain sequence that is at least about 98% identical to the amino acid sequence of SEQ ID NO: 10. In some embodiments, the light chain of the anti-IL-12/IL-23 antibody comprises a full-length light chain sequence that is at least about 99% identical to the amino acid sequence of SEQ ID NO: 10. In some embodiments, the light chain of the anti-IL-12/IL-23 antibody comprises a full-length light chain sequence that is 100% identical to the amino acid sequence of SEQ ID NO: 10. In some embodiments, the anti-IL-12/IL-23 antibody is a chimeric, humanized, veneered, or human antibody. In some embodiments, the anti-IL-12/IL-23 antibody is a humanized antibody. In some embodiments, the anti-IL-12/IL-23 antibody comprises an immunoglobulin (Ig) heavy chain selected from gamma (G), alpha (A), delta (D), epsilon (E), or mu (M). In some embodiments, the Ig heavy chain comprises an immunoglobulin gamma (IgG) heavy chain. In some embodiments, the IgG heavy chain is selected from IgG1, IgG2, IgG3, or IgG4. In some embodiments, the IgG heavy chain comprises IgG1. In some embodiments, the IgG heavy chain comprises an IgG1 heavy chain that has up to 10 amino acid substitutions in the constant region. In some embodiments, the IgG heavy chain comprises IgG4. In some embodiments, the IgG heavy chain comprises an IgG4 heavy chain that has up to 10 amino acid substitutions in the constant region. In some embodiments, the heavy chain of the anti-IL-12/IL-23 antibody comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (all numbering according to EU Index). In some embodiments, the anti-IL-12/IL-23 antibody comprises a human IgG1 antibody having one or more substitutions selected from G236D, G237D, P238D, D265A, S267E, P271G, A330R, K322A, and N297A (all numbering according to EU Index). In some embodiments, the anti-IL-12/IL-23 antibody comprises a human IgG4 antibody having one or more substitutions selected from P238D, G237D, P271G, S330R, F234A and L235A (all numbering according to EU Index). In some embodiments, the method comprises administering the α4β7 integrin small molecule inhibitor at a dose of between about 5 mg to about 2000 mg, between about 5 mg to about 1800 mg, between about 5 mg to about 1600 mg, between about 5 mg to about 1500 mg, between about 5 mg to about 1400 mg, between about 5 mg to about 1200 mg, between about 5 mg to about 1000 mg, between about 5 mg to about 800 mg, between about 5 mg to about 700 mg, between about 5 mg to about 600 mg, between about 5 mg to about 500 mg, between about 5 mg to about 400 mg, between about 5 mg to about 300 mg, between about 5 mg to about 250 mg, between about 5 mg to about 200 mg, between 10 mg to about 2000 mg, between about 10 mg to about 1800 mg, between about 10 mg to about 1600 mg, between about 10 mg to about 1500 mg, between about 10 mg to about 1400 mg, between about 10 mg to about 1200 mg, between about 10 mg to about 1000 mg, between about 10 mg to about 800 mg, between about 10 mg to about 700 mg, between about 10 mg to about 600 mg, between about 10 mg to about 500 mg, between about 10 mg to about 400 mg, between about 10 mg to about 300 mg, between about 10 mg to about 250 mg, between about 10 mg to about 200 mg, between about 15 mg to about 2000 mg, between about 15 mg to about 1800 mg, between about 15 mg to about 1600 mg, between about 15 mg to about 1500 mg, between about 15 mg to about 1400 mg, between about 15 mg to about 1200 mg, between about 15 mg to about 1000 mg, between about 15 mg to about 800 mg, between about 15 mg to about 700 mg, between about 5 mg to about 600 mg, between about 15 mg to about 500 mg, between about 15 mg to about 400 mg, between about 15 mg to about 300 mg, between about 15 mg to about 250 mg, between about 15 mg to about 200 mg, between about 20 mg to about 2000 mg, between about 20 mg to about 1800 mg, between about 20 mg to about 1600 mg, between about 20 mg to about 1500 mg, between about 20 mg to about 1400 mg, between about 20 mg to about 1200 mg, between about 20 mg to about 1000 mg, between about 20 mg to about 800 mg, between about 20 mg to about 700 mg, between about 20 mg to about 600 mg, between about 20 mg to about 500 mg, between about 20 mg to about 400 mg, between about 20 mg to about 300 mg, between about 20 mg to about 250 mg, or between about 20 mg to about 200 mg. In some embodiments, the method comprises administering the α4β7 integrin small molecule inhibitor at a dose of between about 20 mg to about 1000 mg. In some embodiments, the method comprises administering the α4β7 integrin small molecule inhibitor at a dose of between about 20 mg to about 500 mg. In some embodiments, the method comprises administering the α4β7 integrin small molecule inhibitor at a dose of about 25 mg. In some embodiments, the method comprises administering the α4β7 integrin small molecule inhibitor at a dose of about 75 mg. In some embodiments, the method comprises administering the α4β7 integrin small molecule inhibitor at a dose of about 100 mg. In some embodiments, the method comprises administering the α4β7 integrin small molecule inhibitor at a dose of about 200 mg. In some embodiments, the α4β7 integrin small molecule inhibitor is administered by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, the α4β7 integrin small molecule inhibitor is administered orally. In some embodiments, the α4β7 small molecule inhibitor is dosed daily or every 2, 3, 4, 5, 6, 7, 8, 9, 10, 11, 12, 13, 14 or more days. In some embodiments, the α4β7 small molecule inhibitor is dosed 1, 2, 3, 4, 5, 6, 7, 8, 9, 10, 11, 12, 13, 14 or more times a day. In some embodiments, the α4β7 small molecule inhibitor is dosed 1 or more times a day every 1, 2, 3, 4, 5, 6, 7, 8, 9, 10, 11, 12, 13, 14 or more days. In some embodiments, the α4β7 small molecule inhibitor is dosed once daily. In some embodiments, the α4β7 integrin small molecule inhibitor is provided in a solid dosage form. In some embodiments, the α4β7 integrin small molecule inhibitor is administered under fasting conditions. In some embodiments, the fasting conditions means the subject does not consume food within 4 hours before administration of the α4β7 integrin small molecule inhibitor and within 2 hours after administration of the α4β7 integrin small molecule inhibitor. In some embodiments, the subject does not consume iron or calcium within 3 hours after administration of the α4β7 integrin small molecule inhibitor. In some embodiments, the method comprises administering a first dose (an induction dose) of the anti-IL-12/IL-23 antibody from between about 100 mg to about 2000 mg, between about 100 mg to about 1800 mg, between about 100 mg to about 1600 mg, between about 100 mg to about 1500 mg, between about 100 mg to about 1400 mg, between about 100 mg to about 1200 mg, between about 100 mg to about 1000 mg, between about 100 mg to about 800 mg, between about 100 mg to about 700 mg, between about 100 mg to about 600 mg, between about 150 mg to about 2000 mg, between about 150 mg to about 1800 mg, between about 150 mg to about 1600 mg, between about 150 mg to about 1500 mg, between about 150 mg to about 1400 mg, between about 150 mg to about 1200 mg, between about 150 mg to about 1000 mg, between about 150 mg to about 800 mg, between about 150 mg to about 700 mg, between about 150 mg to about 600 mg, between about 200 mg to about 2000 mg, between about 200 mg to about 1800 mg, between about 200 mg to about 1600 mg, between about 200 mg to about 1500 mg, between about 200 mg to about 1400 mg, between about 200 mg to about 1200 mg, between about 200 mg to about 1000 mg, between about 200 mg to about 800 mg, between about 200 mg to about 700 mg, between about 200 mg to about 600 mg. In some embodiments, the method comprises administering a first dose (an induction dose) from between about 200 mg to about 600 mg of the anti-IL-12/IL-23 antibody. In some embodiments, the method comprises administering a first dose (an induction dose) from between about 260 mg to about 520 mg of the anti-IL-12/IL-23 antibody. In some embodiments, the induction dose is about 260 mg of the anti-IL-12/IL-23 antibody. In some embodiments, the induction dose is about 390 mg of the anti-IL-12/IL-23 antibody. In some embodiments, the induction dose is about 520 mg of the anti-IL-12/IL-23 antibody. In some embodiments, the induction dose is determined by body weight (kg) of the subject. In some embodiments, the induction dose is (A) about 260 mg of the anti-IL-12/IL-23 antibody for subjects weighing less than or equal to 55 kg; (B) about 390 mg of the anti-IL-12/IL-23 antibody for subjects weighing greater than 55 kg, but less than or equal to 85 kg; or (C) about 520 mg of the anti-IL-12/IL-23 antibody for subjects weighing greater than 85 kg. In some embodiments, the induction dose is about 6.0 mg of the anti-IL-12/IL-23 antibody per kg of body weight. In some embodiments, the induction dose of the anti-IL-12/IL-23 antibody is administered on the same day as the first dose of the α4β7 integrin small molecule inhibitor. In some embodiments, the administration of the induction dose of the anti-IL-12/IL-23 antibody is by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, the administration of the induction dose of the anti-IL-12/IL-23 antibody is by intravenous (IV) injection. In some embodiments, the method further comprises administering 1, 2, 3, 4, 5, 6, 7, 8, 9, 10 or more maintenance doses of the anti-IL-12/IL-23 antibody. In some embodiments, at least one maintenance dose is between about 10 mg to about 200 mg of the anti-IL-12/IL-23 antibody. In some embodiments, at least one maintenance dose is about 45 mg of the anti-IL-12/IL-23 antibody. In some embodiments, at least one maintenance dose is about 90 mg of the anti-IL-12/IL-23 antibody. In some embodiments, at least one maintenance dose is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks after the induction dose. In some embodiments, at least one maintenance dose is administered at least 8, 16, 24, 32, 40, or 48 weeks after the induction dose. In some embodiments, at least 2 maintenance doses are administered at least 1, 2, 3, 4, 5, 6, 7, 8, 9, 10, 11, 12, 13, 14, 15 or more weeks apart. In some embodiments, at least 2 maintenance doses are administered at least 8 or more weeks apart. In some embodiments, at least 2 maintenance doses are administered at least 12 or more weeks apart. In some embodiments, at least 2 maintenance doses are administered every 1, 2, 3, 4, 5, 6, 7, 8, 9, 10, 11, 12, 13, 14, 15 or more weeks. In some embodiments, at least 2 maintenance doses are administered at least every 8 weeks. In some embodiments, at least 2 maintenance doses are administered at least every 12 weeks. In some embodiments, the method further comprises administering 1, 2, 3, 4, 5, 6 or more maintenance doses of about 90 mg of the anti-IL-12/IL-23 antibody at 8, 16, 24, 32, 40, and 48 weeks after the induction dose. In some embodiments, the method comprises treating the subject for at least 12 weeks, at least 24 weeks, at least 32 weeks, at least 40 weeks, at least 48 weeks, at least 52 weeks, at least 76 weeks, or longer. In some embodiments, the administration of the maintenance dose of the anti-IL-12/IL-23 antibody is by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, at least one maintenance dose of the anti-IL-12/IL-23 antibody is administered by subcutaneous (SC) injection. In some embodiments, at least one maintenance dose is administered on the same day as one or more doses of the α4β7 integrin small molecule inhibitor. In some embodiments, the inflammatory bowel disease is ulcerative colitis (UC). In some embodiments, the UC is moderately to severely active UC. In some embodiments, the subject has a received prior treatment of one or more conventional or advanced therapies for UC. Conventional therapies used for treatment of moderately to severely active UC include, for example oral corticosteroids, and immunomodulators such as azathioprine and 6 mercaptopurine (6-MP). Advanced therapies presently employed for treating moderately to severely active UC include biologic agents, such as for example, tumor necrosis factor alpha (TNFα) antagonists (i.e., as infliximab (Remicade®), adalimumab (Humira®), and golimumab (Simponi®)), monoclonal antibodies against interleukin (IL)-12 and IL-23 (i.e., ustekinumab (Stelara®))) inhibitors, and monoclonal antibodies against α4β7 integrin, (i.e., vedolizumab (Entyvio®)). Advance therapies for treatment of moderately to severely active UC also include small molecule such as for example, Janus kinase (JAK) antagonists (i.e., upadacitinib (Rinvoq®), tofacitinib (Xeljanz®), and filgotinib (Jyseleca®)), and a sphingosine-1-phosphate receptor modulator (i.e., ozanimod (Zeposia®)). In some embodiments, the inflammatory bowel disease is Crohn's disease. In some embodiments, treating the IBD results in corticosteroid-free clinical remission of the IBD. In some embodiments, treating the IBD results in clinical remission of the IBD. In some embodiments, treating the IBD results in mucosal healing. In some embodiments, the subject is a human. In some embodiments, the subject does not suffer from chronic or active infections. In some embodiments, the subject does not have a predisposition to infections.

Disclosed herein is a method for treating and/or preventing inflammatory bowel disease (IBD) in a subject, comprising co-administering to the subject (A) a therapeutically effective amount of an α4β7 integrin small molecule inhibitor; and (B) a therapeutically effective amount of an anti-IL-12/IL-23 antibody or an antigen-binding fragment thereof. In some embodiments, the α4β7 integrin small molecule inhibitor is a compound of Formula (I):

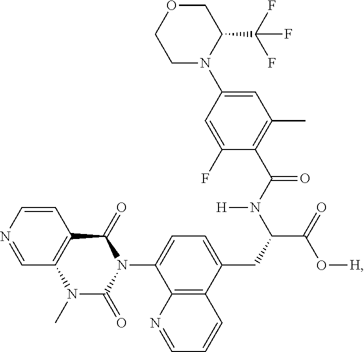

or a pharmaceutically acceptable salt thereof. In some embodiments, the α4β7 integrin small molecule inhibitor is a compound of Formula (II):

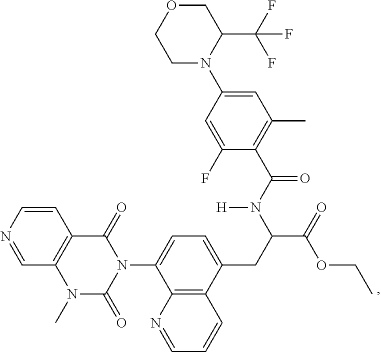 [SMILES: CCOC(=O)C(Cc1ccc(-n2c(=O)c3ccncc3n(C)c2=O)c2ncccc12)NC(=O)c1c(C)cc(N2CCOCC2C(F)(F)F)cc1F]

or a pharmaceutically acceptable salt thereof. In some embodiments, the α4β7 integrin small molecule inhibitor is a compound of Formula (III):

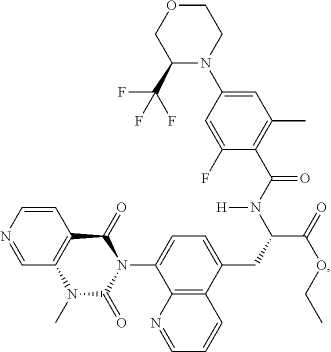

or a pharmaceutically acceptable salt thereof. In some embodiments, the α4β7 integrin small molecule inhibitor is a compound of Formula (IV):

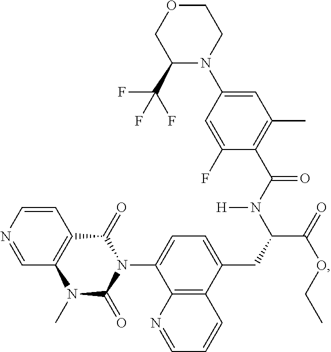

or a pharmaceutically acceptable salt thereof. In some embodiments, the anti-IL-12/IL-23 antibody is selected from risankizumab, ustekinumab, guselkumab, brazikumab, mirikizumab, tidarakizumab, briakinumab, and an antigen binding fragment thereof. In some embodiments, the anti-IL-12/IL-23 antibody is ustekinumab or an antigen binding fragment thereof. In some embodiments, the anti-IL-12/IL-23 antibody comprises (A) a heavy chain comprising (I) a heavy chain complementarity-determining region (HCDR) 1 comprising SEQ ID NO: 1; (II) a HCDR2 comprising SEQ ID NO: 2; and (III) a HCDR3 comprising SEQ ID NO: 3; and (B) a light chain comprising (I) a light chain complementarity-determining region (LCDR) 2 comprising SEQ ID NO: 4; (II) a LCDR2 comprising SEQ ID NO: 5; and (III) a LCDR3 comprising SEQ ID NO: 6. In some embodiments, the heavy chain of the anti-IL-12/IL-23 antibody comprises a heavy chain variable region (VH) sequence comprising SEQ ID NO: 7. In some embodiments, the heavy chain of the anti-IL-12/IL-23 antibody comprises a VH sequence that is at least about 90%, 91%, 92%, 93%, 94%, 95%, 96%, 97%, 98%, 99% or 100% identical to the amino acid sequence of SEQ ID NO: 7. In some embodiments, the heavy chain of the anti-IL-12/IL-23 antibody comprises a VH sequence that is at least about 95% identical to the amino acid sequence of SEQ ID NO: 7. In some embodiments, the heavy chain of the anti-IL-12/IL-23 antibody comprises a VH sequence that is at least about 97% identical to the amino acid sequence of SEQ ID NO: 7. In some embodiments, the heavy chain of the anti-IL-12/IL-23 antibody comprises a VH sequence that is at least about 98% identical to the amino acid sequence of SEQ ID NO: 7. In some embodiments, the heavy chain of the anti-IL-12/IL-23 antibody comprises a VH sequence that is at least about 99% identical to the amino acid sequence of SEQ ID NO: 7. In some embodiments, the heavy chain of the anti-IL-12/IL-23 antibody comprises a VH sequence that is about 100% identical to the amino acid sequence of SEQ ID NO: 7. In some embodiments, the heavy chain of the anti-IL-12/IL-23 antibody comprises a full-length heavy chain sequence comprising SEQ ID NO: 9. In some embodiments, the heavy chain of the anti-IL-12/IL-23 antibody comprises a full-length heavy chain sequence that is at least about 90%, 91%, 92%, 93%, 94%, 95%, 96%, 97%, 98%, 99% or 100% identical to the amino acid sequence of SEQ ID NO: 9. In some embodiments, the heavy chain of the anti-IL-12/IL-23 antibody comprises a full-length heavy chain sequence that is at least about 95% identical to the amino acid sequence of SEQ ID NO: 9. In some embodiments, the heavy chain of the anti-IL-12/IL-23 antibody comprises a full-length heavy chain sequence that is at least about 97% identical to the amino acid sequence of SEQ ID NO: 9. In some embodiments, the heavy chain of the anti-IL-12/IL-23 antibody comprises a full-length heavy chain sequence that is at least about 98% identical to the amino acid sequence of SEQ ID NO: 9. In some embodiments, the heavy chain of the anti-IL-12/IL-23 antibody comprises a full-length heavy chain sequence that is at least about 99% identical to the amino acid sequence of SEQ ID NO: 9. In some embodiments, the heavy chain of the anti-IL-12/IL-23 antibody comprises a full-length heavy chain sequence that is 100% identical to the amino acid sequence of SEQ ID NO: 9. In some embodiments, the light chain of the anti-IL-12/IL-23 antibody comprises a light chain variable region (VL) sequence comprising SEQ ID NO: 8. In some embodiments, the light chain of the anti-IL-12/IL-23 antibody comprises a VL sequence that is at least about 90%, 91%, 92%, 93%, 94%, 95%, 96%, 97%, 98%, 99% or 100% identical to the amino acid sequence of SEQ ID NO: 8. In some embodiments, the light chain of the anti-IL-12/IL-23 antibody comprises a VL sequence that is at least about 95% identical to the amino acid sequence of SEQ ID NO: 8. In some embodiments, the light chain of the anti-IL-12/IL-23 antibody comprises a VL sequence that is at least about 97% identical to the amino acid sequence of SEQ ID NO: 8. In some embodiments, the light chain of the anti-IL-12/IL-23 antibody comprises a VL sequence that is at least about 98% identical to the amino acid sequence of SEQ ID NO: 8. In some embodiments, the light chain of the anti-IL-12/IL-23 antibody comprises a VL sequence that is at least about 99% identical to the amino acid sequence of SEQ ID NO: 8. In some embodiments, the light chain of the anti-IL-12/IL-23 antibody comprises a VL sequence that is 100% identical to the amino acid sequence of SEQ ID NO: 8. In some embodiments, the light chain of the anti-IL-12/IL-23 antibody comprises a full-length light chain sequence comprising SEQ ID NO: 10. In some embodiments, the light chain of the anti-IL-12/IL-23 antibody comprises a full-length light chain sequence that is at least about 90%, 91%, 92%, 93%, 94%, 95%, 96%, 97%, 98%, 99% or 100% identical to the amino acid sequence of SEQ ID NO: 10. In some embodiments, the light chain of the anti-IL-12/IL-23 antibody comprises a full-length light chain sequence that is at least about 95% identical to the amino acid sequence of SEQ ID NO: 10. In some embodiments, the light chain of the anti-IL-12/IL-23 antibody comprises a full-length light chain sequence that is at least about 97% identical to the amino acid sequence of SEQ ID NO: 10. In some embodiments, the light chain of the anti-IL-12/IL-23 antibody comprises a full-length light chain sequence that is at least about 98% identical to the amino acid sequence of SEQ ID NO: 10. In some embodiments, the light chain of the anti-IL-12/IL-23 antibody comprises a full-length light chain sequence that is at least about 99% identical to the amino acid sequence of SEQ ID NO: 10. In some embodiments, the light chain of the anti-IL-12/IL-23 antibody comprises a full-length light chain sequence that is 100% identical to the amino acid sequence of SEQ ID NO: 10. In some embodiments, the anti-IL-12/IL-23 antibody is a chimeric, humanized, veneered, or human antibody. In some embodiments, the anti-IL-12/IL-23 antibody is a humanized antibody. In some embodiments, the anti-IL-12/IL-23 antibody comprises an immunoglobulin (Ig) heavy chain selected from gamma (G), alpha (A), delta (D), epsilon (E), or mu (M). In some embodiments, the Ig heavy chain comprises an immunoglobulin gamma (IgG) heavy chain. In some embodiments, the IgG heavy chain is selected from IgG1, IgG2, IgG3, or IgG4. In some embodiments, the IgG heavy chain comprises IgG1. In some embodiments, the IgG heavy chain comprises an IgG1 heavy chain that has up to 10 amino acid substitutions in the constant region. In some embodiments, the IgG heavy chain comprises IgG4. In some embodiments, the IgG heavy chain comprises an IgG4 heavy chain that has up to 10 amino acid substitutions in the constant region. In some embodiments, the heavy chain of the anti-IL-12/IL-23 antibody comprises an Fc region that comprises one or more of the following amino acids: alanine (A) at position 234, alanine (A) at position 235, aspartic acid (D) at position 236, aspartic acid (D) at position 237, aspartic acid (D) at position 238, alanine (A) at position 265, glutamic acid (E) at position 267, glycine (G) at position 271, arginine (R) at position 330, alanine (A) at position 332, or alanine (A) at position 297 (all numbering according to EU Index). In some embodiments, the anti-IL-12/IL-23 antibody comprises a human IgG1 antibody having one or more substitutions selected from G236D, G237D, P238D, D265A, S267E, P271G, A330R, K322A, and N297A (all numbering according to EU Index). In some embodiments, the anti-IL-12/IL-23 antibody comprises a human IgG4 antibody having one or more substitutions selected from P238D, G237D, P271G, S330R, F234A and L235A (all numbering according to EU Index). In some embodiments, the method comprises administering the α4β7 integrin small molecule inhibitor at a dose of between about 5 mg to about 2000 mg, between about 5 mg to about 1800 mg, between about 5 mg to about 1600 mg, between about 5 mg to about 1500 mg, between about 5 mg to about 1400 mg, between about 5 mg to about 1200 mg, between about 5 mg to about 1000 mg, between about 5 mg to about 800 mg, between about 5 mg to about 700 mg, between about 5 mg to about 600 mg, between about 5 mg to about 500 mg, between about 5 mg to about 400 mg, between about 5 mg to about 300 mg, between about 5 mg to about 250 mg, between about 5 mg to about 200 mg, between 10 mg to about 2000 mg, between about 10 mg to about 1800 mg, between about 10 mg to about 1600 mg, between about 10 mg to about 1500 mg, between about 10 mg to about 1400 mg, between about 10 mg to about 1200 mg, between about 10 mg to about 1000 mg, between about 10 mg to about 800 mg, between about 10 mg to about 700 mg, between about 10 mg to about 600 mg, between about 10 mg to about 500 mg, between about 10 mg to about 400 mg, between about 10 mg to about 300 mg, between about 10 mg to about 250 mg, between about 10 mg to about 200 mg, between about 15 mg to about 2000 mg, between about 15 mg to about 1800 mg, between about 15 mg to about 1600 mg, between about 15 mg to about 1500 mg, between about 15 mg to about 1400 mg, between about 15 mg to about 1200 mg, between about 15 mg to about 1000 mg, between about 15 mg to about 800 mg, between about 15 mg to about 700 mg, between about 5 mg to about 600 mg, between about 15 mg to about 500 mg, between about 15 mg to about 400 mg, between about 15 mg to about 300 mg, between about 15 mg to about 250 mg, between about 15 mg to about 200 mg, between about 20 mg to about 2000 mg, between about 20 mg to about 1800 mg, between about 20 mg to about 1600 mg, between about 20 mg to about 1500 mg, between about 20 mg to about 1400 mg, between about 20 mg to about 1200 mg, between about 20 mg to about 1000 mg, between about 20 mg to about 800 mg, between about 20 mg to about 700 mg, between about 20 mg to about 600 mg, between about 20 mg to about 500 mg, between about 20 mg to about 400 mg, between about 20 mg to about 300 mg, between about 20 mg to about 250 mg, or between about 20 mg to about 200 mg. In some embodiments, the method comprises administering the α4β7 integrin small molecule inhibitor at a dose of between about 20 mg to about 1000 mg. In some embodiments, the method comprises administering the α4β7 integrin small molecule inhibitor at a dose of between about 20 mg to about 500 mg. In some embodiments, the method comprises administering the α4β7 integrin small molecule inhibitor at a dose of about 25 mg. In some embodiments, the method comprises administering the α4β7 integrin small molecule inhibitor at a dose of about 75 mg. In some embodiments, the method comprises administering the α4β7 integrin small molecule inhibitor at a dose of about 100 mg. In some embodiments, the method comprises administering the α4β7 integrin small molecule inhibitor at a dose of about 200 mg. In some embodiments, the α4β7 integrin small molecule inhibitor is administered by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, the α4β7 integrin small molecule inhibitor is administered orally. In some embodiments, the α4β7 small molecule inhibitor is dosed daily or every 2, 3, 4, 5, 6, 7, 8, 9, 10, 11, 12, 13, 14 or more days. In some embodiments, the α4β7 small molecule inhibitor is dosed 1, 2, 3, 4, 5, 6, 7, 8, 9, 10, 11, 12, 13, 14 or more times a day. In some embodiments, the α4β7 small molecule inhibitor is dosed 1 or more times a day every 1, 2, 3, 4, 5, 6, 7, 8, 9, 10, 11, 12, 13, 14 or more days. In some embodiments, the α4β7 small molecule inhibitor is dosed once daily. In some embodiments, the α4β7 integrin small molecule inhibitor is provided in a solid dosage form. In some embodiments, the α4β7 integrin small molecule inhibitor is administered under fasting conditions. In some embodiments, the fasting conditions means the subject does not consume food within 4 hours before administration of the α4β7 integrin small molecule inhibitor and within 2 hours after administration of the α4β7 integrin small molecule inhibitor. In some embodiments, the subject does not consume iron or calcium within 3 hours after administration of the α4β7 integrin small molecule inhibitor. In some embodiments, the method comprises administering a first dose (an induction dose) of the anti-IL-12/IL-23 antibody from between about 100 mg to about 2000 mg, between about 100 mg to about 1800 mg, between about 100 mg to about 1600 mg, between about 100 mg to about 1500 mg, between about 100 mg to about 1400 mg, between about 100 mg to about 1200 mg, between about 100 mg to about 1000 mg, between about 100 mg to about 800 mg, between about 100 mg to about 700 mg, between about 100 mg to about 600 mg, between about 150 mg to about 2000 mg, between about 150 mg to about 1800 mg, between about 150 mg to about 1600 mg, between about 150 mg to about 1500 mg, between about 150 mg to about 1400 mg, between about 150 mg to about 1200 mg, between about 150 mg to about 1000 mg, between about 150 mg to about 800 mg, between about 150 mg to about 700 mg, between about 150 mg to about 600 mg, between about 200 mg to about 2000 mg, between about 200 mg to about 1800 mg, between about 200 mg to about 1600 mg, between about 200 mg to about 1500 mg, between about 200 mg to about 1400 mg, between about 200 mg to about 1200 mg, between about 200 mg to about 1000 mg, between about 200 mg to about 800 mg, between about 200 mg to about 700 mg, between about 200 mg to about 600 mg. In some embodiments, the method comprises administering a first dose (an induction dose) from between about 200 mg to about 600 mg of the anti-IL-12/IL-23 antibody. In some embodiments, the method comprises administering a first dose (an induction dose) from between about 260 mg to about 520 mg of the anti-IL-12/IL-23 antibody. In some embodiments, the induction dose is about 260 mg of the anti-IL-12/IL-23 antibody. In some embodiments, the induction dose is about 390 mg of the anti-IL-12/IL-23 antibody. In some embodiments, the induction dose is about 520 mg of the anti-IL-12/IL-23 antibody. In some embodiments, the induction dose is determined by body weight (kg) of the subject. In some embodiments, the induction dose is (A) about 260 mg of the anti-IL-12/IL-23 antibody for subjects weighing less than or equal to 55 kg; (B) about 390 mg of the anti-IL-12/IL-23 antibody for subjects weighing greater than 55 kg, but less than or equal to 85 kg; or (C) about 520 mg of the anti-IL-12/IL-23 antibody for subjects weighing greater than 85 kg. In some embodiments, the induction dose is about 6.0 mg of the anti-IL-12/IL-23 antibody per kg of body weight. In some embodiments, the induction dose of the anti-IL-12/IL-23 antibody is administered on the same day as the first dose of the α4β7 integrin small molecule inhibitor. In some embodiments, the administration of the induction dose of the anti-IL-12/IL-23 antibody is by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, the administration of the induction dose of the anti-IL-12/IL-23 antibody is by intravenous (IV) injection. In some embodiments, the method further comprises administering 1, 2, 3, 4, 5, 6, 7, 8, 9, 10 or more maintenance doses of the anti-IL-12/IL-23 antibody. In some embodiments, at least one maintenance dose is between about 10 mg to about 200 mg of the anti-IL-12/IL-23 antibody. In some embodiments, at least one maintenance dose is about 45 mg of the anti-IL-12/IL-23 antibody. In some embodiments, at least one maintenance dose is about 90 mg of the anti-IL-12/IL-23 antibody. In some embodiments, at least one maintenance dose is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks after the induction dose. In some embodiments, at least one maintenance dose is administered at least 8, 16, 24, 32, 40, or 48 weeks after the induction dose. In some embodiments, at least 2 maintenance doses are administered at least 1, 2, 3, 4, 5, 6, 7, 8, 9, 10, 11, 12, 13, 14, 15 or more weeks apart. In some embodiments, at least 2 maintenance doses are administered at least 8 or more weeks apart. In some embodiments, at least 2 maintenance doses are administered at least 12 or more weeks apart. In some embodiments, at least 2 maintenance doses are administered every 1, 2, 3, 4, 5, 6, 7, 8, 9, 10, 11, 12, 13, 14, 15 or more weeks. In some embodiments, at least 2 maintenance doses are administered at least every 8 weeks. In some embodiments, at least 2 maintenance doses are administered at least every 12 weeks. In some embodiments, the method further comprises administering 1, 2, 3, 4, 5, 6 or more maintenance doses of about 90 mg of the anti-IL-12/IL-23 antibody at 8, 16, 24, 32, 40, and 48 weeks after the induction dose. In some embodiments, the method comprises treating the subject for at least 12 weeks, at least 24 weeks, at least 32 weeks, at least 40 weeks, at least 48 weeks, at least 52 weeks, at least 76 weeks or longer. In some embodiments, the administration of the maintenance dose of the anti-IL-12/IL-23 antibody is by one or more routes of administration selected from oral, parenteral, topical, or by inhalation. In some embodiments, parenteral administration comprises intravenous, intraarterial, intraperitoneal, intramuscular, subcutaneous, rectal, or vaginal administration. In some embodiments, at least one maintenance dose of the anti-IL-12/IL-23 antibody is administered by subcutaneous (SC) injection. In some embodiments, at least one maintenance dose is administered on the same day as one or more doses of the α4β7 integrin small molecule inhibitor. In some embodiments, the inflammatory bowel disease is ulcerative colitis (UC). In some embodiments, the UC is moderately to severely active UC. In some embodiments, the subject has a received prior treatment of one or more conventional or advanced therapies for UC. In some embodiments, the inflammatory bowel disease is Crohn's disease. In some embodiments, treating the IBD results in corticosteroid-free clinical remission of the IBD. In some embodiments, treating the IBD results in clinical remission of the IBD. In some embodiments, treating the IBD results in mucosal healing. In some embodiments, the subject is a human. In some embodiments, the subject does not suffer from chronic or active infections. In some embodiments, the subject does not have a predisposition to infections.

### EXAMPLES

As used herein in the examples Formula (III) is a compound of Formula (III)

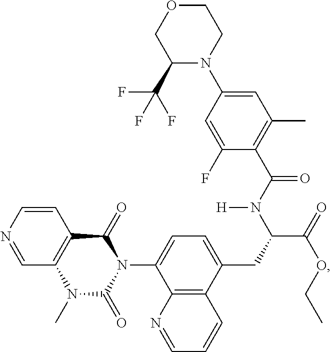

As used in the Examples Section herein term “metabolite” refers to a compound of Formula (I)

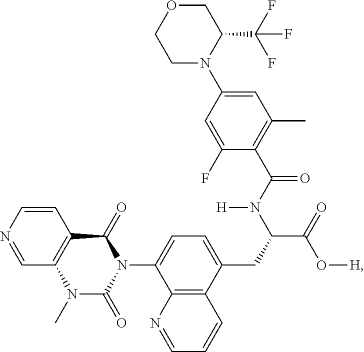

Methods for making compounds of Formula's (I) and (III) can be found in Granted U.S. Pat. No. 11,116,760, which are hereby incorporated by reference.

### Example 1. Study of α4β7 Integrin Small Molecule Inhibitor to Evaluate Pharmacokinetics, Pharmacodynamics, Safety, and Tolerability in Healthy Volunteers

This was a 3-part study to evaluate pharmacokinetics, safety, tolerability, and pharmacodynamics of the Formula (III) compound in healthy volunteers.
To understand the single-dose PK, safety, tolerability, and PD of Formula (III) and its active metabolite, subjects in Part A received a single dose of Formula (III) in the fasting state in a dose-escalating manner. Food intake may affect the rate and extent of absorption of Formula (III), therefore the PK of a dose of 200 mg Formula (III) following a standard meal was characterized.
To understand the multiple-dose PK, safety, tolerability, and PD of Formula (III) and its active metabolite, subjects in Part B received multiple doses of Formula (III) were administered once daily in a dose-escalating manner.
To understand the relative BA of a tablet formulation compared to the capsule formulation and to understand the effect of concomitant food intake or a representative acid-reducing agent (ARA) (omeprazole) on the PK of Formula (III) (tablet) and its active metabolite, subjects in Part C received Formula (III) along with and without a high-fat/high-calorie meal and an ARA.

This study entails administration of Formula (III) compound in humans for the first time. The objectives of this first-in-human (FIH) study were to evaluate the PK, safety, tolerability, and PD of Formula (III) compound after single-ascending doses (SAD) and multiple-ascending doses (MAD) of Formula (III) (Parts A and B, respectively). The study also included a Part C. The objectives of Part C were to evaluate the relative bioavailability (BA) of a tablet formulation versus a capsule formulation, to evaluate the effect of concomitant food intake on the PK of Formula (III) and its active metabolite, and to evaluate the effect of a representative ARA (omeprazole) on the PK of Formula (III) and its active metabolite.

The results from this study formed the basis for further evaluation of Formula (III) compound and dose selection for Experiment 4 in subjects with IBD.

### Participant Population

### Number of Participants Enrolled:

A total of 148 unique subjects were enrolled in the study: 50 participants in Part A, 62 participants in Part B, and 36 participants in Part C (18 each in Cohorts 11 and 12).

Subjects were admitted into the clinic at Day −2 in Parts A and B in order to assess the impact of circadian rhythm of circulating biomarkers prior to Formula (IIII) dosing.

Part A consisted of up to 5 randomized, blinded (sponsor-unblinded), placebo-controlled, single-dose escalation cohorts. Within each cohort, 10 unique subjects were randomized 4:1 to receive blinded Formula (III) (N=8) or placebo-to-match (PTM) Formula (III) (N=2).

Part A (SAD Cohorts 1, 2, 4, and 5) consisted of SAD cohorts.

Part A (SAD Cohort 3) consisted of a single dose of Formula (III) on Day 1 in addition to a single dose of Formula (III) on Day 8. Those who received Formula (III) on Day 1 also received Formula (III) Formula (III) on Day 8, whereas those randomized to PTM Formula (III) on Day 1 received PTM Formula (III) on Day 8.

Part B (MAD Cohorts 6-10) consisted of up to 5 randomized, blinded (Sponsor-unblinded), placebo-controlled, MAD cohorts. Within each cohort 12 unique subjects were randomized 3:1 to receive blinded Formula (III) (N=9) or PTM Formula (III) (N=3). All subjects within each cohort received Formula (III) at the same dosing frequency.

Part C (Relative BA/Food effect/ARA effect, Cohort 11 and Cohort 12) were open-label, randomized, 3-way crossover cohorts with 4 (Cohort 11) or 3 (Cohort 12) treatment periods assessing single doses of Formula (III) in capsule and tablet formulation. Based on safety, tolerability, and any relevant PK and PD data from Part A, the doses of Formula (III) for Cohort 11 and Cohort 12 were chosen at or below the half of the highest dose that was found to be safe and well tolerated in Part A, and the dose for Cohort 12 were different from Cohort 11 to allow evaluation of the relative BA and food effect at 2 clinically relevant doses. Following completion of screening and admission (Day −1) procedures, eligible subjects were enrolled and randomized 1:1:1 into 3 treatment sequence groups to receive study treatments as outlined in Table 3 for Cohort 11 and Table 4 for Cohort 12.

TABLE 3
Treatment Sequences for Part C Cohort 11 (Relative BA/Food Effect/ARA Effect)

| | Study Days 1-7 | | Study Days 8-14 | | Study Days 15-18 | | Study Days 19-28 | | |
|---|---|---|---|---|---|---|---|---|---|
| Sequence | Period 1 | | Period 2 | | Period 3 | | Period 4 | | |
| Group | Day 1 | Days 2-7 | Day 1 | Day 2-7 | Day 1 | Days 2-4 | Days 1-5 | Day 6 | Days 7-10 |

| | | | | | | | | |
|---|---|---|---|---|---|---|---|---|
| 1 | C | Washout | D | Washout | Washout | F | G | No |
| | | | | | | | | treatment |
| 2 | D | Washout | E | Washout | Washout | F | G | No |
| | | | | | | | | treatment |
| 3 | E | Washout | C | Washout | Washout | F | G | No |
| | | | | | | | | treatment |

The Study treatments are as follows:
Treatment C: 200 mg single dose of Formula (III) as capsule formulation, fasting.
Treatment D: 200 mg single dose of Formula (III) as tablet formulation, fasting.
Treatment E: 200 mg single dose of Formula (III) as tablet formulation, nonfasting (high-fat/high-calorie meal).
Treatment F: 40 mg omeprazole, fasting.
Treatment G: 40 mg omeprazole followed 2 hours later by 200 mg single dose of Formula (III) as tablet formulation, fasting.

TABLE 4
Treatment Sequences for Part C Cohort 12 (Relative BA/Food Effect)

| Sequence | Study Days 1-7 Period 1 | | Study Days 8-14 Period 2 | | Study Days 15-20 Period 3 | |
|---|---|---|---|---|---|---|
| Group | Day 1 | Days 2-7 | Day 1 | Days 2-7 | Day 1 | Days 2-6 |
| 1 | H | Washout | I | Washout | J | No |
| | | | | | | treatment |
| 2 | I | Washout | J | Washout | H | No |
| | | | | | | treatment |
| 3 | J | Washout | H | Washout | I | No |
| | | | | | | treatment |

The study treatments are as follows:
Treatment H: 20 mg single dose of Formula (III) capsule formulation fasting, different does level compared to Treatment C.
Treatment I: 20 mg single dose of Formula (III) as tablet formulation, fasting.
Treatment J: 20 mg single dose of Formula (III) as a tablet formulation, nonfasting, (high-fat/High/calorie meal).

Part B (Cohorts 6-10) and C Cohorts 11-12 may run in parallel.

### Study Population:

### Inclusion Criteria:

Subjects had to meet all of the following inclusion criteria to be eligible for participation in this study:
1. Have the ability to understand and sigh a written informed consent from (ICF), which must be obtained prior to initiation of study procedures.
2. Be aged 18 through 55 years, inclusive, at screening.
3. Be a nonsmoker. The use of nicotine or nicotine-containing products must be discontinued 90 days prior to the first dose of study drug.
4. Have a calculated body mass index (BMI) of ≥19 and ≤30 kg/m² at screening.
5. Have a creatinine clearance (CL_{cr})≥90 mL/min (using the Cockcroft-Gault method (Cockcroft D W, Gault M H. Prediction of creatinine clearance from serum creatinine. Nephron 1976; 16:31-41) based on serum creatinine and actual body weight as measured at screening and upon admission:

$$a . Male \text{:} \underline{( 140 - Age [ years ] \times ( Weight [ kg ] )} = {CL}_{cr} ( mL / min ) 72 \times ( Serum Creatinine [ mg / dL ] )$$

$$b . Female \text{:} \underline{( 140 - Age [ years ] \times ( Weight [ kg ] )} \times 0.85 = {CL}_{cr} ( mL / min ) 72 \times ( Serum Creatinine [ mg / dL ] )$$

6. Male subjects and female subjects of childbearing potential who engage in heterosexual intercourse must agree to use contraception.
7. Subjects must refrain from blood donation from clinic admission, throughout the study period, and continuing for at least 56 days following the last dose of study drug.
8. Screening laboratory evaluations and 12-lead electrocardiogram (ECG) evaluations must be without clinically significant abnormalities as assessed by the investigator.
9. Have liver biometric tests such as alanine aminotransferase (ALT), aspartate aminotransferase (AST), alkaline phosphatase, and total bilirubin below the upper limit of normal at screening.
10. No evidence of active or latent TB, which should be supported by a negative interferon gamma release assay (IGRA) test performed by the laboratory at screening.
11. Must be willing and able to comply with all study requirements.
12. Must, in the opinion of the investigator, be in good health based upon medical history and physical examination, including vital signs.

### Exclusion Criteria:

Subjects who meet any of the following exclusion criteria were not enrolled in this study:
1. Positive serum pregnancy test.
2. Breastfeeding female.
3. Female subjects who plan egg donation or in vitro fertilization from clinic admission (Day −2 in Parts A and B, Day −1 in Part C), throughout the study period, and/or within 14 days (or by the institutional guideline as indicated, whichever is longer) following the last dose of study drug.
4. Male subjects who plan to donate sperm from clinic admission (Day −2 in Parts A and B, Day −1 in Part C), throughout the study period, and/or within 14 days (or by the institutional guideline as indicated, whichever is longer) following the last dose of study drug.
5. Have received any investigational study drug within 30 days prior to study dosing.
6. Have current alcohol or substance abuse judged by the investigator to potentially interfere with subject compliance or subject safety, or a positive drug or alcohol test at screening or admission.
7. Have a positive test result for HIV-1 antibody, hepatitis B surface antigen (HBsAg), or hepatitis C virus (HCV) antibody at screening.
8. Have poor venous access that limits phlebotomy.
9. Have taken any prescription medications, or over-the-counter medications, including herbal products, within 28 days prior to start of study drug dosing, with the exception of vitamins and/or acetaminophen and/or ibuprofen and/or hormonal contraceptive medications.
10. Have been treated with systemic steroids, immunosuppressant therapies, or chemotherapeutic agents within 3 months prior to screening or is expected to receive these agents during the study (eg, corticosteroids, immunoglobulins, other immune- or cytokine-based therapies).
11. Have a history of any of the following:
   a. Significant serious skin disease, such as but not limited to rash, food allergy, eczema, psoriasis, or urticaria.
   b. Significant drug sensitivity or drug allergy (such as anaphylaxis or hepatoxicity).
   c. Known hypersensitivity to the study drugs, their metabolites or to formulation excipients.
   d. Significant cardiac disease (including history of myocardial infarction based on ECG and/or clinical history, any history of ventricular tachycardia, congestive heart failure, or dilated cardiomyopathy with left ventricular ejection fraction <40%), a family history of long QT syndrome, or unexplained death in an otherwise healthy immediate family member between the ages of 1 and 30 years.
   e. Recurrent palpitations, unexplained syncope or dizziness.
   f. Implanted defibrillator or pacemaker.
   g. Liver disease, including Gilbert syndrome.
   h. Severe peptic ulcer disease, gastroesophageal reflux disease, or other gastric acid hypersecretory conditions requiring prolonged (>6 months) medical treatment.
   i. Medical or surgical treatment that permanently altered gastric absorption (eg, gastric bypass or bariatric surgery). A history of cholecystectomy is not exclusionary.
12. Have any serious or active medical or psychiatric illness (including depression) that, in the opinion of the investigator, would interfere with subject treatment, assessment, or compliance with the protocol. This would include renal, cardiac, hematological, hepatic, pulmonary (including chronic asthma), endocrine (including diabetes), central nervous, gastrointestinal (including an ulcer), vascular, metabolic (thyroid disorders, adrenal disease), immunodeficiency disorders, active infection, or malignancy that are clinically significant or requiring treatment.
13. Subjects must not have donated blood within 56 days of study entry or plasma within 7 days of study entry.
14. Have received any vaccination within 14 days prior to the first dose of study drug.
15. Subjects with abnormal findings on neurological examination at screening or Day −2 in Parts A and B, Day −1 in Part C, prior to dosing.

### Part C (Cohorts 11 and 12) was an Open-Label, Unblinded Evaluation.

### Dosage and Administration of Formula (III)

### Part A (SAD): Cohorts 1-5 (Table 5)

### Cohorts 1, 2, 4, and 5

Subjects enrolled in Cohorts 1, 2, 4, and 5 received either blinded Formula (III) or PTM Formula (III) as a single dose on Day 1. All study drugs were administered in the morning in a fasted state.

### Cohort 3

Subjects enrolled in Cohort 3 received a single dose of Formula (III) (up to 200 mg) or PTM Formula (III) under fasting conditions on Day 1 (Treatment A). On Day 8, subjects received the same single dose of Formula (III) (up to 200 mg) or PTM Formula (III) within 5 minutes of completing a standard meal (Treatment B).

TABLE 5
The cohorts and study treatments for Part A

| Cohort | Single-Ascending Dose | Day(s) |
|---|---|---|
| 1 | 20 mg Formula (III) or PTM Formula (III), | 1 |
| | single-dose, fasting, capsule formulation | |
| 2 | 60 mg Formula (III) or PTM Formula (III), | 1 |
| | single-dose, fasting, capsule formulation | |
| 3 | 200 mg Formula (III) or PTM Formula (III), | 1 and 8 |
| | single-dose, under fasting conditions on Day 1 | |
| | (Treatment A) and nonfasting conditions on Day 8 | |
| | (Treatment B), capsule formulation | |
| 4 | 500 mg Formula (III) or PTM Formula (III), | 1 |
| | single-dose, fasting, capsule formulation | |
| 5 | 1000 mg Formula (III) or PTM Formula (III), | 1 |
| | single-dose, fasting, capsule formulation | |

PTM = placebo-to-match

### Part B (MAD): Cohorts 6-10 (Table 6)

Subjects enrolled in Part B received either blinded Formula (III) or PTM Formula (III) twice daily or once daily for 14 days. All subjects within each cohort received study drug at the same dosing frequency.

All study drugs in Part B (except Cohort 10 which will be administered under fasting conditions) were administered in the morning and evening following a standard meal (twice daily dosing), or only in the morning (daily dosing) following a standard meal except for Cohort 10.

TABLE 6
The cohorts and study treatments for Part B

| Cohort | Multiple-Ascending Dose (Days 1-14) |
|---|---|
| 6 | 20 mg Formula (III) or PTM Formula (III), QD, |
| | nonfasting, capsule formulation or |
| 7 | 80 mg Formula (III) or PTM Formula (III), QD, |
| | nonfasting, capsule formulation or |
| 8 | 200 mg Formula (III) or PTM Formula (III), QD, |
| | nonfasting, capsule formulation or |
| 9 | 500 mg Formula (III) or PTM Formula (III), QD, |
| | nonfasting, capsule formulation or |
| 10 | 200 mg Formula (III) or PTM Formula (III), QD |
| | fasting, capsule or tablet formulation or |

BID = twice daily;
PTM = placebo-to-match;
QD = once daily

For twice daily dosing, both morning and evening doses needed to be administered on Day 14.

### Part C (Relative BA/Food Effect/ARA Effect): Cohorts 11 and 12

Subjects enrolled in Part C received a single dose Formula (III) either in a capsule formulation or a tablet formulation. All study drugs in Part C will be administered in the morning under fasting state or following a high-fat/high-calorie breakfast.

The doses of Formula (III) for Cohorts 11 and 12 were chosen at or below half of the highest dose that was found to be safe and well tolerated in Part A. The dose for Cohort 12, if it proceeds, were chosen at a different dose compared to Cohort 11. The decision to conduct Cohort 12 were based on data from Cohort 11 as well as SAD and MAD cohorts. Following completion of screening and admission (Day −1) procedures, eligible subjects were enrolled and randomized 1:1:1 into 3 treatment sequence groups to receive study treatments as outlined in Table 7 (Cohort 11) or Table 8 (Cohort 12).

TABLE 7
Treatment Sequences for Part C Cohort 11 (Relative BA/Food Effect/ARA Effect).

| | Study Days 1-7 | | Study Days 8-14 | | Study Days 15-18 | | Study Days 19-28 | | |
|---|---|---|---|---|---|---|---|---|---|
| Sequence | Period 1 | | Period 2 | | Period 3 | | Period 4 | | |
| Group | Day 1 | Days 2-7 | Day 1 | Days 2-7 | Day 1 | Days 2-4 | Days 1-5 | Day 6 | Days 7-10 |
| 1 | C | washout | D | washout | E | washout | F | G | no treatment |
| 2 | D | washout | E | washout | C | washout | F | G | no treatment |
| 3 | E | washout | C | washout | D | washout | F | G | no treatment |

The study treatments are as follows:
Treatment C: 200 mg single dose of Formula (III) as capsule formulation, fasting.
Treatment D: 200 mg single dose of Formula (III) as tablet formulation, fasting.
Treatment E: 200 mg single dose of Formula (III) as tablet formulation, nonfasting (high-fat/high-calorie meal).
Treatment F: 40 mg omeprazole, fasting.
Treatment G: 40 mg omeprazole followed 2 hours later, by 200 mg single dose of Formula (III) as tablet formulation, fasting.

TABLE 8
Treatment Sequences for Part C Cohort 12 (Relative BA/Food Effect)

| | Study Days 1-7 | | Study Days 8-14 | | Study Days 15-20 | |
|---|---|---|---|---|---|---|
| Sequence | Period 1 | | Period 2 | | Period 3 | |
| Group | Day 1 | Days 2-7 | Day 1 | Days 2-7 | Day 1 | Days 2-6 |
| 1 | H | washout | I | washout | J | no treatment |
| 2 | I | washout | J | washout | H | no treatment |
| 3 | J | washout | H | washout | I | no treatment |

The study treatments are as follows:
Treatment H: 20 mg single dose of Formula (III) as capsule formulation, fasting, different dose level compared to Treatment C
Treatment I: 20 mg single dose of Formula (III) as tablet formulation, fasting.
Treatment J: 20 mg single dose of Formula (III) as tablet formulation, nonfasting (high-fat/high-calorie meal).

Part B (Cohorts 6-10) and C (Cohorts 11 and 12) was run in parallel.

### Fasting and Meals

Fasted State Dosing: Study drug were administered at approximately the same time each day with 240 mL of water following an overnight fast (no food or drinks except water) for at least 10 hours.

Subjects continued to fast until after collection of the 4-hour PK sample on days of intensive PK sampling and 2 hours after drug administration on all other days. Additionally, on PK sampling days, subjects were restricted from water consumption 1 hour before until 2 hours after dosing, except for the 240 mL given with the study treatment. A standardized meal will be provided to subjects after the 4-hour postdose PK blood draw.

Nonfasting State Dosing: Study drug were administered with food and with 240 mL of water. A breakfast was initiated 30 minutes prior to study drug administration (daily dosing or AM dose of twice daily dosing). The PM dose of twice daily dosing was administered 30 minutes after starting an evening snack. The doses were administered at or within 5 minutes of subjects completing the specified meal.

For Part A Cohort 3 Day 8 and Part B of the study (except for Cohort 10, which were dosed under fasting condition), the standardized meal contained approximately 600 to 700 kcal with approximately 30% of the calories from fat.

For Part C of the study, the high-fat/high-calorie breakfast contained approximately 1000 kcal with approximately 50% of the calories from fat.

The evening prior to PK assessment days, subjects underwent an overnight fast (no food or liquids, except water, for at least 10 hours) prior to drug administration (in fed state as described above) the next day. Subjects fasted until after collection of the 4-hour PK sample, relative to study drug administration on PK assessment days and 2 hours after study drug administration on all other days. On days of PK assessments, other than the water provided with dosing and beverages provided with the standardized meal (where applicable), water and other fluids will be withheld for 1 hour before until 2 hours after dose administration. Water may be consumed by subjects following the 2-hour blood draw for the remainder of the collection period. A standardized meal was provided to subjects following morning dosing after the 4-hour post dose blood draw.

All meals and/or snacks given to subjects during their stay in the clinical study facility were standardized for all subjects and should be similar in calorie and fat content and taken at approximately the same time each day. All meals provided were approved by the sponsor. Components of meals (eg, margarine, jelly, bread) were given to subjects in individual portions (eg, 1 tablespoon) per the approved meal schedule. The provision of meal components in bulk (eg, a jar of jelly for subjects to share) was not practiced. All meals should be given at approximately the same time each day (eg, 07:30, 12:00, 18:00).

### Treatment Assessments

### Pharmacokinetic Assessments

### Plasma PK Collection

Formula (III) is a prodrug of Formula (I). Plasma concentrations of Formula (III) metabolite was determined, and PK parameters estimated. Plasma concentrations of Formula (III) and/or its metabolites were determined, and PK explored, as appropriate.

Every effort was made to collect PK samples at the scheduled time point. For all cohorts, the sampling window is ±10% for all PK sampling time points up to 5 hours postdose and ±30 minutes for all PK sampling time points after 5 hours postdose. For all cohorts, a single PK sample was collected at ET, if applicable.

Intensive PK sampling occurred relative to the morning dose of Formula (III) or PTM Formula (III) at the following time points:

### Part A (Cohorts 1 and 2; SAD)

Day 1: predose (≤10 min prior to dosing), 0.5, 1, 2, 3, 4, 6, 9, 12, 18, 24, 36, 48, 72, and 96 hours postdose

### Part A (Cohort 3; SAD)

Day 1 and Day 8: predose (≤10 min prior to dosing), 0.25, 0.5, 1, 2, 3, 4, 6, 9, 12, 18, 24, 36, 48, 72, and 96 hours postdose

### Part A (Cohorts 4 and 5; SAD)

Day 1: predose (≤10 min prior to dosing), 0.25, 0.5, 1, 2, 3, 4, 6, 9, 12, 18, 24, 36, 48, 72, and 96 hours postdose

### Part B (Cohorts 6-10; MAD) For Twice Daily Dosing

Day 1: predose AM (≤10 min prior to dosing), 0.25, 0.5, 1, 2, 3, 4, 6, 9, and 12 hours after.
AM dose (prior to PM dose).
Day 14: predose AM (≤10 min prior to dosing), 0.25, 0.5, 1, 2, 3, 4, 6, 9, 12 (prior to PM
dose), 12.25, 12.5, 13, 14, 15, 16, 18, 21, 24, 36, 48, 72, and 96 hours after AM dose.
A trough (predose) sample will be collected prior to AM dose on Days 3, 4, 5, 9, and 13. OR

### For Daily Dosing.

Day 1: predose (≤10 min prior to dosing), 0.25, 0.5, 1, 2, 3, 4, 6, 9, 12, 18, and 24 hours postdose.
Day 14: predose (≤10 min prior to dosing), 0.25, 0.5, 1, 2, 3, 4, 6, 9, 12, 18, 24, 36, 48, 72, and 96 hours postdose

A trough (predose) sample will be collected prior to dose on Days 3, 4, 5, 9, and 13.

Every effort was made to collect PK samples at the scheduled time point. For all cohorts, the sampling window is ±10% for all PK sampling time points up to 5 hours postdose and ±30 minutes for all PK sampling time points after 5 hours postdose.

For all cohorts, a single PK sample was collected at ET, if applicable.

### Part C (Relative BA/Food Effect/ARA Effect

Cohort 11: Period 1 Day 1, Period 2 Day 1, Period 3 Day 1, and Period 4 Day 6: predose (≤10 min prior to dosing), 0.25, 0.5, 1, 2, 3, 4, 6, 9, 12, 18, 24, 36, 48, 72, and 96 hours postdose.
Cohort 12: Period 1 Day 1, Period 2 Day 1, and Period 3 Day 1: predose (≤10 min prior to dosing), 0.25, 0.5, 1, 2, 3, 4, 6, 9, 12, 18, 24, 36, 48, 72, 96, and 120 hours postdose.

### Pharmacokinetic (PK) Study

Formula (III) is a prodrug and undergoes a rapid conversion to its active metabolite (Formula (I)). The median metabolite to parent ratios after single dose of Formula (III) (20-1000 mg) ranged from 141 to 761 for AUC_{inf}, and 55.2 to 151 for Cₘₐₓ. The median metabolite to parent ratios after once-daily dosing of Formula (III) (20-, 80-, 200-, 500-mg capsule and 200 mg tablet) ranged from 87.1 to 247 for AUCₜₐᵤ, and 29.4 to 129 for Cₘₐₓ on Day 14. Overall, plasma exposures of the prodrug Formula (III) were negligible compared with the active metabolite.

The study characterized the pharmacokinetics (PK) parameters of Formula (III) and the metabolite following single oral doses (20-1000 mg) or multiple oral doses (20-500 mg once daily for 14 days) of Formula (III) in healthy volunteer (Table 9). Formula (III) was administered with liquid-filled capsules or tablets under fasting or non-fasting conditions. The relative bioavailability of tablets versus liquid-filled capsules, and the effect of food on plasma exposure was also characterized.

Concentrations of Formula (III) and the metabolite, in plasma samples were determined using fully validated high-performance liquid chromatography-tandem mass spectrometry (LC-MS/MS) bioanalytical methods. All samples were analyzed in the timeframe supported by frozen stability storage data. The assays for Formula (III), and the metabolite were performed using methods validated by QPS, LLC (Newark, DE, USA).

TABLE 9
Pharmacokinetic Parameters for Each
Analyte and Sample Matrix

| | Sample | |
|---|---|---|
| Analyte | Matrix | Parameters |
| Formula | Plasma | AUCₗₐₛₜ, AUC_{inf} (SD)^{a}, AUCₜₐᵤ (multiple dose), |
| (III) | | Cₘₐₓ, % AUCₑₓₚ (SD), CL/F (SD), CL_{SS}/F |
| | | (multiple dose), t_{1/2}, V_{Z}/F, Tₘₐₓ, C₂₄ |
| | | (multiple dose), Cₜₐᵤ (multiple dose), AUC₀₋₂₄, |
| | | Cₗₐₛₜ, Tₗₐₛₜ, λ_{Z}, AUCₗₐₛₜ/D (SD), AUC_{inf}/D (SD), |
| | | AUC₀₋₂₄/D, AUCₜₐᵤ/D (multiple dose), and |
| | | Cₘₐₓ/D, if applicable |
| Metabolite^{b} | Plasma | AUCₗₐₛₜ, AUC_{inf} (SD)^{a}, AUCₜₐᵤ (multiple dose), |
| | | C₂₄ (multiple dose), Cₜₐᵤ (multiple dose), |
| | | AUC₀₋₂₄, Cₘₐₓ, % AUCₑₓₚ (SD), t_{1/2}, Tₘₐₓ, |
| | | Cₗₐₛₜ, Tₗₐₛₜ, λ_{Z}, AUCₗₐₛₜ/D (SD), AUC_{inf}/D (SD), |
| | | AUC₀₋₂₄/D, AUCₜₐᵤ/D (multiple dose), and |
| | | Cₘₐₓ/D, if applicable |
| | | Metabolic to parent ratio^{c} of AUC and Cₘₐₓ |
| | | for Metabolite to Formula (II), if applicable |

PK = pharmacokinetic(s);
SD = single dose
^{a}Parameter used in sample size and power calculations for Cohorts 11 and 12.
^{b}Metabolic PK parameters may be derived based on the nominal dose of Formula (III).
^{c}Metabolite to parent ratio is weight-normalized, which will be calculated by dividing the molecular weight ratio of metabolite to Formula (III) (ie, 680.62/708.67 = 0.9960).

### Analysis of Relative Bioavailability, Food Effect, Acid-Reducing Agent Effect

The statistical comparisons of the natural log-transformed PK parameters (AUC_{inf}, AUCₗₐₛₜ, and Cₘₐₓ) for each analyte (Formula (III) [if applicable] and Metabolite) and treatment comparison of interest were performed using parametric (normal theory) mixed-effects analysis of variance models. The statistical modeling was based on the PK Analysis Set for the analyte under evaluation. For each analyte, all participants with available data for the PK parameter under evaluation were included in the modeling.

Treatment comparisons of interest are shown in Table 10.

TABLE 10
Statistical Comparisons for Pharmacokinetic Analyses

| | | | Comparison | |
|---|---|---|---|---|
| Cohort | Analytes | Parameter | Test | Reference |
| 3 | Formula | AUC_{inf}, | 200 mg of Formula (III) | 200 mg of Formula |
| (Food Effect) | (III) | AUCₗₐₛₜ, | on Day 8, nonfasting (B) | (III) on Day 1, |
| | | Cₘₐₓ | | fasting (A) |
| 11 | Formula | AUC_{inf}, | 200 mg of Formula | 200 mg of Formula |
| (Bio- | (III) | AUCₗₐₛₜ, | (III) as tablet | (III) as capsule |
| availability) | | Cₘₐₓ | formulation, fasting (D) | formulation, |
| | | | | fasting (C) |
| 11 | Formula | AUC_{inf}, | 200 mg of Formula | 200 mg of Formula |
| (Food Effect) | (III) | AUCₗₐₛₜ, | (III) as tablet | (III) as tablet |
| | | Cₘₐₓ | formulation, nonfasting, | formulation, |
| | | | high-fat/high-calorie meal (E) | fasting (D) |
| 11 | Formula | AUC_{inf}, | 40 mg omeprazole followed | 200 mg of Formula |
| (ARA Effect) | (III) | AUCₗₐₛₜ, | 2 hours later by 200 mg single | (III) as tablet |
| | | Cₘₐₓ | dose of Formula (III) as tablet | formulation, |
| | | | formulation, fasting (G) | fasting (D) |
| 12 | Formula | AUC_{inf}, | 20 mg of Formula | 20 mg of Formula |
| (Bio- | (III) | AUCₗₐₛₜ, | (III) as tablet | (III) as capsule |
| availability) | | Cₘₐₓ | formulation, fasting (I) | formulation, |
| | | | | fasting (H) |
| 12 | Formula | AUC_{inf}, | 20 mg of Formula | 20 mg of Formula |
| (Food Effect) | (III) | AUCₗₐₛₜ, | (III) as tablet | (III) as tablet |
| | | Cₘₐₓ | formulation, nonfasting, | formulation, |
| | | | high-fat/high-calorie | fasting (I) |
| | | | meal (J) | |

ARA = acid-reducing agent

Analysis of dose proportionality, accumulation ratio, and time to steady state was performed.

The metabolite plasma PK parameters after administration of a single dose of Formula (III) (20-, 60-, 200-, 500-, or 1000-mg; capsule formulation) under fasting conditions are presented in Table 11.

Following single oral doses of Formula (III) (20-1000 mg, capsule formulation) under fasting conditions, the median Tₘₐₓ of the metabolite ranged from 0.75 to 3.0 hours. The median t_{1/2} of the metabolite for the higher doses (200-1000 mg) ranged from 9.74 to 15.4 hours, whereas the median t_{1/2} for 20 mg and 60 mg were 1.54 hours and 4.72 hours, respectively, which reflected the distribution phase since the metabolite plasma concentrations fell below the bioanalytical assay limit of quantitation prior to the terminal phase.

TABLE 11
Metabolite Mean (% CV) Plasma PK Parameters Following Single Doses of Formula
(III) Under Fasting Conditions (PK Analysis Set; Part A: SAD Cohorts 1-5)

| | | | | | Cohort 3 | | | | | |
|---|---|---|---|---|---|---|---|---|---|---|
| | Cohort 1 | | Cohort 2 | | Formula | | Cohort 4 | | Cohort 5 | |
| | Formula | | Formula | | (III) | | Formula (III) | | Formula (III) | |
| | (III) 20 mg | | (III) 60 mg | | 200 mg SD | | 500 mg SD | | 1000 mg SD | |
| Metabolite PK | SD Capsule | | SD Capsule | | Capsule | | Capsule | | Capsule | |
| Parameter | Fasting | | Fasting | | Fasting | | Fasting | | Fasting | |
| (Mean | (N = 8) | | (N = 8) | | (N = 8) | | (N = 8) | | (N = 8) | |
| [% CV]) | Day 1 | | Day 1 | | Day 1 | | Day 1 | | Day 1 | |
| AUC_{inf} | 39.6 | (37.1) | 97.3 | (40.3) | 885 | (61.6) | 2400 | (51.2) | 4170 | (62.5) |
| (ng · h/mL) | | | | | | | | | | |
| AUCₗₐₛₜ | 39.3 | (37.3) | 95.9 | (40.4) | 880 | (61.7) | 2390 | (51.5) | 4140 | (63.2) |
| (ng · h/mL) | | | | | | | | | | |
| Cₘₐₓ (ng/ml) | 28.3 | (44.3) | 53.2 | (48.9) | 280 | (57.4) | 665 | (64.3) | 761 | (49.4) |
| Tₘₐₓ (h)^{a} | 0.750 | (0.500, 1.06) | 1.00 | (0.750, 1.04) | 1.01 | (1.00, 2.00) | 2.53 | (2.00, 3.00) | 3.00 | (2.00, 4.00) |
| t_{1/2} (h)^{a} | 1.54 | (1.44, 2.05) | 4.72 | (3.30, 6.71) | 9.74 | (7.82, 21.2) | 15.4 | (8.57, 19.7) | 15.2 | (9.43, 25.7) |
| AUC_{inf}/D | 1.98 | (37.1) | 1.62 | (40.3) | 4.43 | (61.6) | 4.81 | (51.2) | 4.17 | (62.5) |
| (ng · h/mL/mg) | | | | | | | | | | |
| Cₘₐₓ/D | 1.42 | (44.3) | 0.887 | (48.9) | 1.40 | (57.4) | 1.33 | (64.3) | 0.761 | (49.4) |
| (ng/ml/mg) | | | | | | | | | | |
| MRAUC_{inf}^{a} | 761 | (584, 1050) | 141 | (83.3, 220) | 309 | (244, 382) | 277 | (225, 355) | 256 | (173, 433) |
| MRCₘₐₓ^{a} | 150 | (94.9, 173) | 55.2 | (34.5, 60.4) | 84.6 | (65.8, 201) | 151 | (92.7, 210) | 109 | (91.2, 209) |

% CV = percentage coefficient of variation;
PK = pharmacokinetic(s);
Q1 = first quartile;
Q3 = third quartile;
SAD = single ascending dose;
SD = single dose
^{a}Values presented are median (Q1, Q3).
Means presented are unadjusted arithmetic means.
MRCₘₐₓ is defined as metabolite to parent Cₘₐₓ ratio corrected for molecular weight differences.
MRAUC_{inf} is defined as metabolite to parent AUC_{inf} ratio corrected for molecular weight differences.

The data presents an assessment of dose proportionality following single doses using a power model analysis. In this model, the population slope (90% CI) for Part A (SAD Cohorts 1-5) was 1.24 (1.15, 1.34), 1.25 (1.15, 1.34), and 0.901 (0.796, 1.01) for AUC_{inf}, AUCₗₐₛₜ, and Cₘₐₓ, respectively.

Following single oral doses of Formula (III) capsules under fasting conditions, no large deviation from dose proportionality was observed in the metabolite AUC in Formula (III) dose ranges of 20 to 60 mg, and 200 to 500 mg, whereas the transition between 60 mg and 200 mg appeared to result in a more than dose proportional increase in the metabolite AUC. The transition between 500 mg and 1000 mg doses appeared to result in a less than dose proportional increase in the metabolite AUC.

### Formula (III) Plasma Concentrations and Pharmacokinetic Parameters

In humans, Formula (III), undergoes rapid conversion to its active metabolite. The median metabolite to parent ratios (adjusted for molecular weight differences) ranged from 141 to 761 for AUC_{inf}, and 55.2 to 151 for Cₘₐₓ.

Formula (III) plasma PK parameters after administration of a single dose of Formula (III) (20, 60, 200, 500, or 1000 mg) under fasting conditions are presented in Table 12. Overall, plasma exposures of the prodrug Formula (III) were negligible compared with the active metabolite.

### Multiple-Dose Pharmacokinetics of Formula (III) and the Metabolite

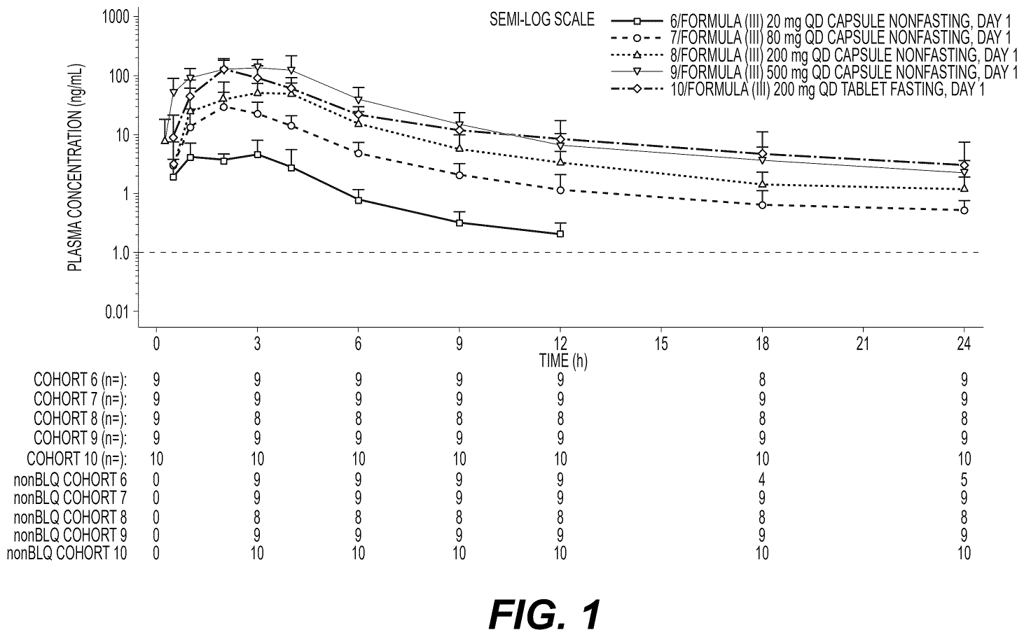

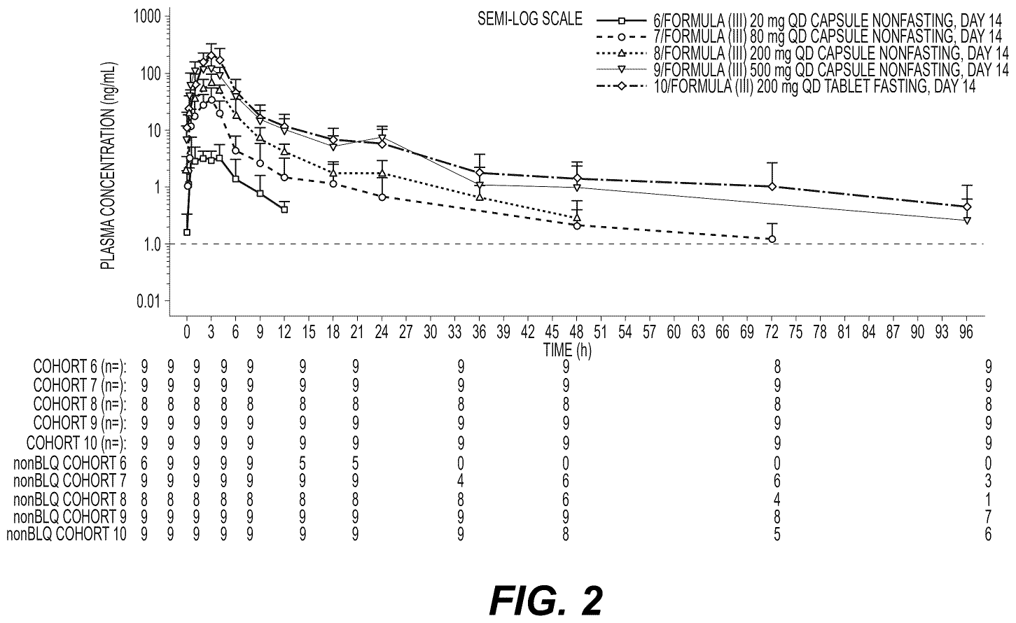

Mean plasma concentration-time profiles after administration of Formula (III) once daily for 14 days under nonfasting conditions (20-, 80-, 200-, and 500-mg capsule formulation [Cohorts 6-9]) and fasting conditions (200-mg tablet formulation [Cohort 10]) are shown in FIG. 1 (Day 1) and FIG. 2 (Day 14). Plasma concentrations of the metabolite increased with increasing Formula (III) dose and were similar or slightly higher after 14 days of dosing compared to the first dose. The terminal phases were approximately parallel across the dose groups.

### Plasma Pharmacokinetic Parameters of the Metabolite

The metabolite's plasma PK parameters after administration of Formula (III) once daily for 14 days under nonfasting conditions (20-, 80-, 200-, and 500-mg capsule formulation [Cohorts 6-9]) and fasting conditions (200-mg tablet formulation [Cohort 10]) are presented in Table 12.

Following multiple doses of Formula (III) capsules under nonfasting conditions or tablets under fasting conditions. The metabolite demonstrated limited accumulation (mean AUCₜₐᵤ accumulation ratio up to 1.49) (Table 13). The median Tₘₐₓ at steady state was between 2 hours and 3 hours. The median t1/2 ranged from 6.90 to 23.0 hours (Table 12).

TABLE 12
The Metabolite's Mean (% CV) Plasma PK Parameters Following Once-Daily Dosing
of Formula (III) for 14 Days Under Nonfasting or Fasting Conditions (The Metabolite's PK
Analysis Set; Part A: MAD Cohorts 6-10)

| | | | | | |
|---|---|---|---|---|---|
| | | Cohort 7 | Cohort 8 | Cohort 9 | Cohort 10 |
| | Cohort 6 | Formula | Formula | Formula | Formula |
| | Formula (III) | (III) 80 mg | (III) 200 mg | (III) 500 mg | (III) 200 mg |
| The metabolite's | 20 mg QD | QD | QD | QD | QD |
| PK Parameter | Capsule | Capsule | Capsule | Capsule | Tablet |
| (Mean | Nonfasting | Nonfasting | Nonfasting | Nonfasting | Fasting |
| [% CV]) | (N = 9) | (N = 9) | (N = 9) | (N = 9) | (N = 10) |

Day 1

| | | | | | | | | | | |
|---|---|---|---|---|---|---|---|---|---|---|
| AUC₀₋₂₄ | 20.2 | (34.7) | 112 | (58.4) | 249 | (24.7) | 670 | (32.7) | 552 | (40.4) |
| (ng · h/mL) | | | | | | | | | | |
| Cₘₐₓ (ng/mL) | 6.63 | (45.1) | 31.6 | (66.0) | 68.4 | (48.5) | 165 | (53.4) | 130 | (52.3) |
| Tₘₐₓ (h)^{a} | 2.00 | (1.00, 3.00) | 2.00 | (2.00, 2.05) | 3.50 | (3.00, 4.00) | 3.00 | (2.00, 3.00) | 2.00 | (2.00, 2.00) |

Day 14

| AUCₜₐᵤ | 24.5 | (57.2) | 142 | (72.7) | 315 | (21.5) | 936 | (33.6) | 728 | (28.2) |
|---|---|---|---|---|---|---|---|---|---|---|
| (ng · h/mL) | | | | | | | | | | |
| Cₘₐₓ (ng/mL) | 5.69 | (72.7) | 37.7 | (55.9) | 78.3 | (31.1) | 249 | (46.4) | 154 | (37.9) |
| Cₜₐᵤ (ng/mL) | —^{a} | | 0.689 | (112) | 1.76 | (67.7) | 6.05 | (75.3) | 7.57 | (55.6) |
| Tₘₐₓ (h)^{b} | 2.00 | (1.00, 4.00) | 2.00 | (2.00, 3.00) | 3.00 | (1.50, 3.50) | 3.00 | (3.00, 4.00) | 1.00 | (1.00, 3.00) |
| t_{1/2} (h)^{b} | 6.90 | (4.69, 8.47) | 23.0 | (18.1, 36.7) | 12.1 | (8.36, 18.8) | 17.4 | (13.3, 18.2) | 20.6 | (13.1, 23.9) |
| AUCₜₐᵤ/D | 1.22 | (57.2) | 1.78 | (72.7) | 1.57 | (21.5) | 1.87 | (33.6) | 3.64 | (28.2) |
| (ng · h/mL/mg) | | | | | | | | | | |
| Cₘₐₓ/D | 0.285 | (72.7) | 0.471 | (55.9) | 0.391 | (31.1) | 0.497 | (46.4) | 0.770 | (37.9) |
| (ng/mL/mg) | | | | | | | | | | |
| MRAUCₜₐᵤ | 191 | (107, 810) | 184 | (115, 198) | 87.1 | (73.3, 133) | 96.1 | (80.3, 102) | 247 | (192, 335) |
| MRCₘₐₓ^{a} | 29.4 | (15.9, 54.2) | 61.7 | (49.9, 88.1) | 48.0 | (28.0, 66.0) | 56.0 | (41.8, 72.9) | 129 | (105, 151) |

BLQ = below the limit of quantitation;
% CV = percentage coefficient of variation;
MAD = multiple ascending dose;
PK = pharmacokinetic(s);
Q1 = first quartile;
Q3 = third quartile;
QD = once daily
^{a}More than one third of postdose values were BLQ, the mean and % CV were not calculated.
^{b}Values are presented as median (Q1, Q3).
One participant in Cohort 8 early terminated from Day 1, 2 hours postdose onwards. One participant in Cohort 10 early terminated from Day 4 onwards.
Means presented are unadjusted arithmetic means.
MRCₘₐₓ is defined as metabolite to parent Cₘₐₓ ratio corrected for molecular weight differences.
MRAUC_{inf} is defined as metabolite to parent AUC_{inf} ratio corrected for molecular weight differences.

TABLE 13
The Metabolite's Mean (% CV) Plasma PK Parameters Accumulation
Analysis (The Metabolite's PK Analysis Set; MAD Cohorts 6-10)

| | Part B (Cohorts 6-10) | | | | |
|---|---|---|---|---|---|
| | Cohort 6 | Cohort 7 | Cohort 8 | Cohort 9 | Cohort 10 |
| | Formula | Formula | Formula | Formula | Formula |
| Metabolite | (III) | (III) | (III) | (III) | (III) |
| PK | 20 mg QD | 80 mg QD | 200 mg QD | 500 mg QD | 200 mg QD |
| Parameter | Capsule | Capsule | Capsule | Capsule | Tablet |
| (Mean | Nonfasting | Nonfasting | Nonfasting | Nonfasting | Fasting |
| [% CV]) | (N = 9) | (N = 9) | (N = 9) | (N = 9) | (N = 10) |
| AR Cₘₐₓ | 0.894 (65.4) | 1.39 (56.2) | 1.34 (45.6) | 1.58 (28.9) | 1.46 (42.9) |
| AR AUCₜₐᵤ | 1.14 (25.2) | 1.23 (24.5) | 1.31 (23.1) | 1.40 (12.7) | 1.49 (32.5) |

AR = accumulation ratio;
% CV = percentage coefficient of variation;
MAD = multiple ascending dose;
PK = pharmacokinetic(s);
QD = once daily
For Study Day 1, AUC₀₋₂₄ values were used for AUCₜₐᵤ and C₂₄ values were used for Cₜₐᵤ.
One participant in Cohort 8 early terminated from Day 1, 2 hours postdose onwards. One participant in Cohort 10 early terminated from Day 4 onwards.

The median metabolite to parent ratios after once-daily dosing of Formula (III) (adjusted for molecular weight differences) ranged from 87.1 to 247 for AUCₜₐᵤ, and 29.4 to 129 for Cₘₐₓ on Day 14 (Table 12).

The PK data based on planned time points and quality checked plasma concentrations are summarized as follows.

In human, Formula (III), undergoes rapid conversion to its active metabolite. Following single doses of liquid-filled capsules under fasting conditions, the median Cₘₐₓ ratio of the metabolite to Formula (III) ranged from 55 to 151 across dose levels. The true terminal elimination phase of the metabolite was characterized with the higher Formula (III) doses of 200 to 1000 mg, with median terminal elimination half-life of 9 to 13 hours. The observed terminal half-life for the 20 and 60 mg doses was 1.4 to 3.7 hours, which reflected the distribution phase rather than the true terminal phase since the concentrations fell below the limit of quantitation prior to reaching the terminal phase. The preliminary PK parameters for metabolite following single doses of Formula (III) capsules under fasting conditions are shown in Table 11.

Following multiple doses of liquid-filled capsules under nonfasting condition, the metabolite demonstrated limited accumulation (AUCₜₐᵤ accumulation ratio up to 1.37). The functional half-life, estimated based on steady state Cₘₐₓ and C_{trough} ratio, ranged from 4 to 5 hours. The metabolite multiple-dose exposure with administration of the 200-mg Formula (III) tablet under fasting conditions was slightly lower than the 500-mg nonfasting capsule multiple-dose exposure, consistent with relative bioavailability and food effect characterized with single-dose evaluation. The preliminary PK parameters for the metabolite following multiple once-daily doses of Formula (III) are shown in Table 12.

The relative bioavailability of Formula (III) tablet compared to capsule was evaluated at both 20 mg and 200 mg doses. The tablet bioavailability relative to the capsule was about 60% at 20 mg and about 90% at 200 mg. Food negatively affected the exposure of the metabolite. With a high-fat/high-calorie meal, the AUC of the metabolite was about 20% to 40% lower compared to that under fasting condition.

### Pharmacodynamic Assessments

Blood samples will be collected to measure PD biomarkers for the active metabolite of Formula (III), at the time points described below, where 0 indicates the predose time point in the morning. Blood samples for PD assessments will be collected in the same subject order on each day, including the day prior to the dosing day (Day −1), in each cohort. The collection time on the day prior to the dosing day (Day −1) time points must match the time of the collection on the day of dosing time points (≤5 minutes).

### Part A (Cohorts 1 and 2; SAD)

Day −1 (≤5 min of Day 1 time point): 0, 1, 3, 6, and 12 hours.
Day 1: predose (≤10 min prior to dosing), 1, 3, 6, 12, 24, 36, 48, 72, and 96 hours postdose.

### Part A (Cohorts 3, 4, and 5; SAD)

Day −1 (≤5 min of Day 1 time point): 0, 1, 3, 6, 9, 12, and 18 hours.
Day 1: predose (≤10 min prior to dosing), 1, 3, 6, 9, 12, 18, 24, 36, 48, 72, and 96 hours postdose.

### Part B (Cohorts 6-9; MAD) for Once Daily Dosing

Day −1 (≤5 min of Days 1, 2, 5, 9, and 14 time points): 0, 3, 6, 9, 12, and 18 hours.
Day 1: predose (≤10 min prior to dosing), 3, 6, 9, 12, and 18 hours postdose.
Days 2, 5, 9: predose (≤10 min prior to dosing) and 6 hours postdose.
Day 14: predose (≤10 min prior to dosing), 6, 9, 12, 18, 24, 36, 48, 72, and 96 hours postdose.

### Part B (Cohort 10; MAD) for Once Daily Dosing

Day −1 (≤5 min of Days 1, 2, 5, 9, and 14 time points): 0, 1, 3, 6, 9, 12, and 18 hours.
Day 1: predose (≤10 min prior to dosing), 1, 3, 6, 9, 12, and 18 hours postdose.
Days 2, 5, 9: predose (≤10 min prior to dosing) and 6 hours postdose.
Day 14: predose (≤10 min prior to dosing), 1, 3, 6, 9, 12, 18, 24, 36, 48, 72, and 96 hours postdose.

### Peripheral Blood Mononuclear Cells and Whole Blood:

### Part B (Cohorts 6-10; MAD) for Twice Daily Dosing

Day −1 (≤5 min of Days 1, 2, 5, 9, and 14 time points): 0 and 6 hours.
Day 1: predose AM (≤10 min prior to dosing) and 6 hours postdose AM.
Days 2, 5, 9:6 hours postdose AM.
Day 14: predose AM (≤10 min prior to dosing), 6, 48, and 96 hours postdose AM. OR

### For Daily Dosing

Day −1 (≤5 min of Days 1, 2, 5, 9, and 14 time points): 0 and 6 hours.
Day 1: predose AM (≤10 min prior to dosing) and 6 hours postdose AM.
Days 2, 5, 9:6 hours postdose AM.
Day 14: predose AM (≤10 min prior to dosing), 6, 48, and 96 hours postdose AM.

### Receptor Occupancy (RO) Assay

Formula (III) is a prodrug of Formula (I) and undergoes a rapid conversion to its active metabolite (Formula (I)) when administered to a human. The pharmacodynamics (PD) of the active metabolite of Formula (III) were analyzed in healthy volunteers by measuring the metabolites receptor occupancy (RO) on α4β7 receptors in whole blood to inform Formula (III) dose selection and schedule.

A whole blood RO assay was developed and fit-for-purpose-validated that measures free α4β7 receptors on 7+CD4+CD45RO+ memory T cells in fresh whole blood by flow cytometry. The β7+CD4+CD45RO+ memory T-cells were identified using a staining panel comprised of fluorescently labelled anti-CD3, -CD4, -CD8, -CD45RO, and -β7 antibodies (Biolegend) (Table 14) as soon as possible after blood collection. Biotinylated human MAdCAM-1 protein, the natural ligand for α4β7, is mixed with streptavidin PE dextramer (Immudex) (MDX) and added to the whole blood in the presence of the divalent cation manganese (as MnCl₂). MDX and the metabolite compete for binding to the α4β7 integrin receptor. Specimen data are acquired immediately on a BD LSRFortessa X-20 Cell Analyzer flow cytometer using a α4β7 RO cell acquisition template and application settings. The gating strategy is as follows: lymphocytes are gated based on size (FSC-A) and complexity (SSC-A). Doublets are removed using FSC-A/FSC-H. CD3+ lymphocytes are further differentiated into CD4+ and CD8+ T cells. CD4+CD45RO+ positive T cells are gated on β7 integrin and MDX positivity. The fluorescent intensities of PE-MDX β7+CD4+CD45RO+ memory T cells in pre-dose and post-dose whole blood are used to calculate the % RO post-dose. The lower level of quantification (LLOQ) of the assay was estimated to be ˜40% for a single measurement; similarly, the a4b7 RO assay was fit-for purpose validated to precisely quantify % RO up to 95% RO. Actual measured values <40% RO and ≥95% RO are shown but represent best estimates.

TABLE 14
Cell surface markers and antibodies a4ß7 RO assay

| | | | | | | |
|---|---|---|---|---|---|---|
| Fluorochrome | AF488 | BV510 | PerCP Cy5.5 | BV421 | APC | PE |
| Marker | CD3 | CD4 | CD8 | CD45RO | β7 | Biotinylated |
| | | | | | | MAdCAM-1 + SA- |
| | | | | | | PE |

Whole blood was collected pre- and post-Formula (III) or placebo to match (PTM) administration and percent α4β7 RO was calculated for all postbaseline time points in the SAD and MAD cohorts of the study. α4β7% RO was evaluated in all participants in Part A (SAD, Formula (III) 20 mg, 60 mg, 200 mg, 500 mg, and 1000 mg administered in liquid filled capsules under fasting conditions), and in Part B (MAD, Formula (III) 20 mg, 80 mg, 200 mg, 500 mg in liquid filled capsules under non-fasting conditions and Formula (III) 200 mg in tablet formulation under fasting conditions). In total, 2,506 samples collected at 35 different time-points from 109 unique participants were analyzed for α4β7% RO.

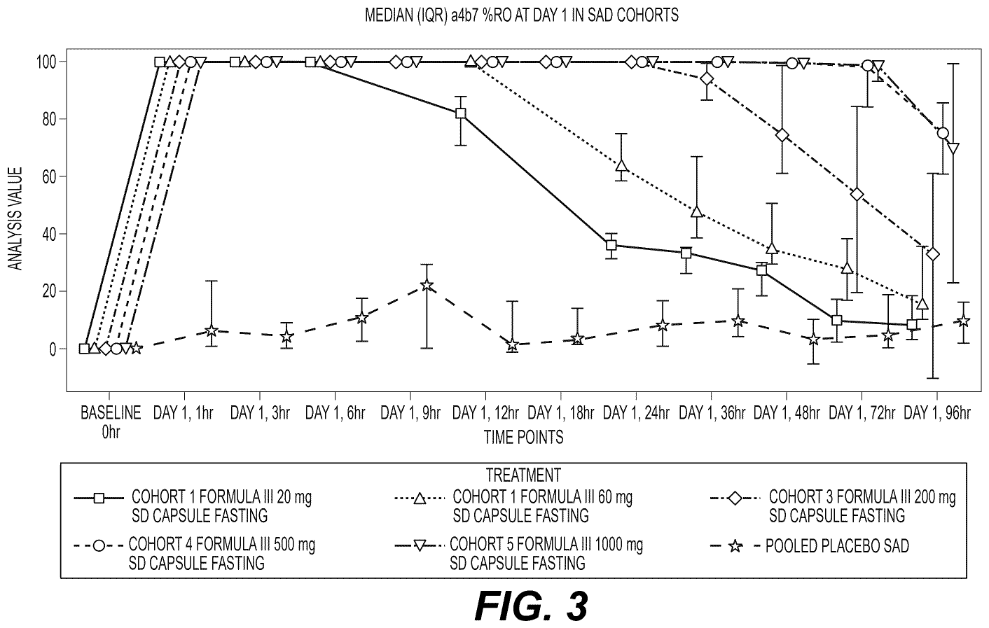

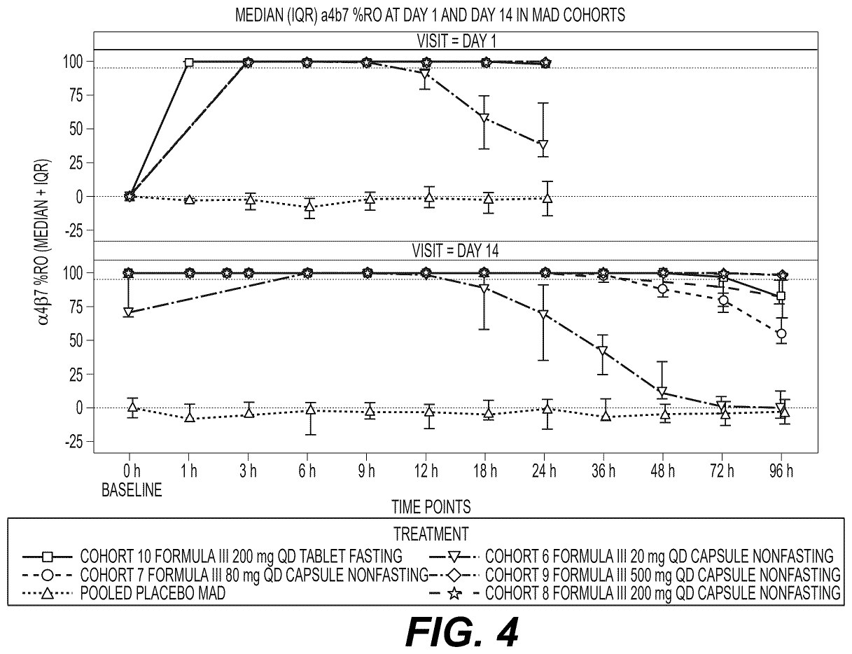

The results of α4β7% RO after single ascending doses Formula (III) in the SAD (Part A) immediately postdose (3 h) and 24 h postdose on Day 1, and after the first dose Formula (III) in the MAD (Part B) immediately (3 h) and 24 h postdose (C_{trough}) on Day 1 and after multiple once daily doses on Day 14 prior to the last dose and 24 h post the last dose on Day 14 are shown in Table 15. FIG. 3 shows the observed α4β7% RO after a single dose on Day 1 in the SAD; FIG. 4 shows the observed α4β7% RO after the first dose on Day 1 and after multiple once daily doses on Day 14 in the MAD. Of note, Cohort 6 Day 1 3 h postdose was not available; Cohort 6-9 Day 14 1 h and 3 h postdose were not collected.

TABLE 15
α4β7 Receptor Occupancy in SAD and MAD Cohorts

| Formula (III) SD | Part A SAD | | | |
|---|---|---|---|---|
| (Fasting, Liquid- | α4β7 % RO (IQR) | | | |
| Filled Capsule | Day 1 | | | |
| Formulation) | 3 h Postdose | | 24 h Postdose | |
| Cohort 1 | 100 | (100, 100) | 35.9 | (33.4, 38.6) |
| 20 mg (N = 8) | | | | |
| Cohort 2 | 100 | (100, 100) | 63.4 | (59.7, 71) |
| 60 mg (N = 8) | | | | |
| Cohort 3 | 99.9 | (99.9, 99.9) | 99.7 | (99.6, 99.9) |
| 200 mg (N = 8) | | | | |
| Cohort 4 | 99.9 | (99.8, 99.9) | 99.8 | (99.8, 99.9) |
| 500 mg (N = 8) | | | | |
| Cohort 4 | 99.6 | (99.5, 99.9) | 99.8 | (99.8, 99.9) |
| 1000 mg | | | | |
| (N = 8) | | | | |
| Pooled Placebo | 4.3 | (0.4, 8.2) * | 8.2 | (1.5, 16) * |
| SAD (N = 10) | | | | |

| Formula (III) QD | Part B MAD | | | | | | | |
|---|---|---|---|---|---|---|---|---|
| (Nonfasting, | α4β7 % RO (IQR) | | | | | | | |
| Liquid-Filled | Day 1 | | | | Day 14 | | | |
| Capsule | 3 h | | 24 h | | | | 24 h | |
| Formulation) | Postdose | | Postdose | | Predose | | Postdose | |
| Cohort 6 | 99.8 | (99.8, 99.9) | 37.8 | (29.5, 69.0) | 70.8 | (67.4, 96.6) | 69.3 | (35, 91.1) |
| 20 mg (N = 9) | | | | | | | | |
| Cohort 7 | 99.9 | (9.7, 99.9) | 98.1 | (97.5, 99.1) | 99.3 | (99.1, 99.7) | 99.4 | (99.1, 99.7) |
| 80 mg (N = 9) | | | | | | | | |
| Cohort 8 | NA | | 99.8 | (99.8, 99.9) | 99.9 | (99.9, 99.9) | 99.8 | (99.8, 99.9) |
| 200 mg (N = 9) | | | | | | | | |
| Cohort 9 | 99.9 | (99.9, 99.9) | 99.9 | (99.8, 99.9) | 99.9 | (99.9, 99.9) | 99.9 | (99.8, 99.9) |
| 500 mg (N = 9) | | | | | | | | |
| Cohort 10 | 99.9 | (99.9, 99.9) | 99.8 | (99.6, 99.9) | 100 | (99.9, 100) | 99.9 | (99.9, 99.9) |
| 200 mg | | | | | | | | |
| (Fasting, Tablet | | | | | | | | |
| Formulation) | | | | | | | | |
| (N = 10) | | | | | | | | |
| Pooled Placebo | −1.9 | (−9.1, 2) * | −1.6 | (−13.5, 8.3) * | −0.3 | (−6.5, 6.3) * | −0.6 | (−10.1, 4.5) * |

After a single dose of Formula (III) administered under fasting conditions in the SAD, full target engagement of α4β7 (% RO≥95) was achieved immediately postdose (3 h) for all doses tested and was sustained 24 h postdose in 100% of subjects post Formula (III) doses ≥200 mg supporting the once daily dosing schedule in the MAD part of the study. % RO analyses of α4β7 in the MAD confirmed results observed in the SAD on Day 1 and additionally demonstrated that 80 mg Formula (III) under nonfasting conditions achieved full target engagement of α4β7 at 24 h on Day 1. Analyses additionally indicated an accumulation of α4β7% RO post multiple once daily doses as demonstrated post 20 mg Formula (III) from Day 1 to Day 14 (Table 15). % RO≥95% was sustained 24 h post the last once daily dose Formula (III) on Day 14 for all doses ≥80 mg in 100% of participants. FIG. 4 additionally shows that α4β7% RO≥95% post the last dose on Day 14 was sustained until 72 h post the last dose 200 mg Formula (III) administered under fasting conditions in tablet formulation, and until 96 h post last dose 500 mg Formula (III) administered under nonfasting conditions in capsule formulation in 100% of participants. Day 14 36 h and 48 h postdose α4β7% RO data were not available for the 200 mg capsule formulation under nonfasting condition. α4β7% RO at 24 h post last dose on Day 14 in this cohort was ≥95% in all participants and on average 90.1% 72 h postdose, suggesting full target engagement could possibly be achieved until approximately 48 h with 200 mg under nonfasting conditions in capsule formulation.

### PK-RO Modelling to Inform Dose Selection

A preliminary population PK model, using PK data from Cohorts 1 through 9 and Cohort 11 with doses ranging from 20 mg to 1000 mg was developed to characterize the PK of the metabolite, quantify sources of PK variability, estimate concentrations input for PD analyses, and predict exposures for the tablet formulation dose for Example 4 and 5. Pharmacodynamic assessments included measuring the metabolite target α4β7 RO in whole blood. Preliminary RO data from Cohorts 1 through 8 were used to develop the PK-PD models, describing the correlation of the metabolite concentrations and RO. A biophase (effect) compartment PD model was implemented to characterize the observed prolonged RO on α4β7. The PK-RO model described the observed data well based on visual inspection and diagnostic plots.

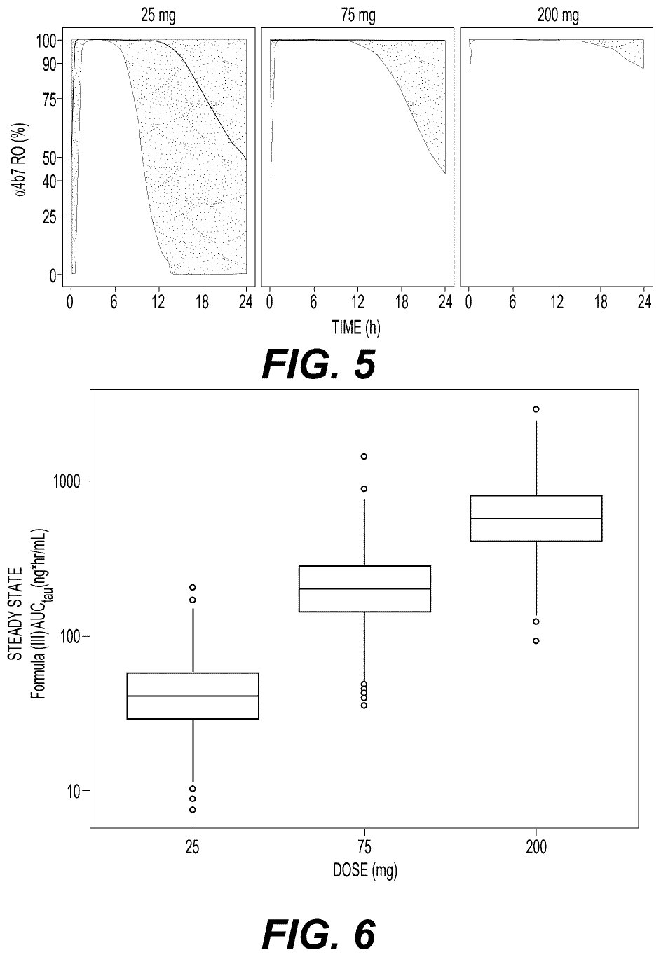

Using the developed population PK-PD models, the PK and RO for potential Experiment 4 doses were simulated. The simulated RO profiles for the Experiment 4 dosages, (Part 1) of 25 mg, 75 mg, and 200 mg once-daily Formula (III) tablet, under fasting conditions, are shown in FIG. 5. The simulated profiles for α4β7 RO suggest that 25 mg once daily is likely a subtherapeutic dose, with median duration of full target engagement (% RO≥95%) for about 13 hours, whereas 75 mg once daily represents a potential therapeutic dose with median duration of full target engagement (% RO≥95%) over the 24-hour dosing interval, and 200 mg once daily is potentially a high therapeutic dose, which is included to characterize the plateau of clinical responses. In terms of PK for the selected Experiment 4 doses, the simulated AUCₜₐᵤ at steady state is shown in FIG. 6, which suggests adequate exposure separation for exposure-response characterization in Experiment 4. Taken together, the PK-PD simulation supports the evaluation of 25 mg, 75 mg, and 200 mg once daily in participants with UC in the Experiment 4 dose-ranging study.

### Example 2. Efficacy of Formula (III) in a Mouse IL10−/− Knockout Colitis Model

The following study was conducted to evaluate the efficacy of an α4β7 integrin small molecule inhibitor relative to no treatment and to an IL-12/IL-23 inhibitor (anti-p40 antibody a murine surrogate of ustekinumab). At 12 weeks colitis phenotype was confirmed when the mice showed visible signs of colitis as determined by mouse colonoscopy. Animals that exhibited colitis at the 12 weeks old were entered into the study and treated for three weeks (21-days total) In life analysis for tracking colitis included daily body weights, daily stool frequency, daily stool consistency, and endoscopic colitis assessments. Endpoint analysis included measurement of colon lamina propria lymphocytes, and histology scoring of the colon tissue.

12-week-old IL10−/− knockout C57CL/6 which showed clinical features of colitis, including weight loss and increased stool frequency, where used for the experiments. Mice were treated for three (3) weeks with Formula (III) mixed with food chow (0.125%), and 4 mg/kg anti-p40 dosed intraperitoneally every four (4) days (QW).

Formula (III). Efficacy of Formula (III) in a Mouse IL10−/− knockout Colitis Model

### Methods

### Animals

Female IL-10 deficient C57CL/6 mice (aged 12 weeks at study inception) were used in this study. All animal experiments were conducted according to Swiss animal well fare law and approved by the local animal welfare authority of Zurich County (Tierversuchskommission Zürich, Zurich, Switzerland, License No. ZH256-2014).

### In Life Experimental Protocol for Colitis Treatment

The experimental design is shown in Table 16. Mice were administered either standard chow diet (Harlan Teklad Global Diets 2014, TD2014) or standard chow diet plus 0.125% of in chow. Formula (III) schedule was ad libitum while the Anti-p40 antibody was administered via intraperitoneal injection on day 1 and then every four days for three weeks.

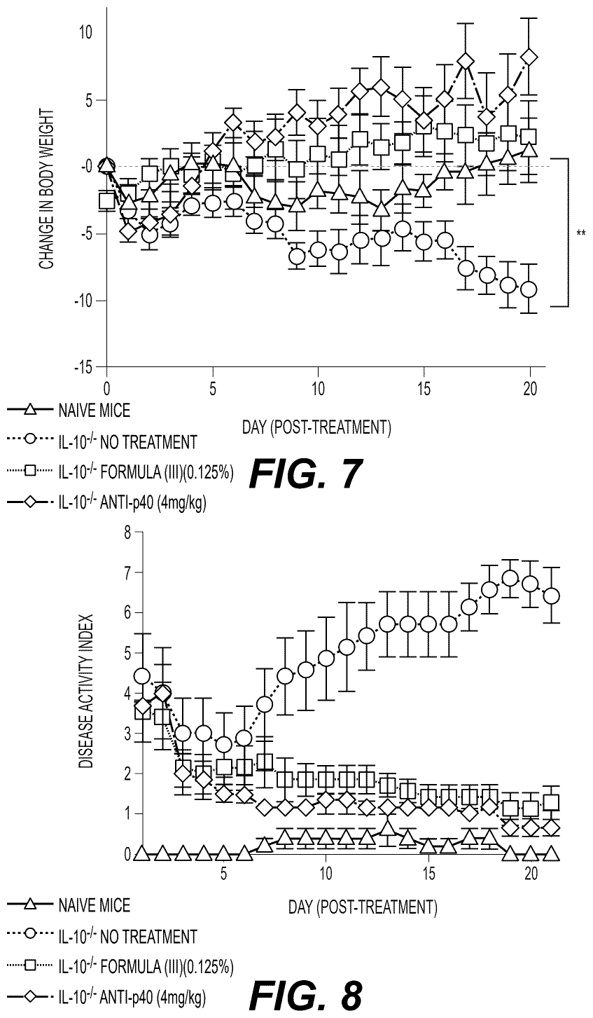

The compound was added and mixed into the chow powder at 0.125% concentration, anti-p40 antibody was diluted in phosphate buffered saline (2 mg/mL) (anti-p40 antibody, a murine surrogate of ustekinumab) then dosed at 4 mg/kg every four days by intraperitoneal injection. Starting with 12 weeks old mice which displayed macroscopically visible signs of colitis as evidenced by mouse colonoscopy were used for the experiments. The mice were then treated for three (3) weeks (21-days). On Day 21, mice were sacrificed, blood and tissue samples were collected for further analysis. While untreated IL-10−/− knockout mice progressively lost body weight and manifested colitis activity (measured by weight loss, stool frequency and stool consistency). The α4β7 small molecule inhibitor (Formula (III) compound) ameliorated body weight loss, FIG. 7, and reduced disease activity, FIG. 8, similar to the anti-p40 antibody.

TABLE 16
Experimental Design and Dose Groups

| Group | N | Mice | Compound | Dose (mg/kg) | Route | Schedule |
|---|---|---|---|---|---|---|
| 1 | 10 | WT | Normal Chow | NA | Chow | Ad libitum |
| | | littermates | | | | |
| 2 | 10 | KO | Normal Chow | NA | Chow | Ad libitum |
| 3 | 10 | KO | α4β7i | 0.125% | Chow | Ad libitum |
| | | | Formula (III) | | | |
| 4 | 10 | KO | Anti-p40 | TBD | IP | QW |

### Assessment of Colitis by Endoscope

Mice were anesthetized with and mixture of ketamine (90-120 mg/kg-1 body weight, Vétoquinol, Bern, Switzerland) and Xylazine (8 mg/kg-1 body weight, Bayer, Lyssach, Switzerland) by intraperitoneal injection. Colonoscopies were performed as described in Becker C, Fantini M C, Neurath M F. High resolution colonoscopy in live mice. Nat. Protoc, 2006; 1:2900-4. The procedure was recorded with a Karl Storz Tele Pack Pal 20,043,020 (Karl Storz Endoskope, Tuttlingen, Germany). Scoring of the colonoscopy was done in accordance with the Murine Endoscopic Index of Colitis Severity (MEICS) scoring system as described in Becker C., Fantini M. C., Neurath M. F. High resolution colonoscopy in live mice. Nat. Protoc. 2006; 1:2900-2904. doi: 10.1038/nprot.2006.446. The following five (5) parameters were assessed: (1) transparency of the colon; (2) changes of the vascular pattern; (3) fibrin visible; (4) granularity of the mucosal surface; and (5) stool consistency.

### Histological Score

Tissue Extraction: Mouse colons were extracted and fixed in 4% formalin, embedded in paraffin and then cut into 5-μm sections. The colon sections where then deparaffinized and stained with hematoxylin and eosin. Upon completion of the staining histological scoring was performed as shown in Table 17 below.

TABLE 17

| Epithelium | | Infiltrate | | Mucosa | |
|---|---|---|---|---|---|
| | score | | score | | score |
| Normal morphology | 0 | No infiltrate | 0 | | 0 |
| Minimal hyperplasia | 1 | Around crypt base | 1 | 1-2 ulcerations (≤20 | |
| in <10% | | | | crypts in total) | |
| Mild hyperplasia | 2 | Infiltration of L. | 2 | 1-4 Ulcerations | 2 |
| (~2× thicker), goblet | | muscularis | | (>20-40 crypts | |
| cell loss (±cryptitis, | | mucosae | | in total) | |
| ±erosions) in | | | | | |
| 10-30% | | | | | |
| Moderate | 3 | Marked edematous | 3 | Any group of ulcers | 3 |
| hyperplasia (2-3× | | infiltration of L. | | that exceeds the | |
| thicker), goblet cell | | muscularis | | criteria above | |
| loss (cryptitis, ±crypt | | mucosae | | | |
| abscesses) in | | | | | |
| 40-70% | | | | | |
| Marked hyperplasia | 4 | Infiltration of Tela | 4 | | |
| (≥4× thicker), goblet | | submucosa | | | |
| cell loss (multiple | | | | | |
| crypt abscesses) in | | | | | |
| >70% | | | | | |

### Isolation of Colon Lamina Propria Lymphocytes

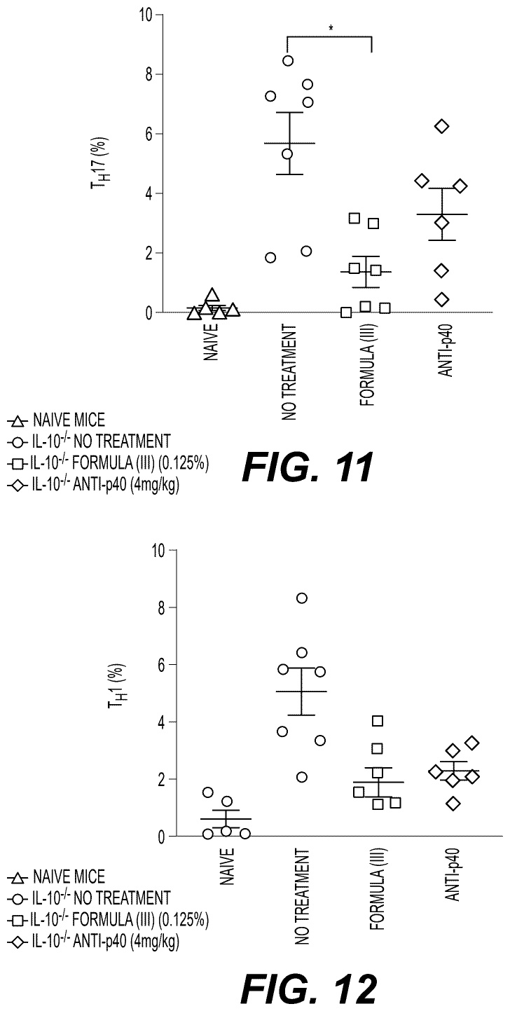

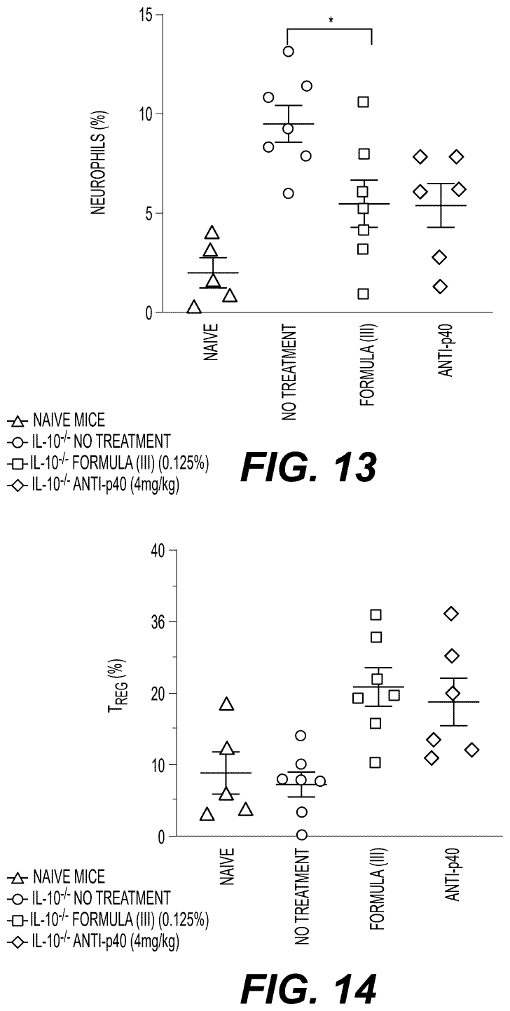

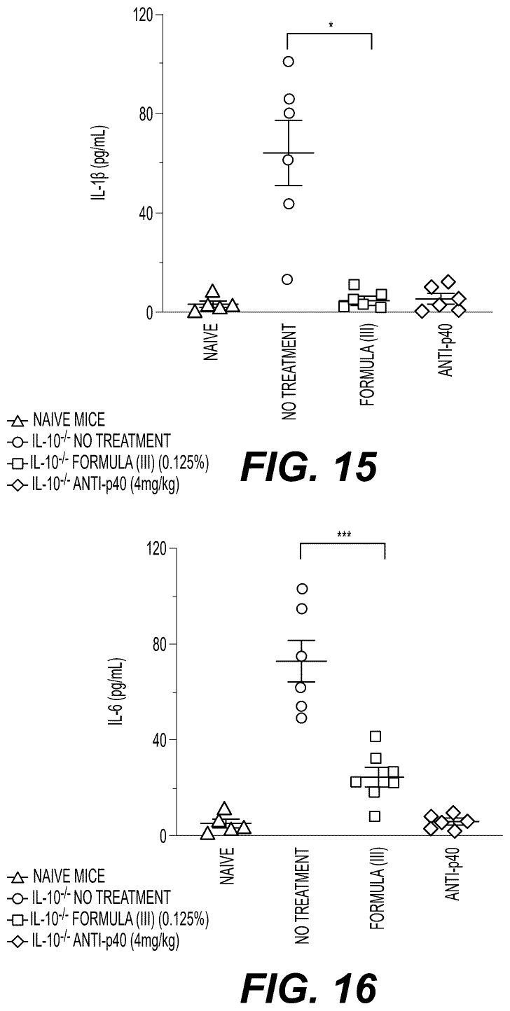

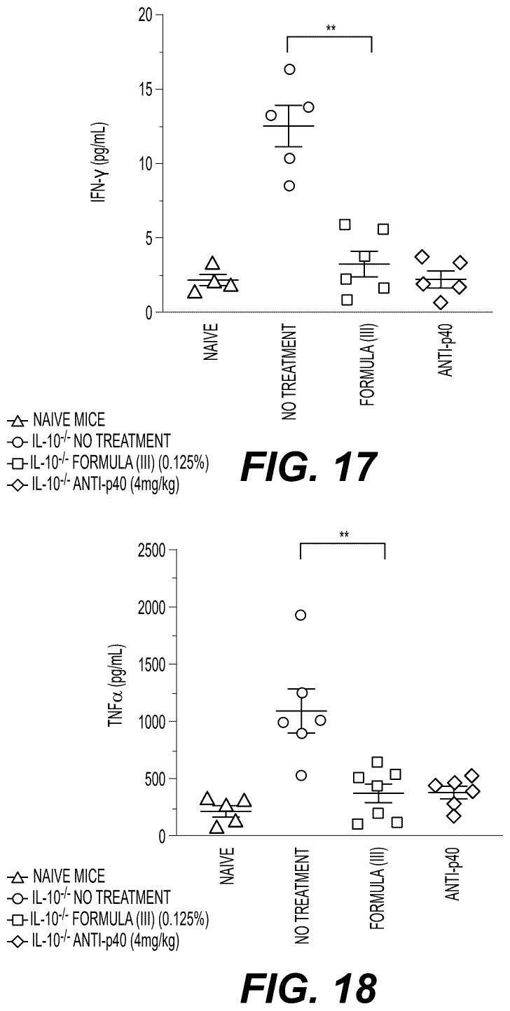

Mouse colons were extracted and opened up in a longitudinal orientation, placed in a 15 mL falcon tube, rinsed with PBS (Gibco, Thermo Fisher Scientific, Carlsbad, CA, USA). The colon was then cut into 2 mm² sized samples and washed with 2% FBS HBSS (Thermo Fisher Scientific). The supernatant was discarded, resuspended in HBSS supplemented with 2 mM EDTA (Thermo Fisher Scientific) and then incubated at 37° C. on a shaker (300 rpm) for 15 minutes. Upon completion of the incubation the tubes were vigorously shaken, the supernatant was carefully discarded, and the sample was washed with HBSS. Next, the samples were resuspended in a digestion buffer consisting of Dispase (0.6 mg/mL, Gibco) supplemented Collagenase IV (0.4 mg/mL; Clostridium Histolyticum, Sigma-Aldrich). The solution was incubated for 20 minutes on a 37° C. shaker (300 rpm). Upon completion, the samples were homogenized by passing the solution through a 18+1.5-gauge needle. Samples where then stained with T_{H}1, T_{H}17, Neutrophils, and T_{reg} antibodies followed by preforming Flow cytometry analysis of the mice immune cells of the colon tissue. Formula (III) significantly reduced the T_{H}1, T_{H}17, and neutrophils without affecting the frequency of the T_{reg} FIGS. 11-14.

### Results

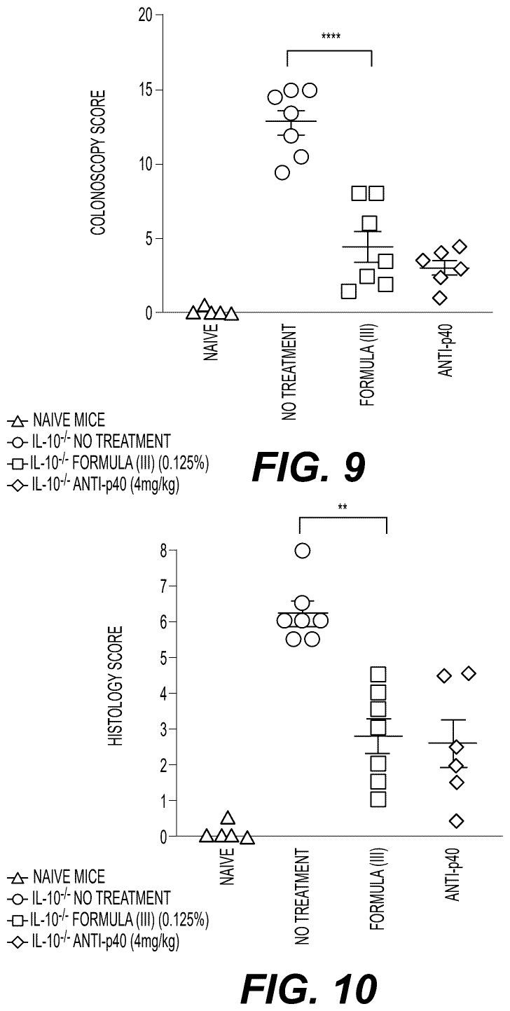

Formula (III) significantly improved the colonoscopy score, similar to the anti-p40 antibody, over the non-treatment group FIG. 9. Formula (III) also showed a reduced histopathology score, similar to the Anti-p40 antibody, compared to the non-treatment group FIG. 10. Reduction of inflammatory T-cells and neutrophils in the tissue of the mice colon is also consistent with reduced proinflammatory cytokines detected in the blood. Finally, we found that treating colitis with a single agent such Formula (III) or anti-p40 antibody significantly ameliorates intestine inflammation.

### Example 3. Formula (III) α4β7 Integrin Small Molecule Inhibitor and Anti-p40 Combination Treatment Achieved Superior and Synergistic Efficacy as Compared with Either Pathway Alone

The following experiments were run to evaluate the effects of a combination treatment of Formula (III) in combination with an anti-p40 antibody on the IL-10−/− mouse colitis model compared to the single treatment alone.

### Animals

Female IL-10 deficient C57CL/6 mice (aged 12 weeks at study inception) were used in this study. All animal experiments were conducted according to Swiss animal well fare law and approved by the local animal welfare authority of Zurich County (Tierversuchskommission Zürich, Zurich, Switzerland, License No. ZH256-2014).

### In Life Experimental Protocol for Colitis Treatment

The experimental design is shown in Table 18. Mice were administered either standard chow diet (Harlan Teklad Global Diets 2014, TD2014) or standard chow diet plus 0.005% of Formula (III) in the chow. Formula (III)'s schedule was ad libitum while the anti-p40 antibody was administered via an intraperitoneal injection on day 1 and then every four days for three weeks.

The compound was added and mixed to the chow powder at 0.125% concentration, anti-p40 antibody was diluted in phosphate buffered saline (2 mg/mL) (anti-p40 antibody is a murine surrogate of ustekinumab) then dosed at either 2 mg/kg (low dose) or 4 mg/kg (high dose) every four days by intraperitoneal injection. Starting with 12 weeks old mice which displayed macroscopically visible signs of colitis as evidenced by mouse colonoscopy were used for the experiments. The mice were then treated for three (3) weeks (21-days). On Day 21, mice were sacrificed, blood and tissue samples were collected for further analysis.

TABLE 18

| | | | | Dose | | | |
|---|---|---|---|---|---|---|---|
| | | | | (% in | | | |
| | | | | chow) or | | | |
| Group | N | Mice | Compound | mg/Kg | | Route | Schedule |
| 1 | 5 | WT | Normal Chow | NA | | Chow | Ad libitum |
| | | littermates | | | | | |
| 2 | 5 | KO | Normal Chow | NA | | Chow | Ad Libitum |
| 3 | 6 | KO | Formula (III) | 0.005% in chow | | chow | Ad Libitum |
| 4 | 6 | KO | Formula (III) + | 0.005% in | | chow + | Ad Libitum + |
| | | | Anti-p40 | chow + 0.2 mg/Kg | | IP | QW |
| 5 | 6 | KO | Anti-p40 | 0.2 | mg/Kg | IP | QW |
| 6 | 6 | KO | Anti-p40 | 4 | mg/Kg | IP | QW |

### Assessment of Colitis by Endoscope

Experiments were conducted as describe in Experiment 2.

### Histological Score

Experiments were conducted as describe in Experiment 2.

### Isolation of Colon Lamina Propria Lymphocytes

Experiments were conducted as describe in Experiment 2.

### Results

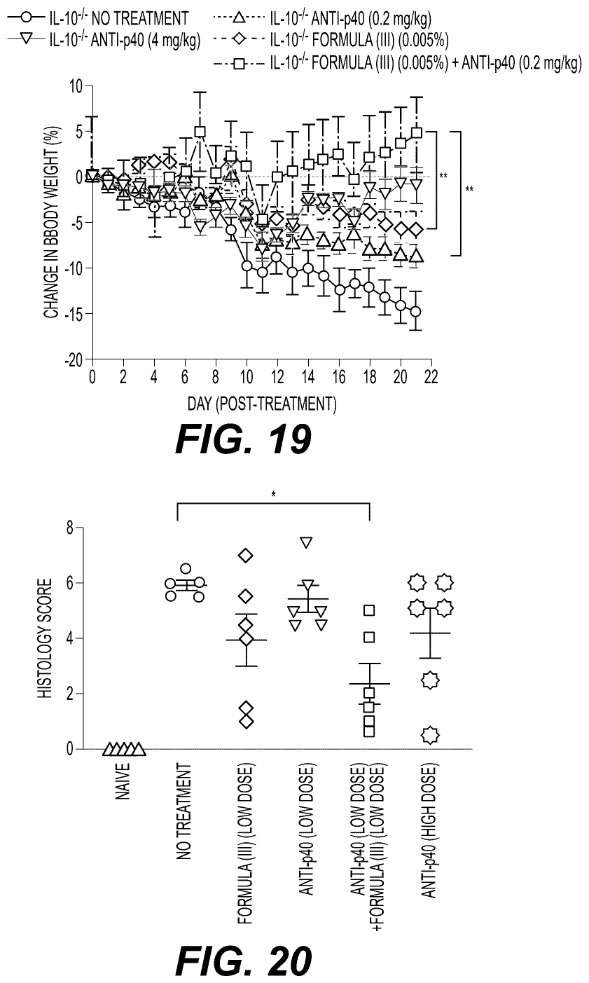

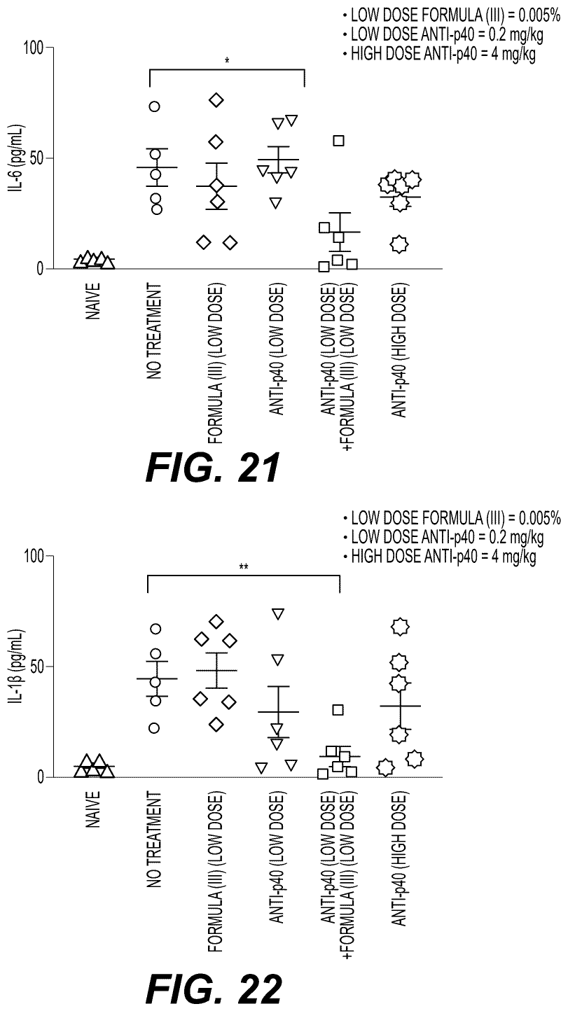

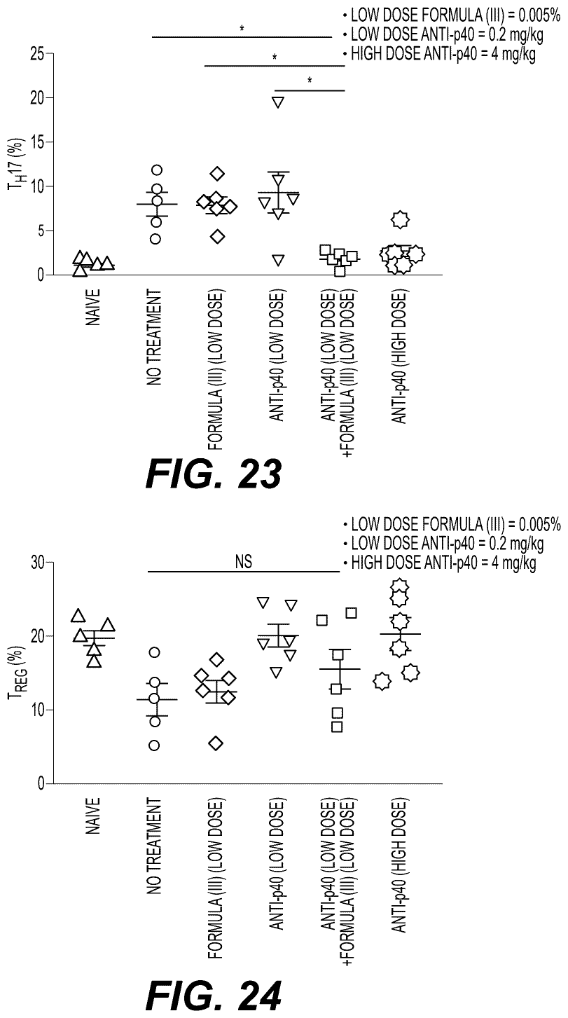

Treatment with Formula (III) or an anti-p40 alone leads to improved body weight loss as compared with no treatment control, however the combination treatment with of Formula (III) and anti-p40 antibody showed superior improvement of body weight loss, FIG. 19, and histological score, FIG. 20 as compared either pathway alone. The combination of Formula (III) and anti-p40 antibody led to a significant reduction of serum proinflammatory cytokines including IL-6 FIG. 21, and IL-1B, FIG. 22. In addition, the combination Formula (III) and anti-p40 antibody led to superior inhibition of T_{H}17 cells, FIG. 23, without affecting the T_{reg} cells, FIG. 24. Taken together, our data indicated that a combination of Formula (III) and anti-p40 antibody is more effective as compared with single treatment in amelioration of murine colitis.

### Example 4. Study of Formula (III) α4β7 Integrin Small Molecule Inhibitor in Patients with Ulcerative Colitis (UC)

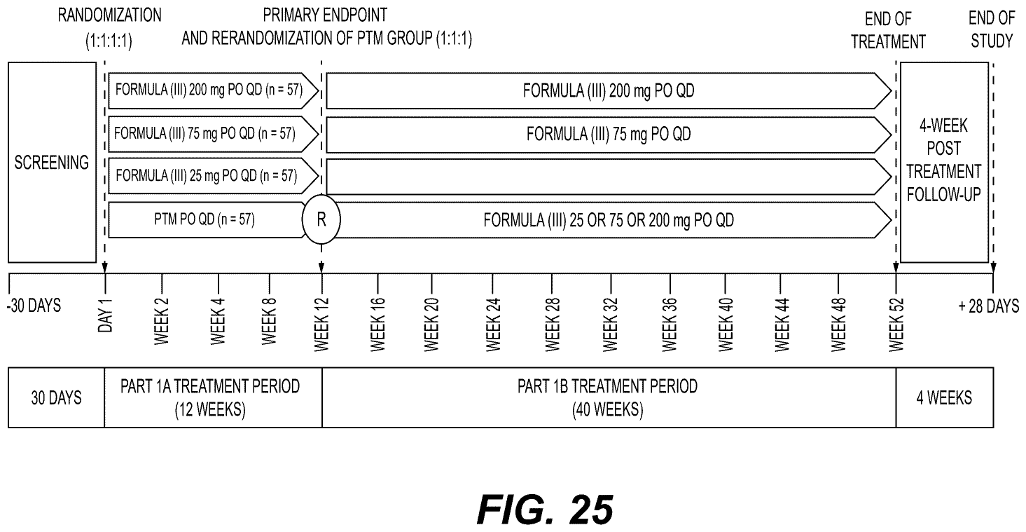

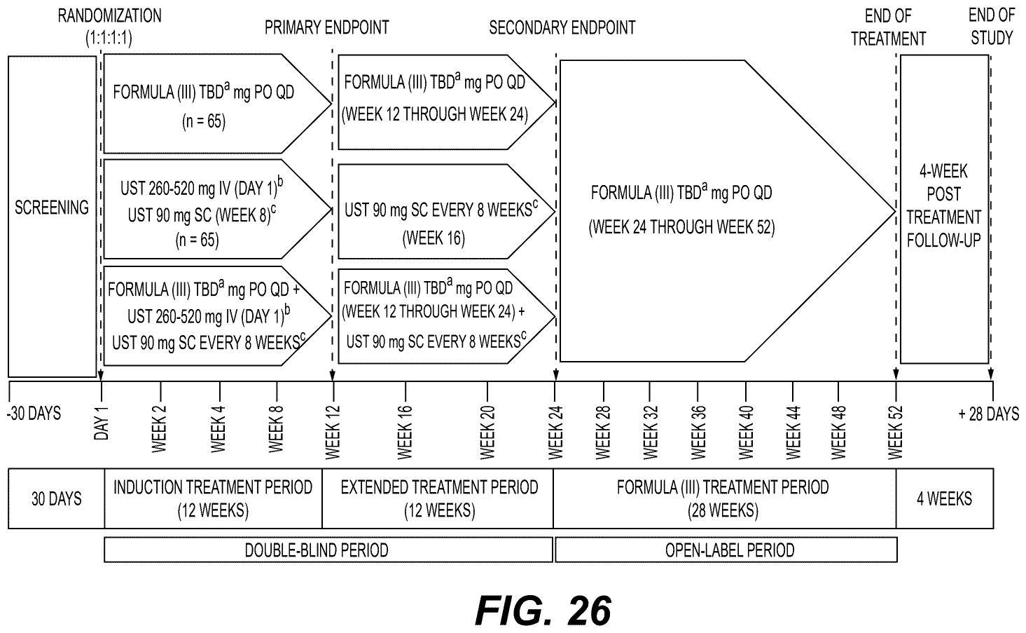

This is a 2-part Phase 2 study to evaluate the efficacy and safety of Formula (III) in monotherapy (Part 1) and subsequently in combination therapy (Part 2) in adult participants with moderately to severely active UC. The schema for Part's 1 and 2 are shown in FIGS. 25 and 26.

### Part 1: Dose-Ranging Study of α4β7 Integrin Small Molecule Inhibitor of Formula (III)

### Part 1:

Dose-ranging study of Formula (III) compound, including the screening period, Part 1A (Day 1 through Week 12), and Part 1B (immediately after Week 12 visit through Week 52).

### Part 2:

Active-controlled, combination study of Formula (III) and ustekinumab, including the screening period, blinded induction treatment (Day 1 through Week 12), blinded extended treatment (immediately after Week 12 visit through Week 24), and open label Formula (III) treatment (immediately after Week 24 visit through Week 52).

### Participant Population

### Number of Participants Planned:

A total of 423 participants is planned for this study. In Part 1, 228 participants who meet all the eligibility criteria at screening will be randomized 1:1:1:1 to receive Formula (III)/25 mg, Formula (III)/75 mg, Formula (III)/200 mg, or PTM. In Part 2, 195 participants who meet all eligibility criteria at screening will be randomized 1:1:1 to receive Formula (III)/monotherapy, ustekinumab monotherapy, or Formula (III)/plus ustekinumab combination therapy (Formula (III)/Part 2 dose to be determined).

### Study Population:

### Inclusion Criteria:

1) Participants are assigned male at birth or nonpregnant, nonlactating participants assigned female at birth, 18 to 75 years of age at randomization.
2) Participants have UC of at least 90-days duration before randomization confirmed by endoscopy and histology at any time in the past.
3) Participants have UC with minimum disease extent of 15 cm from the anal verge.
4) Participants have moderately to severely active UC as determined by endoscopy occurring during screening with a total mMCS of 5 to 9 points, including a centrally read endoscopic subscore of at least 2.
5) Participants have an inadequate response or loss of response or is intolerant to at least 1 of the following conventional UC treatments:
   Corticosteroids
      Active disease despite a history of at least an induction regimen of a dose equivalence to oral prednisone 30 mg daily for 2 weeks; OR
      2 failed attempts to taper corticosteroids below a dose equivalent of 10 mg daily prednisone; OR
      History of corticosteroids intolerance including, but not limited to, Cushing's syndrome, osteopenia/osteoporosis, hyperglycemia, insomnia, serious infections, depressions, allergic reactions, mood disturbances, or any other conditions that contributed to discontinuation of the agent.
   Immunomodulators:
      Active disease despite a history of at least a 12-week regimen of oral azathioprine (≥2 mg/kg/day) or mercaptopurine (6-MP) (≥1 mg/kg/day); OR
      History of intolerance to azathioprine or 6-MP including, but not limited to, serious infections, hepatotoxicity, cytopenia, pancreatitis, thiopurine methyltransferase (TPMT) genetic mutation (any genotype other than *1/*1), allergic reactions, or another condition to discontinuation of the agent.
OR (Participants must meet criteria #5 or #6)
6) Participants have an inadequate response or loss of response or are intolerant to 1 to ≤2 AT mechanisms of action UC:
   TNF-α inhibitor (e.g., infliximab, adalimumab, golimumab, or biosimilars)
   IL-12/23 inhibitor (e.g., ustekinumab) (Part 1 only: No prior exposure in Part 2)
   Sphingosine 1-phosphate receptor modulator (e.g., ozanimod)
   Janus kinase inhibitor (e.g., tofacitinib, filgotinib, upadacitinib)
7) Participants who have a history of UC for 8 or more years must have documentation of a surveillance colonoscopy for screening for dysplasia in the 24 months immediately prior to screening.
8) All participants who have a history of UC for <8 years must be up to date on colon cancer screening prior to randomization per local or national guidelines, as based on age, family history, or increased personal risk of colon cancer.
9) Participants may be receiving concomitant therapy for UC at the time of enrollment as specified below, provided the dose prescribed has been stable as indicated prior to screening endoscopy:
   Participants may be receiving oral 5-aminosalicylate (5-ASA) compounds provided the dose prescribed has been stable for at least 6 weeks immediately prior to screening endoscopy.
   Participants may be on oral corticosteroids at a dose equivalent of prednisone≤20 mg provided the dose has been stable for 2 weeks immediately prior to screening endoscopy.
10) Participants must meet the following TB screening criteria:
   No evidence of active TB, latent TB, or inadequately treated TB as evidenced by symptom evaluation by the investigator and 1 of the following:
   A negative QuantiFERON TB-Gold test or equivalent assay reported by the central laboratory at screening or within 90 days prior to randomization for participants rescreening. OR
   A history of fully treated active or latent TB according to local standard of care. Investigator must verify adequate previous anti-TB treatment and provide documentation; these participants do not require QuantiFERON testing, and eligibility must be approved by the sponsor prior to enrollment in the study; AND
   A chest radiograph (views as per local guidelines with the report or films available for investigator review) taken at screening or within the 4 months prior to randomization without evidence of active or latent TB infection.
11) Participants must have laboratory assessments at screening within the following parameters:
   a. Aspartate aminotransferase (AST) and alanine aminotransferase (ALT)≤2× upper limit of normal (ULN)
   b. Direct bilirubin <1.5×ULN,
   c. Estimated glomerular filtration rate (eGFR)>60 mL/min (using the applicable Chronic Kidney Disease Epidemiology Collaboration [CKD-EPI] equation as outlined below.
      Clinical laboratory evaluations will be performed centrally (except in the event of COVID-19 infection or any natural disasters when a local laboratory for safety laboratory assessments can be used).
      Blood samples for clinical chemistry and hematology assessments will be collected during all study visits, ET, and PTx.
      Blood samples for coagulation assessments may be performed for an AE assessment at any unscheduled visit as determined by the investigator. During the study, for any participant with an initial AST or ALT elevation >3×ULN, at the time of the second confirmatory blood draw, an INR, PT, and total bilirubin must also be drawn.
      In order to allow for assessment of renal function, creatinine and estimated glomerular filtration rate based on serum creatinine (eGFRcr) will be routinely measured.
13) Participants have a negative urine drug screen result. A positive drug screen will exclude participants unless it can be explained by the use of a medication (prescription or nonprescription) that is being used under the direction of a physician. Cocaine use is exclusionary.
14) If assigned female at birth and of childbearing potential, participants must have a negative pregnancy test at screening and agree to use pregnancy testing and contraception.
15) Participants assigned male at birth who engage in heterosexual intercourse must agree to use method(s) of contraception).

### Exclusion Criteria:

Participants who meet any of the following exclusion criteria are not eligible to be enrolled in this study:
1) Have a current diagnosis of Crohn's Disease (“CD”) or clinical findings suggestive of CD, diagnosis of indeterminate colitis due to etiologies such as an enteric pathogen, or lymphocytic or collagenous colitis.
2) Have a current diagnosis of toxic megacolon, symptomatic colonic stricture, acute severe colitis, fulminant colitis, or abdominal abscess at screening or randomization.
3) Have a history of microscopic colitis, ischemic colitis, radiation colitis, or ileoanal pouch anastomosis.
4) Have a history of ileostomy, colostomy, or known fixed symptomatic stenosis of the intestine.
5) Have a history of extensive colonic resection, subtotal or total colectomy or anticipated surgical intervention for UC or another colon disease during the study.
6) Part 1 and Part 2: have any history of exposure to vedolizumab.
7) Part 2 only: have any history of exposure to interleukin 12/23 or 23 inhibitor (eg ustekinumab.
8) Are on antidiarrheal drugs and are not willing to discontinue the medication at least 2 weeks immediately prior to screening endoscopy.
9) Have a history or evidence of incompletely resected colonic mucosal dysplasia or presence of adenomatous colon polyps which were not removed completely prior to randomization.
10) Have a history of fecal microbiota transplant for UC within 24 weeks prior to screening endoscopy.
11) Used IV corticosteroids within 2 weeks prior to screening.
12) Used rectal corticosteroids, rectal mesalamine/rectal 5-ASA (enemas or suppositories) within 2 weeks prior to screening endoscopy.
13) Planned concurrent treatment with immunomodulatory agents (eg, azathioprine, 6MP) after randomization. Participants receiving azathioprine or 6 MP at screening must discontinue treatment with these agents prior to 2 weeks before screening endoscopy.
14) Chronic NSAID use (note: occasional use of NSAIDs for (eg, headache, arthritis, or menstrual cramps) permitted.
15) Have a known history of significant gastric malabsorption which would prevent absorption of the study treatment.
16) Have any history of stroke, seizure disorder, multiple sclerosis, neurodegenerative diseases of brain (such as Parkinson's disease, dementias), or brain tumor.
17) Have a positive progressive multifocal leukoencephalopathy (PML) symptomatic checklist at screening and at randomization prior to the administration of the first dose of study drug.
18) Have evidence of or treatment for Clostridium difficile (C difficile) infection within 60 days prior to randomization, or other intestinal pathogens such as Salmonella, Shigella, Campylobacter jejuni, enterohemorrhagic E. coli or parasitic intestinal infections (such as amebiasis or giardiasis) at screening laboratory examination.
19) Have an active clinically significant extraintestinal infection or any infection requiring hospitalization or treatment with intravenous anti-infective therapy within 8 weeks prior to randomization.
20) Have a chronic infection (infections lasting >3 months) requiring extended therapy: use of any chronic systemic (oral or intravenous) anti-infective therapy including preventive therapy for chronic infections (such as Pneumocystis carinii, cytomegalovirus or herpes zoster) within 6 months prior to screening.
21) Have a positive HIV antibody test.
22) Have a positive HBV surface antigen test. Participants with negative hepatitis B surface antigen (HBsAg) test and positive hepatitis B core antibody (HBcAb) test must have an HBV DNA less than the LLOQ.
23) Have a positive HCV antibody test and HCV RNA greater than the LLOQ. A positive HCV antibody test is defined as a participants with positive HCV antibody at screening must have further testing for HCV RNA. Participants with positive HCV antibody at screening must have further testing for HCV RNA. Participants with HCV RNA greater than or equal to the LLOQ will not be eligible for the study. Participants with positive HCV antibody but HCV RNA less than the LLOQ are eligible.
24) Has or has ever had a nontuberculous mycobacterial infection or serious opportunistic infection (eg, cytomegalovirus colitis, Pneumocystis carinii, aspergillosis).
25) Are unable to take oral medication or are dependent on total parenteral nutrition.
26) Have current malignancy or a history of malignancy within the past 5 years prior to screening except for adequately treated nonmelanoma skin cancer or cervical carcinoma in situ that has not recurred for at least 1 year prior to randomization.
27) Have a history of lymphoproliferative disorder, lymphoma, leukemia, myeloproliferative disorder, or multiple myeloma.
28) Have any progressive chronic medical condition, including, but not limited to, cardiac, pulmonary, renal, hepatic (e.g., cirrhosis, primary sclerosing cholangitis), or psychiatric problem (including alcohol or drug abuse) that, in the opinion of the investigator or sponsor, would make the participant unsuitable for the study or would prevent compliance with the study protocol.
29) Have a transplanted organ except for a corneal transplant.
30) Have had any major surgery or trauma (as determined by the principal investigator) within 8 weeks prior to randomization.
31) Are likely to require any type of major surgery during the study.
32) Have a history of symptomatic herpes zoster within 16 weeks of randomization or any history of disseminated herpes simplex, disseminated herpes zoster, ophthalmic zoster, or varicella zoster virus central nervous system infections.
33) Received a live-attenuated vaccine within 4 weeks of randomization.
34) Are participants assigned female at birth who may wish to become pregnant and/or plan to undergo egg donation or egg harvesting for the purpose of current or future fertilization during the study and up to 14 days (or by the institutional guideline as indicated, whichever is longer) after the last dose of study drug administered during Part 1 of this clinical study, or until 15 weeks after the last administered injection of ustekinumab/ustekinumab PTM or 14 days after the last dose of Formula (III)/Formula (III) PTM, whichever date comes later during Part 2 of this clinical study.
35) Are participants assigned male at birth who have a female partner of childbearing age and unwilling to refrain from sperm donation for at least 14 days (or by the institutional guideline as indicated, whichever is longer) after last dose of study drug administered during Part 1 of this clinical study or until 15 weeks after the last administered injection of ustekinumab/ustekinumab PTM or 14 days after the last dose of Formula (III)/Formula (III) PTM, whichever date comes later during Part 2 of this clinical study.
36) Have any known condition or contraindication as addressed in the local labeling for ustekinumab that would preclude the participant from participating in Part 2 of this study.
37) Requirement for ongoing therapy with or use of any prohibited medication as specified in the protocol (See Tables 19 and 20).

TABLE 19
Prior and Concomitant Medications That Are Prohibited for Defined Period
Before Enrollment Because of Impact on Efficacy Assessments or Participant Safety

| Drug Class | Agents Disallowed | Prohibited Period |
|---|---|---|
| TNF-α antagonist | Infliximab, adalimumab, | 8 weeks prior to screening |
| | golimumab, | endoscopy or an undetectable |
| | or biosimilar agent | serum level on a commercially |
| | | available assay through the EOT |
| Integrin antagonist | vedolizumab, natalizumab | Any time prior to study |
| | carotegrast | enrollment through EOT |
| | or any investigational | |
| | anti-integrin | |
| Interleukin antagonist | Ustekinumab and other | 15 weeks prior to screening |
| | approved IL antagonists^{c} | endoscopy or an undetectable |
| | | serum level on a commercially |
| | | available assay through the EOT |
| JAK inhibitors | tofacitinib, baricitinib, | 4 weeks prior to screening |
| | filgotinib, | endoscopy through the EOT |
| | upadacitinib | |
| Sphingosine 1- | etrasimod, ozanimod, | 5 half-lives prior to screening |
| phosphate receptor | investigational agents | endoscopy (8 weeks for |
| modulators | | ozanimod and 1 week for |
| | | etrasimod) through the EOT |
| Rectal compounds | rectal mesalamine, rectal | 2 weeks prior to screening |
| | corticosteroids | endoscopy through the EOT |
| Antidiarrheal agents | loperamide | 2 weeks prior to screening |
| | | endoscopy through the EOT |
| Other (nonbiologic) | azathioprine, 6-MP, | 2 weeks prior to screening |
| | thalidomide, tacrolimus, | endoscopy through the EOT |
| | sirolimus, methotrexate | |
| Other (nonbiologic) | leflunomide | Exclude for 5- half-lives or |
| | | 10 weeks prior to screening |
| | | endoscopy through EOT |
| Other | All immunosuppressants | 5-half-lives prior to screening |
| (immuno- | disallowed except | endoscopy |
| suppressants) | OCS at ≤20 mg | |
| Investigational agents | Any investigational | 12 weeks prior to screening |
| | biologic agent and | endoscopy through the EOT |
| | nonbiologic agent | (or at least 5 half-lives) |
| Lymphocyte- | alemtuzumab, | Any time before study |
| depleting therapies | cyclophosphamide, | enrollment through the EOT |
| | mycophenolate, cyclosporin, | |
| | total lymphoid irradiation, | |
| | rituximab, antithymocytic | |
| | globulin, and any other | |
| | lymphocyte-depleting therapy | |
| Antibiotics if used for | ciprofloxacin, metronidazole, | 2 weeks prior to screening |
| primary | rifaximin | endoscopy through the EOT |
| treatment of UC | | |

EOT = end of treatment;
IL = interleukin;
JAK = Janus kinase;
OCS = oral corticosteroid;
TNF-α = tumor necrosis factor-alpha;
UC = ulcerative colitis
d Not all of these example medications may be approved in each of the countries where the study is being conducted; please refer to local product information.
e Table is not all-inclusive. Topical formulation of above medications may be allowed. Consult medical monitor (or designee) as needed.
f Interleukin antagonist is allowed in Part 1 only and not allowed in Part 2 of the study.

If other prohibited concomitant medications, as presented in Table 19 and 20 are taken by a participant prior to study participation or initiated during the study, investigators must contact the medical monitor.

TABLE 20
Other Prohibited Concomitant Medications

| Drug Class | Agents Disallowed | Prohibited Period |
|---|---|---|
| Chronic NSAIDs^{a} | Ibuprofen, naproxen, | 2 weeks prior to screening |
| | diclofenac, indomethacin | endoscopy through end |
| | | of treatment |
| Other biologics^{b} | Antibody-based or other | Requires Gilead Medical |
| | systemic biologics, e.g., | Monitor consultation |
| | denosumab, trastuzumab | |
| Other biologics^{c} | Fecal microbiota transplant | Fecal microbiota |
| | | transplant |
| | | for ulcerative colitis |
| | | within 24 weeks prior to |
| | | screening endoscopy |
| Certain | Diphenoxylate/atropine, | 2 weeks prior to screening |
| antidiarrheal/ | metoclopramide, cisapride, | endoscopy through end |
| antimotility agents | bismuth subsalicylate | of treatment |
| Vaccine | BCG | Should not have received |
| | | BCG vaccine 1 year before |
| | | ustekinumab treatment |
| | | and 1 year after stopping |
| | | ustekinumab |

BCG = Bacillus Calmette-Guérin;
NSAID = nonsteroidal anti-inflammatory drug;
UC = ulcerative colitis
g Occasional use of NSAIDs for transient symptoms and daily use of aspirin up to 162.5 mg for the purpose of cardiovascular prophylaxis are permitted. List of NSAID medications is not all-inclusive.
h Other biologics may be allowed with the approval of the medical monitor.
i Only if fecal microbiota is used for UC treatment.

### Treatment Arms

Screening Period: Days −30 to −1

### Part 1A (Day 1 Through Week 12):

Blinded Treatment: Day 1 through Week 12 (Part 1A)
   At baseline/randomization (Day 1), approximately 228 participants who meet protocol eligibility criteria will be randomized in a 1:1:1:1 ratio to 1 of 4 treatment groups as follows:
Treatment Group 1 (n=57): Formula (III) 200 mg orally once daily
Treatment Group 2 (n=57): Formula (III) 75 mg orally once daily
Treatment Group 3 (n=57): Formula (III) 25 mg orally once daily
Treatment Group 4 (n=57): Placebo orally once daily

### Week 12 Primary Analysis

At completion of Week 12 for all participants in Part 1A, a primary analysis of efficacy and safety will be performed.

Part 1B Treat-Through Groups (Immediately after Week 12 Visit Through Week 52):
Participants in the Formula (III) treatment groups who complete the 12-week Part 1A treatment period will be allowed to continue into Part 1B as an extension period and remain on the same dose of Formula (III) treatment as assigned at randomization. Consideration should be given to discontinuing study drug for participants who do not show clinical improvement per the Investigator's judgement by Week 24. Consideration should also be giving to discontinuing study drug for participants who meet disease worsening criteria (see below) between Week 12 and Week 52. Participants who permanently discontinue study drug during the Part 1B will be withdrawn from the study, complete early termination (“ET”) visit, and be asked to return for a follow up approximately 4 weeks after their last study drug dose to assess safety.
   Part 1B Rerandomization Group (Immediately after Week 12 Visit Through Week 52):

Participants in the placebo group who complete the 12-week Part 1A treatment period will be eligible to be rerandomized in a double-blind manner at Week 12 to 1 of the 3 active treatment groups.

Participants in Part 1 may receive a maximum of 52 weeks of treatment with study drug Formula (III) (12 weeks during Part 1A and 40 weeks during Part 1B).

The decision to initiate Part 2 of the study, including dose(s) selection of Formula (III) for Part 2 in the blinded and open label treatment periods, will be based on the sponsor review of efficacy and safety data from Part 1A (Week 12 primary analysis).

Participants will not roll over from Part 1 into Part 2 of the study.

### Part 2:

Active-controlled, combination study of Formula (III) and ustekinumab, including the screening period, blinded induction treatment (Day 1 through Week 12), blinded extended treatment (immediately after Week 12 visit through Week 24), and open label Formula (III) treatment (immediately after Week 24 visit through Week 52).
Screening Period: Days −30 to −1

### Blinded Induction Treatment (Day 1 Through Week 12):

At baseline/randomization (Day 1), approximately 195 participants who meet protocol eligibility criteria will be randomized in a 1:1:1 ratio to 1 of 3 active treatment groups as follows:
Treatment Group 1 (n=65): Formula (III) monotherapy (either 25 mg. 75 mg, or 100 mg dosed orally once daily)
Treatment Group 2 (n=65): ustekinumab monotherapy (initial dose determined by body weight: 260 to 520 mg intravenously [IV]; subsequent dose: 90 mg subcutaneously (“SC”) 8 weeks after the initial IV dose, then every 8 weeks thereafter)
Treatment Group 3 (n=65): Formula (III) and ustekinumab combination therapy (Formula (III) either 25 mg. 75 mg, or 100 mg dosed orally once daily; ustekinumab initial dose will be determined by body weight: 260 to 520 mg IV; ustekinumab subsequent dose: 90 mg SC 8 weeks after the initial IV dose, then every 8 weeks thereafter)

### Week 12 Primary Analysis

At completion of Week 12 for all participants in Blinded Induction Treatment, a primary analysis of efficacy and safety will be performed.

Blinded Extended Treatment (Immediately after Week 12 Visit Through Week 24):

Participants who do not meet disease worsening criteria shall continue the same treatment assigned Day 1 through Week 12 for another 12 weeks (Blinded Extended Treatment). Consideration should be given to discontinuing study drug for participants who do not show clinical improvement per the investigator's judgement, or who meet disease worsening criteria.

Open Label Study: Formula (III) Treatment with Formula (III) Immediately after Week 24 Visit Through Week 52):

Participants who complete 24 weeks treatment will be eligible for 28 weeks of open-label (“OL”) Formula (III) treatment. Participants will be closely monitoring for improvement or disease worsening throughout 28 weeks of Formula (III) treatment. Consideration will also be giving to discontinuing study drug for Participants who do not show clinical improvement per the investigator's judgement between Week 24 and Week 52. Participants who permanently discontinue study drug during the OL Formula (III) treatment period shall be withdrawn from the study and complete the ET visit. For participants who withdraw or discontinue from the study prior to Week 32, the follow-up visit should be 19 weeks after the last injection of ustekinumab (Uste)/ustekinumab PTM or 28±2 days after the last administered dose of Formula (III)/Formula (III) placebo-to match (“PTM”), based on whichever date comes later. For participants who withdraw or discontinue from the study at or after the start of Week 32, the follow-up visit should be 28±2 days after the last administered dose of Formula (III)/Formula (III) PTM.

### Dosage and Administration

In Part 1 of the study, study drug will consist of Formula (III) 100-mg, 75-mg, and 25-mg tablets, and Formula (III) PTM tablets for oral administration on an empty stomach (see dietary restrictions below).

In Part 2 of the study, study drug will consist of Formula (III) Formula (III) tablets and Formula (III) PTM tablets for oral administration (dose to be determined) on an empty stomach (see dietary restrictions below). Part 2 of the study will also utilize ustekinumab (260-520 mg) and ustekinumab PTM for administration by IV infusion as the first dose, as well as ustekinumab (90 mg) and ustekinumab PTM for subsequent 8 weekly administrations by SC injection.

### Part 1A Treatment Period

The tablets will be administered to each treatment group in the Part 1A treatment period, Day 1 through Week 12, as follows:
Treatment Group 1 (Formula (III) 200 mg): 1 Formula (III) 100-mg tablet, 1 Formula (III) Formula (III) 75-mg tablet, and 1 Formula (III) Formula (III) 25-mg tablet, orally once daily
Treatment Group 2 (Formula (III) 75 mg): 1 Formula (III) 75-mg tablet, 1 placebo-to-match (PTM) 100-mg tablet, and 1 PTM 25-mg tablet, orally once daily
Treatment Group 3 (Formula (III) Formula (III) 25 mg): 1 Formula (III) 25-mg tablet, 1 PTM 100-mg tablet, and 1 PTM 75-mg tablet, orally once daily
Treatment Group 4 (Placebo): 1 PTM 100-mg tablet, 1 PTM 75-mg tablet, and 1 PTM 25 mg tablet, orally once daily
In all cases, participants will take 3 tablets in total per day.

### Part 1B Treatment Period

The tablets administered to each treatment group in the Part 1B treatment period, Week 12 through Week 52, are as follows:
Treatment Group 1 (Formula (III) 200 mg): 1 Formula (III) 100-mg tablet, 1 Formula (III) 75-mg tablet, and 1 Formula (III) 25-mg tablet, orally once daily
Treatment Group 2 (Formula (III) 75 mg): 1 Formula (III) 75-mg tablet, 1 PTM 100-mg tablet, and 1 PTM 25-mg tablet, orally once daily.
Treatment Group 3 (Formula (III) 25 mg): 1 Formula (III) 25-mg tablet, 1 PTM 100-mg tablet, and 1 PTM 75-mg tablet, orally once daily
In all cases, participants will take 3 tablets in total per day.

### Part 2 (Blinded Induction) Treatment Period

The study drugs/comparators will be administered to each treatment group in the Induction Treatment period, Day 1 through Week 12, as follows:
Treatment Group 1 (Formula (III) monotherapy): 1 or 2 Formula (III) tablets (dose to be determined) orally once daily; ustekinumab PTM IV infusion on Day 1; ustekinumab PTM SC injection at Week 8
Treatment Group 2 (ustekinumab monotherapy): 1 or 2 Formula (III) PTM tablets (dose to be determined) orally once daily; ustekinumab IV infusion on Day 1 (260-520 mg); ustekinumab SC injection at Week 8 (90 mg)
Treatment Group 3 (Formula (III) and ustekinumab combination therapy): 1 or 2 Formula (III) tablets (dose to be determined) orally once daily; ustekinumab IV infusion on Day 1 (260-520 mg); ustekinumab SC injection at Week 8 (90 mg)
In all cases, participants will take Formula (III) or Formula (III) PTM tablets (dose to be determined), have an IV infusion at Day 1, and a SC injection at Week 8.

### Part 2 (Blinded Extended Treatment) Treatment Period

The study drugs/comparators will be administered to each treatment group in the Induction Treatment period, Day 1 through Week 12, as follows:
Treatment Group 1 (Formula (III) monotherapy): 1 or 2 Formula (III) tablets (dose to be determined) orally once daily; ustekinumab PTM SC injection every 8 weeks (last and only dose will be Week 16)
Treatment Group 2 (ustekinumab monotherapy): 1 or 2 Formula (III) PTM tablets (dose to be determined) orally once daily; ustekinumab SC injection (90 mg) every 8 weeks (last and only dose will be Week 16)
Treatment Group 3 (Formula (III) and ustekinumab combination therapy): 1 or 2 Formula (III) tablets (dose to be determined) orally once daily; ustekinumab SC injection (90 mg) every 8 weeks (last and only dose will be Week 16)
   In all cases of the Part 2 Extended Treatment Period, participants will take Formula (III) tablets or Formula (III) tablets PTM (dose to be determined) and have an ustekinumab or ustekinumab PTM SC injection at Week 16.

### Dietary Restrictions

Formula (III) should be administered without food on an empty stomach only with water, and no other medications or supplements should be taken with study drug; food can be consumed 4 hours before and 2 hours after the administration of Formula (III). Participants will be required to refrain from the consumption of iron and calcium at least 3 hours after the study drug. Participants should be instructed to administer study drug at or around the same time each day on an empty stomach. If one or more study drug tablets are not taken at the same time on a given day, participants should be instructed to take the missed tablets for that day as soon as possible during the same day. If one or more study drug tablets are not taken during the same day, participants should be instructed to not take any missed tablets on subsequent days so as to never exceed the regular dosage. Participants should note missed tablets in the drug diary. Participant's unused study drug should be returned to the site to account for study drug compliance.

### Open Label Formula (III) Treatment Period (Week 24 Through Week 52)

Treatment Group 1 (Formula (III) monotherapy): 1 or 2 Formula (III) tablets (dose to be determined) orally once daily.

### Statistical Methods:

### Primary Analysis (Week 12)

In Part 1 of the study, a Week 12 primary analysis will be conducted after all randomized participants have completed 12 weeks of blinded treatment in Part 1A or discontinued from the study, and associated efficacy and safety assessments have been completed. Part 1A of the study will be unblinded to Gilead personnel involved in the primary analysis. Week 12 efficacy and safety analyses, including the primary analysis, will be performed. The purpose of these analyses is to inform the initiation of Part 2 of the study.

In Part 2 of the study, a Week 12 primary analysis will be conducted after all randomized participants have completed 12 weeks of blinded treatment in Part 2A or discontinued from the study, and associated efficacy and safety assessments have been completed. Part 2A of the study will be unblinded to Gilead personnel involved in the primary analysis. Week 12 efficacy and safety analyses, including the primary analysis, will be performed.

The Cochran Mantel Haenszel approach adjusting for stratification factors will be used for hypothesis testing of the primary endpoint. Nominal P values unadjusted for multiplicity will be calculated. For the evaluation of clinical response at Week 12, mMCS subscores (i.e., stool frequency, rectal bleeding, and endoscopic findings (by central readers) at screening will be used as the baseline value.

### Final Analysis

A final analysis will be performed at the end of study for Part 1 and Part 2, respectively. For each part, the final analysis will be conducted after all participants have completed the respective part of the study, outstanding data queries have been resolved or adjudicated as unresolvable, and the data have been cleaned and finalized.

For Part 1, descriptive statistics for secondary endpoints at Week 52 will be provided within each Formula (III) group. For each binary endpoint, number and proportion of participants achieving respective criteria at Week 52 will be provided. No statistical comparisons will be performed.

For Part 2, the estimand for each secondary binary endpoint at Week 52 is defined with similar attributes as the primary estimand for Part 2, except that treatment groups, variable (endpoint) and population-level summary are treatment- and endpoint-specific.

### Safety

The safety analysis for Part 2 will be performed separately.

### Sample Size

### Part 1

A sample size of 57 participants per group in all active Formula (III) treatment groups and the placebo group has at least 80% power to detect an absolute difference of 27.5% in clinical response between an individual treatment group and placebo at Week 12. Power was calculated using Fisher's Exact Test at a 2-sided significance level of 0.05. The placebo response rate is assumed to be 30%, and the response rate in an active Formula (III) treatment group is assumed to be 57.5%.

The placebo response rate of 30% was estimated based on placebo response rates observed in several Phase 3 studies, assuming 50% of participants are advanced therapy naive, and 50% are advanced therapy experienced. The Phase 3 studies used in the calculation are: ACCOMPLISH (upadacitinib), ACHIEVE (upadacitinib), GEMINI 1 (vedolizumab), OCTAVE 2 (tofacitinib), SELECTION (filgotinib), UNIFI (ustekinumab), and OCTAVE 1 (tofacitinib).

The treatment effect of 27.5% was estimated based on the difference in proportion of participants achieving clinical response at Week 6 between vedolizumab and placebo groups in the GEMINI 1 study, assuming 50% of participants are advanced therapy naive, and 50% are advanced therapy experienced. Since the primary endpoint is clinical response at Week 12 in this study, a 25% increase in treatment effect size from GEMINI 1 was assumed.

When calculating response rates in previous Phase 3 studies, participants who prematurely discontinued from the study before primary endpoint assessment were considered non-responders. About 8% of participants in this study are assumed to prematurely discontinue before Week 12 and will be considered non-responders for the primary endpoint, which is within the range of study discontinuation rates observed in these previous Phase 3 studies.

### Part 2

A sample size of 65 participants per group in the Formula (III) monotherapy, ustekinumab monotherapy, and Formula (III) and ustekinumab combination therapy groups has at least 80% power to detect an absolute difference of 20% at a 1-sided significance level of 0.10 in clinical response between the Formula (III) and ustekinumab combination therapy group and an individual active control (monotherapy) group at Week 12. Power was calculated using Fisher's Exact Test at a 1-sided significance level of 0.10. The response rate in an active control treatment group is assumed to be 55%.

### Objectives and Endpoints:

### Part 1 Primary Objectives:

To assess the efficacy of Formula (III) (Example Compund 1), compared with placebo control, in achieving clinical response at Week 12.

### Part 1 Primary Endpoints:

Clinical response at Week 12. Clinical response is defined as a decrease from baseline in the mMCS of ≥2 points and at least a 30% reduction from baseline, and a decrease in rectal bleeding subscore of ≥1 from baseline or an absolute rectal bleeding subscore of 0 or 1.

### Part 1 Secondary Objectives:

To evaluate the safety and tolerability of Formula (III), up to Week 52.
To assess the efficacy of Formula (III), compared with placebo control, in achieving clinical remission at Week 12.
To assess the efficacy of Formula (III), compared with placebo control, in achieving clinical remission at Week 52.
To assess the efficacy of Formula (III), compared with placebo control, in achieving histologic-endoscopic mucosal improvement at Week 12.
To assess the efficacy of Formula (III), compared with placebo control, in achieving mucosal healing at Week 12.
To assess the efficacy of Formula (III), compared with placebo control, in achieving endoscopic improvement at Week 12.

### Part 1 Endpoints:

Incidence of TEAEs, SAEs, or deaths, and treatment-emergent laboratory abnormalities.
Clinical remission at Week 12. Clinical remission is defined as an mMCS of ≤2, including a stool frequency subscore (SFS)≤1 and not greater than baseline, rectal bleeding subscore of 0, and centrally read endoscopic subscore≤1 (score of 1 modified to exclude friability).
Clinical remission at Week 52. Clinical remission is as defined above.
Histologic-endoscopic mucosal improvement at Week 12. Histologic-endoscopic mucosal improvement is defined as a Geboes score≤3.1 and endoscopic subscore of ≤1.
Mucosal healing at Week 12. Mucosal healing is defined as a Geboes score≤2B.1 and endoscopic subscore≤1.
Endoscopic improvement at Week 12. Endoscopic improvement is defined as an endoscopic subscore of ≤1.

### Part 2 Primary Objectives:

To assess the efficacy of combination therapy with Formula (III) and ustekinumab, compared with Formula (III) and ustekinumab monotherapies, in achieving clinical response at Week 12.

### Part 2 Primary Endpoints:

Clinical response at Week 12: Clinical response is defined as a decrease from baseline in the mMCS of ≥2 points and at least a 30% reduction from baseline, and a decrease in rectal bleeding subscore of ≥1 from baseline or an absolute rectal bleeding subscore of 0 or 1.

### Part 2 Secondary Objectives:

To assess the efficacy of combination therapy with Formula (III) and ustekinumab, compared with Formula (III) and ustekinumab monotherapies, in achieving clinical response at Week 24.
To evaluate the safety and tolerability of the combination of Formula (III) and ustekinumab up to Week 24.
To evaluate the safety and tolerability of Formula (III) up to Week 52.
To assess the efficacy of combination therapy with Formula (III) and ustekinumab, compared with Formula (III) and ustekinumab monotherapies, in achieving clinical remission at Week 12.
To assess the efficacy of combination therapy with Formula (III) and ustekinumab, compared with Formula (III) and ustekinumab monotherapies, in achieving mucosal healing at Week 12.
To assess the efficacy of combination therapy with Formula (III) and ustekinumab, compared with Formula (III) and ustekinumab monotherapies, in achieving histologic-endoscopic mucosal improvement at Week 24.
To assess the efficacy of combination therapy with Formula (III) and ustekinumab, compared with Formula (III) and ustekinumab monotherapies, in achieving endoscopic improvement at Week 12.
To assess the efficacy of combination therapy with Formula (III) and ustekinumab, compared with Formula (III) and ustekinumab monotherapies, in achieving endoscopic improvement at Week 24.

### Part 2 Endpoints:

Clinical response at Week 24.
Incidence of TEAEs, SAEs, or deaths, and treatment-emergent laboratory abnormalities
Clinical remission at Week 12. Clinical remission is defined as an mMCS of ≤2, including a stool frequency subscore (SFS)≤1 and not greater than baseline, rectal bleeding subscore of 0, and centrally read endoscopic subscore≤1 (score of 1 modified to exclude friability).
Clinical remission at Week 24. Clinical remission is as defined above.
Partial modified Mayo score remission at Week 52. Partial modified Mayo score remission is defined as an SFS of ≤1 and rectal bleeding subscore of 0.
Histologic-endoscopic mucosal improvement at Week 12. Histologic-endoscopic mucosal improvement is defined as a Geboes score≤3.1 and endoscopic subscore of ≤1.
Histologic-endoscopic mucosal improvement at Week 24. Histologic-endoscopic mucosal improvement is as defined above.
Mucosal healing at Week 12. Mucosal healing is defined as a Geboes score≤2B.1 and endoscopic subscore≤1.
Mucosal healing at Week 24. Mucosal healing is as defined above.
Endoscopic improvement at Week 12. Endoscopic improvement is defined as an endoscopic subscore of ≤1.

### Example 5. Study of Formula (III) α4β7 Integrin Small Molecule Inhibitor Mono Therapy in Patients with Ulcerative Colitis (UC)

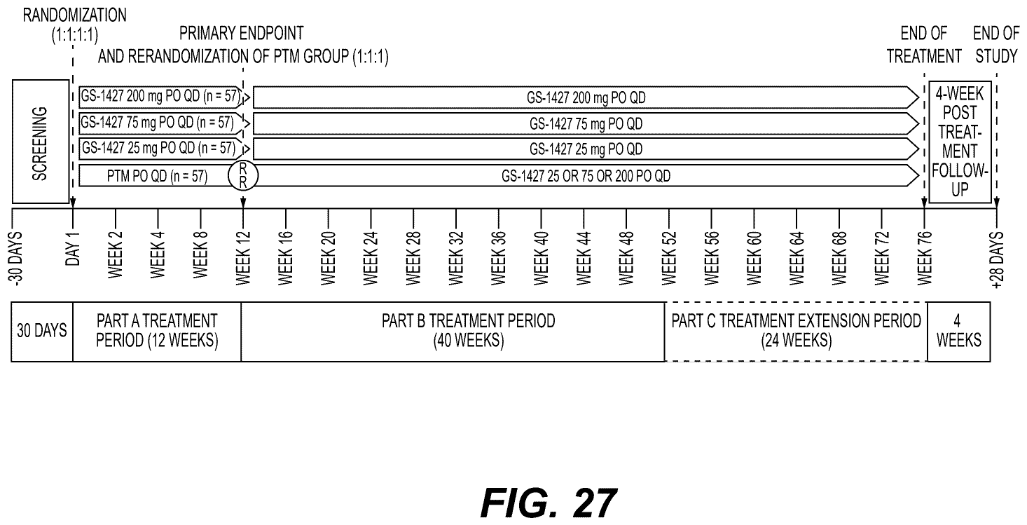

A Phase 2 study to evaluate the efficacy and safety of Formula (III) in adult participants with moderately to severely active UC will be conducted. The schema for Part 1 is shown in FIG. 27.

### Participant Population

### Number of Participants Planned:

A total of 423 participants is planned for this study. In Part 1, 228 participants who meet all the eligibility criteria at screening will be randomized 1:1:1:1 to receive Formula (III)/25 mg, Formula (III)/75 mg, Formula (III)/200 mg, or PTM.

### Study Population:

### Inclusion Criteria:

1) Participants have UC of at least 90-days duration before randomization, with diagnosis confirmed by endoscopy and histology at any time prior.
2) Participants have UC with minimum disease extent of 15 cm from the anal verge.
3) Participants have moderately to severely active UC as determined by endoscopy occurring during screening with a total mMCS of 5 to 9 points, including a centrally read endoscopic subscore of at least 2.
4) Participants have an inadequate response or loss of response or is intolerant to at least 1 of the following conventional UC treatments:
   Corticosteroids
      Active disease despite a history of at least an induction regimen of a dose equivalence to oral prednisone 30 mg daily for 2 weeks; OR
      2 failed attempts to taper corticosteroids below a dose equivalent of 10 mg daily prednisone; OR
      History of corticosteroids intolerance including, but not limited to, Cushing's syndrome, osteopenia/osteoporosis, hyperglycemia, insomnia, serious infections, depressions, allergic reactions, mood disturbances, or any other conditions that contributed to discontinuation of the agent.
   Immunomodulators:
      Active disease despite a history of at least a 12-week regimen of oral azathioprine (≥2 mg/kg/day) or mercaptopurine (6-MP) (≥1 mg/kg/day); OR
      History of intolerance to azathioprine or 6-MP including, but not limited to, serious infections, hepatotoxicity, cytopenia, pancreatitis, thiopurine methyltransferase (TPMT) genetic mutation (any genotype other than *1/*1), allergic reactions, or another condition to discontinuation of the agent.
   Advance therapy: have an inadequate response or loss of response or are intolerant to an advanced therapy (AT) for the treatment of UC:
      TNF-α inhibitor (e.g., infliximab, adalimumab, golimumab, or biosimilars)
      IL-12/23 inhibitor (e.g., ustekinumab)
      Sphingosine 1-phosphate receptor modulator (e.g., ozanimod)
      Janus kinase inhibitor (e.g., tofacitinib, filgotinib, upadacitinib)
5) Participants have an inadequate response or loss of response or are intolerant to <3 AT mechanisms of action for UC (use of 2 or more AT with the same mechanism of action, e.g., 2 TNF-α inhibitors, counts as 1 mechanism of action).
6) All participants who have a history of UC for <8 years must be up to date on colon cancer screening prior to randomization per local or national guidelines, as based on age, family history, or increased personal risk of colon cancer.
7) Optional: Screening laboratory evaluations and 12-lead electrocardiogram (ECG) evaluations must be without clinically significant abnormalities as assessed by the investigator.
8) Optional: Have liver biometric tests such as alanine aminotransferase (ALT), aspartate aminotransferase (AST), alkaline phosphatase, and total bilirubin below the upper limit of normal at screening.
9) Optional: No evidence of active or latent TB, which should be supported by a negative interferon gamma release assay (IGRA) test performed by the laboratory at screening.
10) Optional: Must be willing and able to comply with all study requirements.
11) Optional: Must, in the opinion of the investigator, be in good health based upon medical history and physical examination, including vital signs.

### Exclusion Criteria:

Participants who meet any of the following exclusion criteria are not eligible to be enrolled in this study:
1) Have a current diagnosis of CD or clinical findings suggestive of CD, diagnosis of indeterminate colitis due to etiologies such as an enteric pathogen, or lymphocytic or collagenous colitis.
2) Have a current diagnosis of toxic megacolon, symptomatic colonic stricture, acute severe colitis, fulminant colitis, or abdominal abscess at screening or randomization.
3) Part 1 and Part 2: Have any history of exposure to vedolizumab or other integrin antagonists.
4) Have a history of stroke, seizure disorder, multiple sclerosis, neurodegenerative disease of brain (such as Parkinson's disease, dementias), or brain tumor.
5) Have a positive progressive multifocal leukoencephalopathy subjective checklist at screening or at randomization prior to the administration of the first dose of the study drug.
6) Optional: Have any history of exposure to vedolizumab.
7) Optional: Are on antidiarrheal drugs and are not willing to discontinue the medication at least 2 weeks immediately prior to screening endoscopy.
8) Optional: Have a history or evidence of incompletely resected colonic mucosal dysplasia or presence of adenomatous colon polyps which were not removed completely prior to randomization.
9) Optional: Have a history of fecal microbiota transplant for UC within 24 weeks prior to screening endoscopy.
10) Optional: Used IV corticosteroids within 2 weeks prior to screening.
11) Optional: Used rectal corticosteroids, rectal mesalamine/rectal 5-ASA (enemas or suppositories) within 2 weeks prior to screening endoscopy.
12) Optional: Planned concurrent treatment with immunomodulatory agents (eg, azathioprine, 6MP) after randomization. Participants receiving azathioprine or 6 MP at screening must discontinue treatment with these agents prior to 2 weeks before screening endoscopy.
13) Optional: Chronic NSAID use (note: occasional use of NSAIDs for (eg, headache, arthritis, or menstrual cramps) permitted.
14) Optional: Have a known history of significant gastric malabsorption which would prevent absorption of the study treatment.
15) Optional: Have any history of stroke, seizure disorder, multiple sclerosis, neurodegenerative diseases of brain (such as Parkinson's disease, dementias), or brain tumor.
16) Optional: Have a positive progressive multifocal leukoencephalopathy (PML) symptomatic checklist at screening and at randomization prior to the administration of the first dose of study drug.
17) Optional: Have evidence of or treatment for Clostridium difficile (C difficile) infection within 60 days prior to randomization, or other intestinal pathogens such as Salmonella, Shigella, Campylobacter jejuni, enterohemorrhagic E. coli or parasitic intestinal infections (such as amebiasis or giardiasis) at screening laboratory examination.
18) Optional: Have an active clinically significant extraintestinal infection or any infection requiring hospitalization or treatment with intravenous anti-infective therapy within 8 weeks prior to randomization.
19) Optional: Have a chronic infection (infections lasting >3 months) requiring extended therapy: use of any chronic systemic (oral or intravenous) anti-infective therapy including preventive therapy for chronic infections (such as Pneumocystis carinii, cytomegalovirus or herpes zoster) within 6 months prior to screening.
20) Optional: Have a positive HIV antibody test.
21) Optional: Have a positive HBV surface antigen test. Participants with negative hepatitis B surface antigen (HBsAg) test and positive hepatitis B core antibody (HBcAb) test must have an HBV DNA less than the LLOQ.
22) Optional: Have a positive HCV antibody test and HCV RNA greater than the LLOQ. A positive HCV antibody test is defined as a participant with positive HCV antibody at screening must have further testing for HCV RNA. Participants with positive HCV antibody at screening must have further testing for HCV RNA. Participants with HCV RNA greater than or equal to the LLOQ will not be eligible for the study. Participants with positive HCV antibody but HCV RNA less than the LLOQ are eligible.
23) Optional: Has or has ever had a nontuberculous mycobacterial infection or serious opportunistic infection (eg, cytomegalovirus colitis, Pneumocystis carinii, aspergillosis).
24) Optional: Are unable to take oral medication or are dependent on total parenteral nutrition.
25) Optional: Have current malignancy or a history of malignancy within the past 5 years prior to screening except for adequately treated nonmelanoma skin cancer or cervical carcinoma in situ that has not recurred for at least 1 year prior to randomization.
26) Optional: Have a history of lymphoproliferative disorder, lymphoma, leukemia, myeloproliferative disorder, or multiple myeloma.
27) Optional: Have any progressive chronic medical condition, including, but not limited to, cardiac, pulmonary, renal, hepatic (e.g., cirrhosis, primary sclerosing cholangitis), or psychiatric problem (including alcohol or drug abuse) that, in the opinion of the investigator or sponsor, would make the participant unsuitable for the study or would prevent compliance with the study protocol.
28) Optional: Have a transplanted organ except for a corneal transplant.
29) Optional: Have had any major surgery or trauma (as determined by the principal investigator) within 8 weeks prior to randomization.
30) Optional: Are likely to require any type of major surgery during the study.
31) Optional: Have a history of symptomatic herpes zoster within 16 weeks of randomization or any history of disseminated herpes simplex, disseminated herpes zoster, ophthalmic zoster, or varicella zoster virus central nervous system infections.
32) Optional: Received a live-attenuated vaccine within 4 weeks of randomization.
33) Optional: Are participants assigned female at birth who may wish to become pregnant and/or plan to undergo egg donation or egg harvesting for the purpose of current or future fertilization during the study and up to 14 days (or by the institutional guideline as indicated, whichever is longer) after the last dose of study drug administered during this clinical study.
34) Optional: Are participants assigned male at birth who have a female partner of childbearing age and unwilling to refrain from sperm donation for at least 14 days (or by the institutional guideline as indicated, whichever is longer) after last dose of study drug administered during this clinical study.
35) Optional: Requirement for ongoing therapy with or use of any prohibited medication as specified in the protocol (See Tables 19 and 20).

### Treatment Arms

Screening Period: Days −30 to −1
   Part 1A (Day 1 through Week 12):
Blinded Treatment: Day 1 through Week 12 (Part 1A)
   At baseline/randomization (Day 1), approximately 228 participants who meet protocol eligibility criteria will be randomized in a 1:1:1:1 ratio to 1 of 4 treatment groups as follows:
Treatment Group 1 (n=57): Formula (III) 200 mg orally once daily, on empty stomach
Treatment Group 2 (n=57): Formula (III) 75 mg orally once daily, on empty stomach
Treatment Group 3 (n=57): Formula (III) 25 mg orally once daily, on empty stomach
Treatment Group 4 (n=57): Placebo orally once daily, on empty stomach

### Week 12 Primary Analysis

At completion of Week 12 for all participants in Part 1A, a primary analysis of efficacy and safety will be performed.

Part 1B Treat-Through Groups (Immediately after Week 12 Visit Through Week 52):

Participants in the placebo group who complete the 12-week Part 1A treatment period will be eligible to be rerandomized in a double-blind manner at Week 12 to 1 of the 3 active treatment groups.

Rerandomization group: participants in the placebo group who complete the 12-week Part 1A treatment period will be eligible to undergo rerandomization in the double-blinded manner after the endoscopy assessment at Week 12 to 1 of the 3 active treatment groups.

### Part 1C (Weeks 52 Through Week 76):

Participants who complete Part 1B of the study will continue treatment extension for an additional 24 weeks. Participants will remain blinded during this period and receive the same dose of Formula (III) treatment as was assigned in Part B.

Consideration should be given to discontinuing the study drug for participants who show significant lack of clinical improvement per the investigator's judgment after Week 12. Consideration should also be given to discontinuing study drug for participants who meet disease worsening criteria between Week 12 and Week 76.

All participants may receive up to 76 weeks of Formula (III) study drug (12 weeks during Part A, 40 weeks during Part B, and 24 weeks during Part C). There will be a 4-week (28±2 days) follow-up period after the last dose of study drug to perform posttreatment assessments.

### Duration of Intervention:

Randomized participants in the study may receive a maximum of 76 weeks of treatment with Formula (III): 12 weeks during Part 1A, 40 weeks during Part 1B, and 24 weeks during Part 1C. This includes participants initially randomized to placebo, and rerandomized to active treatment at Week 12 and subsequent treatment through Week 76.

### Criteria for Study Drug Interruption:

When feasible, the medical monitor should be consulted prior to study drug interruption. The medical monitor must be informed as soon as possible of all cases of study drug interruptions for medical reasons. Prior to resumption of study drug, the investigator should discuss the case with the medical monitor.

Study drug interruption must occur in the following circumstances:
Intercurrent illness that would, in the judgement of the investigator, affect assessments of clinical status to a significant degree.
Positive urine pregnancy test.
Participant is scheduled for elective or emergency surgery (excluding minor skin procedures under local or no anesthesia), timing of study drug pausing should be determined in consultation with the medical monitor.
Any Grade 3 or above AE assessed as related to the study drug. The severity of AEs will be graded using the Common Terminology Criteria for Adverse Events (CTCAE) Toxicity Grading Scale, Version 5.0. For each episode, the highest grade attained should be reported as defined in the Toxicity Grading Scale.
Positive subjective PML and objective PML checklists, administered at a scheduled or unscheduled visit, prompts neurologist evaluation for PML. If PML is ruled out, then study drug should be restarted as described in the RAMP program.
During the time of study drug interruption for any of the above, the participant may continue to have study visits and to take part in procedures and assessments if deemed medically appropriate by the investigator.

### Dosage and Administration

In Part 1 of the study, study drug will consist of Formula (III) 100-mg, 75-mg, and 25-mg tablets, and Formula (III) PTM tablets for oral administration on an empty stomach (see dietary restrictions below).

### Part 1A Treatment Period

The tablets will be administered to each treatment group in the Part 1A treatment period, Day 1 through Week 12, as follows:
Treatment Group 1 (Formula (III) 200 mg): 1 Formula (III) 100-mg tablet, 1 Formula (III) Formula (III) 75-mg tablet, and 1 Formula (III) Formula (III) 25-mg tablet, orally once daily
Treatment Group 2 (Formula (III) 75 mg): 1 Formula (III) 75-mg tablet, 1 placebo-to-match (PTM) 100-mg tablet, and 1 PTM 25-mg tablet, orally once daily
Treatment Group 3 (Formula (III) Formula (III) 25 mg): 1 Formula (III) 25-mg tablet, 1 PTM 100-mg tablet, and 1 PTM 75-mg tablet, orally once daily
Treatment Group 4 (Placebo): 1 PTM 100-mg tablet, 1 PTM 75-mg tablet, and 1 PTM 25 mg tablet, orally once daily
In all cases, participants will take 3 tablets in total per day.

### Part 1B Treatment Period

The tablets administered to each treatment group in the Part 1B treatment period, Week 12 through Week 52, are as follows:
Treatment Group 1 (Formula (III) 200 mg): 1 Formula (III) 100-mg tablet, 1 Formula (III) 75-mg tablet, and 1 Formula (III) 25-mg tablet, orally once daily
Treatment Group 2 (Formula (III) 75 mg): 1 Formula (III) 75-mg tablet, 1 PTM 100-mg tablet, and 1 PTM 25-mg tablet, orally once daily
Treatment Group 3 (Formula (III) 25 mg): 1 Formula (III) 25-mg tablet, 1 PTM 100-mg tablet, and 1 PTM 75-mg tablet, orally once daily

### Part 1C Treatment Period

Participants entering Part C, Week 52 through Week 76, will remain in the same treatment group as was assigned in Part B.
In all cases, participants will take 3 tablets in total per day.

### Dietary Restrictions

Formula (III) should be administered without food on an empty stomach only with water, and no other medications or supplements should be taken with study drug; food can be consumed 4 hours before and 2 hours after the administration of Formula (III). Participants will be required to refrain from the consumption of iron and calcium at least 3 hours after the study drug. Participants should be instructed to administer study drug at or around the same time each day on an empty stomach. If one or more study drug tablets are not taken at the same time on a given day, participants should be instructed to take the missed tablets for that day as soon as possible during the same day. If one or more study drug tablets are not taken during the same day, participants should be instructed to not take any missed tablets on subsequent days so as to never exceed the regular dosage. Participants should note missed tablets in the drug diary. Participant's unused study drug should be returned to the site to account for study drug compliance.

### Statistical Methods:

### Primary Analysis (Week 12)

In part 1 a primary analysis will be conducted after all randomized participants have completed 12 weeks of blinded treatment or discontinued from the study and associated efficacy and safety assessments have been completed. These analyses will include all efficacy data collected at Week 12, and all of the accumulated safety data collected by the time the last randomized participant completes the Week 12 visit or discontinues from the study. If participants meet treatment failure criteria (i.e., need for rescue medications or surgical intervention for treatment of UC) or prematurely discontinue from the study treatment without available assessment result, or participants do not have sufficient measurements to determine efficacy endpoint, participants will be considered as not achieving clinical response. A Mantel-Haenszel test of the null hypothesis that the common risk difference is zero, stratified by randomization stratification factors, will be used for hypothesis testing of the primary endpoint. Nominal P values unadjusted for multiplicity will be calculated. For the evaluation of clinical response at Week 12, mMCS subscores (i.e., stool frequency, rectal bleeding, and endoscopic findings [by central readers]) at screening will be used as the baseline value.

Week 12 efficacy and safety analyses, including the primary analysis, will be performed.

### Final Analysis

A final analysis will be performed at the end of study.

Descriptive statistics for secondary endpoints at Week 52 and Week 76 will be provided within each Formula (III) group. For each binary endpoint, number and proportion of participants achieving respective criteria at Week 52 and Week 76 will be provided. No statistical comparisons will be performed.

### Safety

Safety analyses will include summaries of extent of exposure, AEs, laboratory evaluations, and vital sign assessments.

### Sample Size

A sample size of 57 participants per group in all active Formula (III) treatment groups and the placebo group will have at least 87% power to detect an absolute difference of 30.4% in clinical response between an individual treatment group and placebo at Week 12.

### Objectives and Endpoints:

### Primary Objectives:

To assess the efficacy of Formula (III), compared with placebo control, in achieving clinical response at Week 12.

### Primary Endpoints:

Clinical response at Week 12. Clinical response is defined as a decrease from baseline in the mMCS of ≥2 points and at least a 30% reduction from baseline, and a decrease in rectal bleeding subscore of ≥1 from baseline or an absolute rectal bleeding subscore of 0 or 1.

### Secondary Objectives:

To evaluate the safety and tolerability of Formula (III), up to Week 76.
To assess the efficacy of Formula (III), compared with placebo control, in achieving clinical remission at Week 12.
To assess the efficacy of Formula (III), compared with placebo control, in achieving clinical remission at Week 52.
To assess the efficacy of Formula (III), compared with placebo control, in achieving histologic-endoscopic mucosal improvement at Week 12.
To assess the efficacy of Formula (III), compared with placebo control, in achieving mucosal healing at Week 12.
To assess the efficacy of Formula (III), compared with placebo control, in achieving endoscopic improvement at Week 12.
To assess the efficacy of Formula (III) in achieving partial mMCS remission at 76 week 76.

### Secondary Endpoints:

Incidence of TEAEs, SAEs, or deaths, and treatment-emergent laboratory abnormalities
Clinical remission at Week 12. Clinical remission is defined as an mMCS of ≤2, including a stool frequency subscore (SFS)≤1 and not greater than baseline, rectal bleeding subscore of 0, and centrally read endoscopic subscore≤1 (score of 1 modified to exclude friability).
Clinical remission at Week 52. Clinical remission is as defined above.
Histologic-endoscopic mucosal improvement at Week 12. Histologic-endoscopic mucosal improvement is defined as a Geboes score≤3.1 and endoscopic subscore of ≤1.
Mucosal healing at Week 12. Mucosal healing is defined as a Geboes score≤2B.1 and endoscopic subscore≤1.
Endoscopic improvement at Week 12. Endoscopic improvement is defined as an endoscopic subscore of ≤1.
Partial mMCS remission at Week 76. Partial mMCS remission is defined as a SFS≤1 and rectal bleeding subscore of 0.

### LIST OF ABBREVIATIONS

5-ASA 5-aminosalicylate
6-MP 6-mercaptopurine
A alemtuzumab
ADA Antidrug antibody
AE adverse event
ALT alkaline phosphatase
ARA acid reducing agent
AST Aspartate aminotransferase
AUC area under the concentration versus time curve
AUC_{inf} area under the concentration versus time curve extrapolated to infinite time, calculated as AUCₗₐₛₜ+(Cₗₐₛₜ/λ_{z})
AUCₗₐₛₜ area under the concentration versus time curve from time zero to the last quantifiable concentration
AUCₜₐᵤ area under the concentration versus time curve over the dosing interval
AUCₓ₋ₓₓ partial area under the concentration versus time curve from time “x” to time “xx”
AT advanced therapy
BCRP breast cancer resistance protein
BA bioavailability
Cₘₐₓ Cₘₐₓ maximum observed concentration of drug
Cₜₐᵤ observed drug concentration at the end of the dosing interval
C_{trough} concentration at the end of the dosing interval
C difficile Clostridium difficile
CD4 Clusters of differentiation 4
CD8 Clusters of differentiation 8
CD Crohn's disease
CI confidence interval
CKD chronic kidney disease
CKD-EPI Chronic Kidney Disease Epidemiology Collaboration
COVID-19 coronavirus disease 2019
CR complete response
CRF case report form
CSR clinical study report
CTCAE Common Terminology Criteria for Adverse Events
CTD connective tissue disease
CV coefficient of variation
% CV % coefficient of variation
CYP Cytochrome P450
DMC data monitoring committee
DDI Drug-drug interaction
DNA deoxyribonucleic acid
ECG electrocardiogram
eCRF electronic case report form
EDC electronic data capture
EFD Embryo-fetal development
ECG Electrocardiogram
eDiary Electronic Diary
eGFR Estimated glomerular filtration rate
eGFRcr Estimated glomerular filtration rate based on serum creatinine
EoT End of treatment
EQ-5D-5L EuroQol (5 dimensions, 5 levels)
EQ VAS EQ visual analogue scale
EQ-5D EuroQOL (5 dimensions)
ET early termination
EU European Union
FDA Food and Drug Administration
FFPE formalin fixed and paraffin embedded
FSH follicle-stimulating hormone
GCP Good Clinical Practice
GI gastrointestinal
Gilead Gilead Sciences/Gilead Sciences, Inc.
GS glutamine synthetase
HA hyaluronic acid
HBcAb hepatitis B core antibody
HbsAg hepatitis B surface antigen
HBV hepatitis B virus
HCV hepatitis C virus
HIV human immunodeficiency virus
His histidine
HRQOL Health-Related Quality of Life
IB investigator's brochure
IAC Independent Adjudication Committee
IBD inflammatory bowel disease
IBDQ Inflammatory Bowel Disease Questionnaire
ICF informed consent form
ICH International Council for Harmonization
IEC independent ethics committee
Ig immunoglobulin
IL interleukin
IN integrase
IND investigational new drug
INR International normalized ratio
IRB institutional review board
IRT interactive response technology
IV Intravenously
JCV John Cunningham virus
LC-MS/MS liquid chromatography-tandem mass spectrometry
LLOQ lower limit of quantitation
MAD multiple-ascending dose
MAdCAM-1 mucosal address in cell adhesion molecule-1
MCS Mayo Clinic Score
mMCS modified Mayo Clinic Score
NSF normal stool frequency
OATP organic anion transporting polypeptide
P passage
PD Pharmacodynamics
PGA Physician's Global Assessment
P-gp P-glycoprotein
PI Principle investigator
PK pharmacokinetic
PML progressive multifocal leukoencephalopathy
PR electrocardiogramterval occurring between the onset of the P wave and the QRS complex representing time for atrial and ventricular depolarization, respectively
PRES posterior reversible encephalopathy syndrome
PRO patient-reported outcome
PS Patient Safety
PTM placebo-to-match
PT prothrombin time
PTT partial thromboplastin time
PTx Post treatment
QRS Electrocardiogramaction between the beginning of the Q wave and termination of the S wave, representing time for ventricular depolarization and repolarization to occur
QT Electrocardiogramterval between the beginning of the Q wave and the termination of the T wave, representing the time for both ventricular depolarization and repolarization to occur
RAT randomized and treated
RAMP risk assessment and minimization plan
RO receptor occupancy
SAC Safety Assessment Committee
SAD single-ascending dose
SADR serious adverse drug reaction
SAE serious adverse event
SAP statistical analysis plan
SC Subcutaneously
SD Standard deviation
SFS Stool frequency subscore
spp Species (plural)
SSR special situation report
T time
t_{1/2} terminal elimination half-life
TB tuberculosis
TEAE treatment-emergent adverse event
TNFα Tumor necrosis factor-alpha
UC Ulcerative Colitis
UC-PRO-SS ulcerative colitis patient-reported outcome signs and symptoms
ULN upper limit of normal
US United States
WBC white blood cell

1. (canceled)

2. A method for treating and/or preventing inflammatory bowel disease (IBD) in a subject, comprising co-administering to the subject:
A. a therapeutically effective amount of an α4β7 integrin small molecule inhibitor, wherein the α4β7 integrin small molecule inhibitor is selected from a compound of Formula (I), Formula (II), Formula (III), or Formula (IV) or a pharmaceutically acceptable salt thereof; and
B. a therapeutically effective amount of an anti-IL-12/IL-23 antibody or an antigen-binding fragment thereof.

3. The method of claim 2, wherein the α4β7 integrin small molecule inhibitor is a compound of Formula (III): 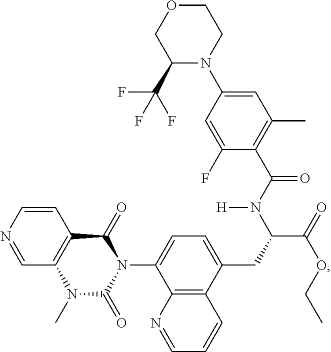
or a pharmaceutically acceptable salt thereof.

4. The method of claim 2, wherein the anti-IL-12/IL-23 antibody or antigen-binding fragment thereof comprises:
I. a heavy chain comprising (a) a heavy chain complementarity-determining region (HCDR) 1 comprising SEQ ID NO: 1; (b) a HCDR2 comprising SEQ ID NO: 2; and (c) a HCDR3 comprising SEQ ID NO: 3; and
II. a light chain comprising (a) a light chain complementarity-determining region (LCDR) 2 comprising SEQ ID NO: 4; (b) a LCDR2 comprising SEQ ID NO: 5; and (c) a LCDR3 comprising SEQ ID NO: 6.

5. The method of claim 2, wherein the anti-IL-12/IL-23 antibody or antigen-binding fragment thereof comprises:
I. a heavy chain comprising a heavy chain variable region (VH) sequence that is at least about 90%, 91%, 92%, 93%, 94%, 95%, 96%, 97%, 98%, 99% or 100% identical to the amino acid sequence of SEQ ID NO: 7; and
II. a light chain comprising a light chain variable region (VL) sequence that is at least about 90%, 91%, 92%, 93%, 94%, 95%, 96%, 97%, 98%, 99% or 100% identical to the amino acid sequence of SEQ ID NO: 8.

6. The method of claim 2, wherein the method comprises administering the α4β7 integrin small molecule inhibitor at a dose of between about 20 mg to about 500 mg.

7. The method of claim 2, wherein the method comprises administering the α4β7 integrin small molecule inhibitor at a dose of about 25 mg.

8. The method of claim 2, wherein the method comprises administering the α4β7 integrin small molecule inhibitor at a dose of about 75 mg.

9. The method of claim 2, wherein the method comprises administering the α4β7 integrin small molecule inhibitor at a dose of about 100 mg.

10. The method of claim 2, wherein the method comprises administering the α4β7 integrin small molecule inhibitor at a dose of about 200 mg.

11. The method of claim 2, wherein the α4β7 integrin small molecule inhibitor is administered orally.

12. The method of claim 2, wherein the α4β7 small molecule inhibitor is dosed once daily.

13. The method of claim 2, wherein the α4β7 integrin small molecule inhibitor is provided in a solid dosage form.

14. The method of claim 2, wherein the α4β7 integrin small molecule inhibitor is administered under fasting conditions.

15. The method of claim 2, wherein fasting conditions means the subject does not consume food within 4 hours before administration of the α4β7 integrin small molecule inhibitor and within 2 hours after administration of the α4β7 integrin small molecule inhibitor.

16. The method of claim 2, wherein the inflammatory bowel disease is ulcerative colitis (UC).

17. The method of claim 2, wherein the subject has received a prior treatment of one or more conventional or advanced therapies for UC.

18. The method of claim 2, wherein the inflammatory bowel disease is Crohn's disease.

19. The method of claim 2, wherein treating the IBD results in corticosteroid-free clinical remission of the IBD.

20. The method of any one of claims 1 to 5 claim 2, wherein treating the IBD results in clinical remission of the IBD.

21. The method of claim 2, wherein treating the IBD results in mucosal healing.

22. The method of claim 2, wherein the subject is a human.

23. The method of claim 2, wherein the subject does not suffer from chronic or active infections.

24. A method for determining a clinical response of a subject with moderate to severe UC to an α4β7 integrin small molecule inhibitor, comprising:
(a) determining one or more of a baseline mMCS a biological sample obtained from the subject and a baseline rectal bleeding subscore;
(b) administering to the subject a therapeutically effective amount of an α4β7 integrin small molecule inhibitor;
(c) determining one or more of a treatment mMSC score and a treatment rectal bleeding score from the subject; and
(d) generating a clinical response determination based upon one or more of a comparison of the baseline mMCS score to the treatment mMCS score and a comparison of the baseline rectal bleeding subscore to the treatment rectal bleeding subscore.

25. The method of claim 24, wherein the baseline mMCS score is 5 to 9 points.

26. The method of claim 24, wherein the baseline rectal bleeding subscore is ≥1.

27. The method of claim 24, wherein the clinical response determination is a positive clinical response characterized by a treatment mMCS score decrease of ≥2 points and a reduction of at least 30% from the baseline mMCS.

28. The method of claim 24, wherein the clinical response determination is a positive clinical response characterized by a treatment rectal bleeding subscore decrease of ≥1 compared to the baseline rectal bleeding score or an absolute rectal bleeding subscore of 0 or 1.

29. The method of claim 2, further comprising monitoring the subject after administration of one or more doses the α4β7 integrin small molecule inhibitor.

30. The method of claim 2, wherein monitoring the subject comprises obtaining one or more biological samples from the subject to determine if the patient has a clinical response.

31. The method of claim 2, wherein one or more biological samples are obtained from the subject at least 12 weeks after administration of the first dose of the α4β7 integrin small molecule inhibitor.

32. A method of determining clinical responsiveness of a subject with moderate to severe UC to an α4β7 integrin small molecule inhibitor and an anti-IL-12/IL-23 antibody combination therapy, comprising:
(a) determining one or more of a baseline mMCS a biological sample obtained from the subject and a baseline rectal bleeding subscore;
(b) administering to the subject a therapeutically effective amount of an α4β7 integrin small molecule inhibitor;
(c) determining one or more of a treatment mMSC score and a treatment rectal bleeding score from the subject; and
(d) generating a clinical response determination based upon one or more of a comparison of the baseline mMCS score to the treatment mMCS score and a comparison of the baseline rectal bleeding subscore to the treatment rectal bleeding subscore.

33. The method of claim 32, wherein the baseline mMCS score is 5 to 9 points.

34. The method of claim 32, wherein the baseline rectal bleeding subscore is ≥1.

35. The method of claim 32, wherein the clinical response determination is a positive clinical response characterized by an mMCS treatment score decrease of ≥2 points and a reduction of at least 30% from the baseline mMCS.

36. The method of claim 32, wherein the clinical response determination is a positive clinical response characterized by a treatment rectal bleeding subscore decrease of ≥1 compared to the baseline rectal bleeding score or an absolute rectal bleeding subscore of 0 or 1.
